(19) United States
(12) Patent Application Publication
(10) Pub. No.: US 2021/0017611 A1
(43) Pub. Date: Jan. 21, 2021

(54) METHOD AND KIT FOR HBV-HOST JUNCTION SEQUENCE IDENTIFICATION, AND USE THEREOF IN HEPATOCELLULAR CARCINOMA CHARACTERIZATION

(71) Applicant: JBS Science Inc., Doylestown, PA (US)

(72) Inventors: Selena LIN, Doylestown, PA (US); Wei SONG, Doylestown, PA (US)

(73) Assignee: JBS Science Inc., Doylestown, PA (US)

(21) Appl. No.: 16/932,434

(22) Filed: Jul. 17, 2020

Related U.S. Application Data
(60) Provisional application No. 62/875,059, filed on Jul. 17, 2019.

(51) Int. Cl. C12Q 1/70

(52) U.S. Cl. CPC C12Q 1/706

(57) ABSTRACT

A method and a kit for identifying HBV-host junction sequences (HBV-JSs) from a biological sample are provided. The method includes: preparing a DNA sample (e.g. DNA library) and performing at least one round of enrichment. Each round of enrichment includes a sub-step of capturing HBV DNA sequence-containing DNA molecules from the DNA sample by means of an HBV probe set, which includes a plurality of elaborately designed HBV primers configured to selectively and respectively target different regions of an HBV genome, and each HBV primer is labelled with an immobilization portion such as biotin moiety so as to allow immobilization onto a solid support such as magnetic beads. The method and kit can be used for non-invasively detecting HBV-JSs using a urine sample and other body fluids. The information of the HBV-JSs can be further utilized in the screening, diagnosis, prognosis and management of HBV-associated HCC.

### CROSS-REFERENCE TO RELATED APPLICATION

The present application claims priority to the U.S. provisional patent application No. 62/875,059, filed Jul. 17, 2019, whose content is hereby incorporated by reference in its entirety.

### TECHNICAL FIELD

This present disclosure relates generally to the field of biotechnology, specifically to genetic biomarkers that are associated with human cancers, and more specifically to methods and kits for identifying hepatitis B virus (HBV)-host junction sequences in tissue or body fluid samples and their use in screening, diagnosis, monitoring, management, and therapy of for hepatocellular carcinoma (HCC).

### BACKGROUND

Chronic hepatitis B virus (HBV) infection remains a global health burden despite the availability of a preventive vaccine, affecting more than 240 million people worldwide and associated with more than 600,000 deaths annually. HBV is the major etiology of hepatocellular carcinoma (HCC), associating with over 50% of HCC cases worldwide and up to 70-80% of cases in HBV-endemic areas such as sub-Sahara Africa and Asian countries. HCC is the fifth most common cancer worldwide and the most frequent cancer in certain parts of the world. HCC surveillance programs have been implemented to screen high-risk populations, including HBV-infected individuals, for the early detection of HCC. Regardless of these efforts, most cases of HCC remain undetected until late stages, resulting in poor prognosis. The current lack of a sensitive and convenient screening method provides an urgent need for improved early detection strategies of HCC.

During the course of infection, the HBV genome can integrate into the host chromosome. Integrated DNA was detected in more than 85% of HBV related HCC cases (HBV-HCC). Although it is known that viral breakpoints predominately occur in the DR1-2 region of the HBV genome, the integrated sites in the host DNA vary. Thus, each HBV integration event generates a unique HBV-host junction sequence (HBV-JS) that essentially creates a fingerprint of each infected hepatocyte. Thus, HBV-JSs can be used as a unique marker to trace for the HBV-HCC DNA that is released into the circulation and filtered into urine as fragmented LMW DNA.

Circulating cell-free DNA (cfDNA) has been identified in biological fluids. For example, in urine, two species are seen: a high-molecular-weight (HMW) DNA, greater than 1 kb, derived mostly from sloughed off cell debris from the urinary tract, and a low-molecular-weight (LMW) DNA, approximately 150 to 250 base pairs (bp), derived primarily from apoptotic cells.

Methods for analysis of HBV-JS fingerprints (integration sites) from genomic DNA have become readily accessible to researchers through the increasing availability of high-throughput next generation sequencing (NGS). As tools to identify viral integration sites have emerged, they are not entirely appropriate to the majority of scientific researchers, as they are not packaged in an intuitive interface, are time intensive, and are not entirely accurate. Thus, there remains a need to provide widespread accessibility to a method that enables users to accurately identify HBV-JSs in a time sensitive manner.

### SUMMARY

In a first aspect, the present disclosure provides a method for identifying at least one HBV-host junction sequence (HBV-JS) from a biological sample of a subject.

The method includes the following steps: (1) preparing a DNA sample from the biological sample; and (2) performing at least one round of enrichment over the DNA sample. Each round of enrichment in step (2) includes a sub-step of capturing HBV DNA sequence-containing DNA molecules from the DNA sample by means of an HBV probe set. The HBV probe set includes a plurality of HBV primers (also called HBV probes) having sequences thereof selectively and respectively corresponding to different regions of an HBV genome, and each HBV primer is labelled with an immobilization portion configured to allow immobilization onto a solid support.

Herein, the subject can be a primate such as a human, a monkey, a chimpanzee, a gorilla, etc. The biological sample can be a tissue sample such as a tissue biopsy sample or a liver cell line sample, and the biological sample can be a fluid sample, selected from a group consisting of a saliva sample, a nasopharyngeal sample, a blood sample, a serum sample, a plasma sample, gastrointestinal fluid, a bile sample, a cerebrospinal fluid sample, a pericardial sample, a vaginal fluid sample, a seminal fluid sample, a prostatic fluid sample, a peritoneal fluid sample, a pleural fluid sample, a synovial fluid sample, an interstitial fluid sample, an intracellular fluid sample, a cytoplasm sample, a lymph sample, a bronchial secretion sample, a mucus sample, a vitreous tumor sample, an aqueous humor sample, saliva sample, and a urine sample. Preferably the biological sample is a plasma sample, and more preferably, it is a urine sample, and under this latter circumstance, the method disclosed in this application allows for non-invasive detection of HBV-JSs so to provide important information regarding the screening, diagnosis, maintenance, prognosis, and management of HBV-associated HCC.

Herein, the plurality of HBV primers are configured to contain sequences therein that selectively and respectively corresponding to different regions of an HBV genome. To be more specific, each HBV primer can be designed to have a sequence that correspondingly matches with a particular HBV genomic region (e.g. having a sequence that may be at least 90% homologous with a sense strand or an anti-sense strand of the HBV genomic region) while having minimum homology with any host genomic region such that the each HBV primer can selectively hybridize with a sequence of a DNA molecule that corresponds to the HBV genomic region, thereby providing a means to selectively capture the HBV DNA sequence-containing DNA molecule. It is noted that the sequence homology between one HBV primer and its target HBV genomic sequence does not have to be 100% identical, as long as the hybridization therebetween is secure and strong enough to allow the specific capture of the target DNA molecule under an appropriate condition.

Herein, the HBV DNA sequence-containing DNA molecules can include DNA molecules that harbor a chimeric polynucleotide that includes both a host genomic DNA portion and an HBV genomic DNA portion (i.e. a host genome-integrated HBV genomic DNA), and can also include a polynucleotide whose sequence is purely HBV's.

In the method, the sub-step of capturing, by means of an HBV probe set, HBV DNA sequence-containing DNA molecules from the DNA sample can be through a primer extension capture (PEC) assay, which comprises:

denaturing the DNA sample to thereby obtain a denatured DNA sample by, e.g., heating at 95° C. for several minutes;

contacting the plurality of HBV primers with the denatured DNA sample for annealing by, e.g., incubating at an appropriate temperature;

performing a primer extension reaction by, e.g., polymerization;

immobilizing the DNA molecules captured by the plurality of HBV primers; and

eluting the DNA molecules.

According to some embodiments of the method, each round of enrichment can further include a sub-step of amplifying the DNA molecules, which can be realized by PCR-based approach using appropriate primers:

In any of the embodiments of the method described above, each of the plurality of HBV primers comprises a sequence selected from a group consisting of SEQ ID NOS: 49-175. In other words, the HBV probe set or HBV probe panel includes a set of HBV primers that represent part of a whole list of the SEQ ID NOS: 49-175. More preferably, the HBV probe set include all of the 127 sequences in SEQ ID NOS: 49-175 to thereby provide a comprehensive coverage to substantially cover the entire HBV genome. Furthermore, each of the plurality of HBV primers in the HBV probe set is configured to selectively target a different region of the HBV genome, such that this particular HBV primer can hybridize with a corresponding HBV DNA fragment integrated to the host genome while having minimum level of off-target effect to the host genome so as to provide a means for the specific capture and enrichment of the DNA molecules containing the HBV DNA sequence.

According to some embodiments of the method, the step (1) of preparing a DNA sample from the biological sample comprises: constructing a DNA library from the biological sample. Herein, the DNA library can optionally be a double-stranded DNA (dsDNA) library, yet according to some other more preferred embodiments, the DNA library is an ssDNA library, allowing the capture and enrichment of not only both ssDNA and dsDNA molecules, but also the short fragmented DNA molecules (e.g. <150 bp), which are commonly found in cell-free DNA samples obtained from a liquid biopsy sample such as a urine sample or a plasma sample.

Optionally for the method disclosed herein, a number of the at least one round of enrichment can be more than one. In other words, in the method described above, more than one round of enrichment (i.e. step (2)) can be performed so as to increase the enrichment efficiency.

In the method, in step (1) of preparing a DNA sample from the biological sample, each DNA molecule obtained thereby comprises a pair of adaptors flanking a DNA fragment from the subject. Accordingly, in the sub-step of capturing, by means of an HBV probe set, DNA sequences comprising the at least one HBV-JS through a primer extension capture (PEC) assay, the DNA sequences are captured in presence of adaptor blockers which are configured to hybridize with the pair of adaptors so as to minimize off-target capture.

In the method, the PEC assay relies on the immobilization portion labelled on each of the plurality of HBV primers for the capture and enrichment of target DNA molecules, such that the immobilization portion can form a stable binding with a coupling partner conjugated onto surface of the solid support.

Such binding can optionally be non-covalent. For example, the immobilization portion can comprise a biotin moiety, and correspondingly, the coupling partner conjugated onto surface of the solid support can comprise at least one of streptavidin, avidin, or an anti-biotin antibody. Other examples of the immobilization portion-coupling partner pair can include, but is not limited to, a carbohydrate-lectin pair, an antigen-antibody pair and a negative charged group-positive charged group static interacting pair.

According to some other embodiments of the method, the immobilization portion can be configured to be able to form a covalent connection (or crosslinking) with a coupling partner conjugated onto surface of the solid support. As such, the immobilization portion and the coupling partner can respectively be one and another of a cross-linking pair. Examples of the cross-linking pair include an NHS ester-primary amine pair, a sulfhydryl-reactive chemical group pair (e.g. cysteines, or other sulfhydryls such as maleimides, haloacetyls, and pyridyl disulfides), an oxidized sugarhydrazide pair, photoactivatable nitrophenyl azide's UV triggered addition reaction with double bonds leading to insertion into C—H and N—H sites or subsequent ring expansion to react with a nucleophile (e.g., primary amines), or carbodiimide activated carboxyl groups to amino groups (primary amines), etc. The solid support can comprise at least one of a magnetic bead, a filter, a resin bead, a nanosphere, a plastic surface, a microtiter plate, a glass surface, a slide, a membrane, a microfluidic channel, a chip, or a matrix. Preferably, the immobilization portion labelled on each HBV primer in the HBV probe set is a biotin moiety; and the solid support comprises streptavidin magnetic beads.

The method may further include, after the at least one enrichment in step (2), steps of: (3) sequencing the DNA sequences; and (4) identifying the at least one HBV-JS. Herein, step (4) of identifying the at least one HBV-JS can be done through ChimericSeq.

In a second aspect, the present disclosure further provides a kit for identifying at least one HBV-host junction sequence (HBV-JS) from a biological sample of a subject, which can be utilized in implementing the method as described above.

The kit includes an HBV probe set, which comprises a plurality of HBV primers having sequences thereof selectively and respectively corresponding to different regions of an HBV genome, and each HBV primer is labelled with an immobilization portion. The kit further includes a solid support, which is conjugated with a coupling partner on a surface thereof, wherein the coupling partner is configured to form a secure coupling to the immobilization portion of each HBV primer to thereby allow immobilization of HBV DNA sequence-containing DNA molecules to the solid support.

According to some embodiments of the kit, each of the plurality of HBV primers comprises a sequence selected from a group consisting of SEQ ID NOS: 49-175. More preferably, the HBV primers included in the HBV probe set include HBV primers that cover all of the 127 HBV sequences as set forth in SEQ ID NOS: 49-175.

According to some embodiments, the kit can further include a pair of adaptors, which are configured to be ligated to two ends of each DNA molecule in the biological sample to thereby obtain a DNA library from the biological sample. Further optionally, the kit can further include at least one adaptor blocker, which is configured to hybridize with sequences corresponding to the pair of adaptors in the each DNA molecule in the DNA library so as to minimize off-target capture.

Herein, the DNA library can be a double-stranded DNA library, but more preferably can be a single-stranded DNA library.

Optionally, the kit can further include at least one pair of amplifying primers, configured to amplify the HBV DNA sequence-containing DNA molecules.

In the kit, the immobilization portion can comprise a biotin moiety, and the coupling partner comprises at least one of streptavidin, avidin, or an anti-biotin antibody. Preferably, the solid support comprises streptavidin magnetic beads.

The kit can further include a software for identifying the at least one HBV-JS from data obtained from a sequencing assay, and the software is preferably ChimericSeq.

In a third aspect, the present disclosure further provides a method for de novo identification of HBV-JS. The method comprises:

constructing a DNA library from a biological sample collected from a subject;

applying the kit and the method according to the various embodiments as described above to enrich for HBV DNA sequence-containing DNA molecules;

sequencing the enriched DNA molecules and analyzing a sequencing result; and

if the sequencing result shows that a particular HBV-JS does not match with re-curated HBV-JS in a database, depositing the HBV-JS in the database.

In a fourth aspect, the present disclosure further provides a method for identification of an HBV-related HCC driver gene, or to be more specific, for determining if a candidate HBV-JS is a potential HCC driver. The method comprises:

applying the kit and method as described above to enrich and sequence HBV DNA sequence-containing DNA molecules from a DNA sample obtained from a population of subjects;

determining, if a sequencing result indicates that an HBV-JS is recurrent, that the HBV-JS is a candidate HBV-related HCC driver.

In any of the above methods, the biological sample can be a tissue sample or a liquid sample (e.g. urine sample), and the DNA library is preferably an ssDNA library.

In a fifth aspect, the present disclosure further provides a method for evaluate a risk of a subject for HBV-associated HCC. The method comprises:

collecting a biological sample from the subject;

constructing a DNA library from a biological sample;

applying the kit and method as described above to enrich and sequence HBV DNA sequence-containing DNA molecules in the DNA library;

identifying all HBV-JSs based on the sequencing result to thereby establish an HBV-JS profile for the subject; and

evaluating the risk of the subject for HCC based on the HBV-JS profile.

Herein, the biological sample can be any sample, but preferably a urine sample. The DNA library can be any type, but preferably an ssDNA library. The evaluating step can be based a multivariable analysis which includes, in addition to the HBV-JSs, other independent variables such as age, family history, pre-condition, etc.

### BRIEF DESCRIPTION OF THE DRAWINGS

FIG. 1 is an illustration of the detection of major HBV-JSs in urine of HBV-infected individuals as a marker for HBV-HCC screening and uncontrolled clonal expansion;

FIG. 2 illustrates the sensitivity of the 5′ biotinylated HBV primer extension enrichment using SEQ ID NO: 29, 31 and 33;

FIG. 3 illustrates the fold enrichment of the 5′ biotinylated HBV primer enrichment using SEQ ID NO: 29, 31 and 33;

FIGS. 4A and 4B together show a table presenting major HBV-JSs detected in HCC tissue by HBV DR1-2 enriched NGS analysis;

FIGS. 5A-5M illustrate the validation of major HBV-JSs identified from the NGS analysis;

FIG. 6 is a table presenting the characterization of validated HBV-JSs identified from NGS;

FIGS. 7A and 7B illustrate the detection of five unique HBV-JSs detected in matched HBV-HCC tissue and urine samples, respectively;

FIGS. 8A and 8B illustrate the detection of a rearranged HBV-JSs detected in matched HBV-HCC tissue and urine sample, respectively;

FIG. 9 illustrates the detection of HBV DNA in HBV-HCC tissue and urine samples;

FIG. 10 illustrates the detection of HBV-JS load in urine of HBV-Infected patients;

FIGS. 11A and 11B show the landscape of HBV DNA in urine of patients with or without HBV-JS, respectively;

FIG. 12 illustrates the reduced complexity of HBV-JSs in urine of HCC patients compared to non-HCC patients;

FIG. 13 illustrates the schematic overview of the ChimericSeq workflow;

FIG. 14 illustrates the description of the graphical user interface (GUI) for ChimericSeq;

FIG. 15 is a table describing the detection efficiency of HBV-JSs with defined lengths of HBV insert;

FIG. 16 is a table describing the evaluation of HBV-JSs from NGS data of HBV-infected patients;

FIG. 17 illustrates a schematic of primer extension capture (PEC) for HBV enrichment;

FIG. 18 shows mapping of the set of short primers with minimal overlap with human homologous regions containing high melting temperatures;

FIG. 19 compares the total NGS reads obtained by the ssDNA library vs dsDNA library construction;

FIG. 20 compares the HBV read % obtained by the ssDNA library vs dsDNA library construction;

FIG. 21 illustrates a flow chart for sequential PEC enrichment;

FIG. 22 illustrates a proposed application for detection of major HBV-JS in urine of HBV-HCC patients for HCC disease management;

FIGS. 23A-23C respectively show the primer extension capture (PEC) approach adopted to the HBV DNA libraries, the regions of sequence similarity between the human genome and the 3.2 Kb viral HBV genome, and the set of short primers with minimal overlap with human homologous regions containing high melting temperatures;

FIGS. 24A and 24B illustrate the Detection of HBV-JSs in matched tissue and urine among which, FIG. 18A shows the outline of a PCR based assay where a nested junction PCR approach was used to confirm the integration site for Patient 8, HBV and human primers were used to generate a first amplicon (1^{st} PCR) that is followed by a nested primer set to generate a second amplicon (2^{nd} PCR), and both urine cfDNA (U) and tissue DNA (T) samples were compared; and FIG. 18B shows the outline of a PCR based assay where a nested PCR followed by restriction endonuclease (RE) digestion approach was used to confirm integration sites, where patient samples were amplified with HBV and human primers, creating an amplicon with an identifiable RE cleavage site within the amplicon sequence, the amplicon was incubated in the absence (−) or presence (+) of the respective RE, and adapter-ligated tissue DNA library (NGS) and adapter-ligated HepG2 (HepG2) DNA served as positive and negative controls, respectively;

FIGS. 25A and 25B illustrate the identification of a rearranged HBV-JS in matched tissue and urine DNA among which, FIG. 25A shows the sequence of the HBV-JS with Chromosome 10 (Chr10) in patient 9, where amplification of this junction sequence using HBV and Chr10 primers resulted in a 23 bp difference between urine cfDNA (U) and tissue DNA (T) samples, and the Sanger sequence of inserted 23 bp sequence in urine DNA is depicted; and FIG. 25B shows the detection of the HBV-JS with Chromosome 5 (Chr5) in the corresponding tissue, where amplification of tissue DNA of this junction sequence using HBV and hybrid Chr5-Chr10 primers followed by Sanger sequencing confirmed the same inserted 23 bp sequence in tissue DNA, and HepG2 DNA was used as the negative control;

FIGS. 26A-26C illustrate the meta-analysis of HBV-JSs reveals recurrent targeted genes among which, FIG. 26A shows the frequency of HBV integrated host genes compiled from literature reports and our study, where fifty-one host genes were identified at or near HBV integration sites and are displayed along the x-axis in order of increasing frequencies (denoted by the numbers along the y-axis), genes reported in at least two separate studies (recurrent targeted genes) are denoted by an asterisk (*), and the number in parentheses indicates the contribution from our study; FIG. 26B shows the map of TERT integration sites along the human and HBV genomes, where 67 TERT integration sites, represented by a black dot, were plotted at the breakpoints of the TERT gene along the x-axis and breakpoints of HBV along the y-axis, this analysis was compiled from 56 patients diagnosed with HCC, of which 5 came from our study, TERT integration sites were mapped in to the HBV (NC_003977.1) and human (GRCh38.p2) reference genomes, the coordinates of the x-axis decreases from 1,315 kb to 1,275 kb to represent the direction of the transcriptional start site from a 5′-3′ orientation, and the bottom panel represents an expanded view of TERT integration sites along the human genome position 1,296 kb to 1,295 kb; and FIG. 26C shows the overview of TERT integration sites and TERT promoter mutations identified from the 23 HCC patients in our study, where gray boxes denote a positive status and white boxes denote a negative or undetectable status, * denotes patients with HBV integration in the TERT promoter, and patients with the TERT hotspot promoter mutation indicated by base position before ATG start;

FIG. 27 shows the proposed model for how reduced complexity of HBV integration sites indicates clonal expansion and HCC development;

FIGS. 28A-28C show the top five significantly enriched Gene Ontology terms associated with RTG genes based on EnrichR software: (FIG. 28A) Biological processes, (FIG. 28B) Molecular function, and (FIG. 28C) Drug Signatures Database (DSigDB), where pathways are presented based on combined EnrichR score, and DSigDB relates drugs/compounds to their target genes;

FIGS. 29A and 29B show the distribution of integration breakpoints in the HBV genome in (FIG. 29A) HCC tumor samples and (FIG. 29B) Adjacent tumor samples, where a total of 3,052 and 5,259 HBV breakpoints were available from tumor and adjacent tumor samples, respectively, and each histogram represents the frequency of integration breakpoints at different loci in the HBV genome (nt. 1-3215) as numbered in the outer ring;

FIGS. 30A-30C show the mapping of TERT, MLL4, and PLEKH4G4B HBV integration breakpoints along the human and HBV genomes: FIG. 30A shows TERT breakpoints, where 219 TERT integration breakpoints derived from 161 unique patients are plotted, the y-axis coordinates decrease from 1,320 kb to 1,260 kb to represent the direction of the transcriptional start site from a 5′-3′ orientation, and the expanded view of the region with the most integration sites is shown for the human genome position 1,297 kb to 1,294 kb and the HBV nt. 1500-2000; FIG. 30B shows MLL4 breakpoints, where 115 MLL4 integration breakpoints are plotted and derived from 64 unique patients, and blue squares denoting exon regions are representatively shown; FIG. 30C shows PLEKH4G4B breakpoints, where 47 of the 116 reported PLEKHG4B breakpoints plotted are derived from 8 unique HCC patients, colored dots correspond to each unique patient, each dot represents the mapped locations of the integration sites where the human gene breakpoints (GRCh37) are located on the y-axis, and HBV breakpoints are located on the x-axis, in accordance with the reported locations; and

FIGS. 31A and 31B illustrate the TERT gene alterations identified in HBV-HCC tissues, with FIG. 31A shown for the in-house cohort (n=22), and FIG. 31B for the compiled HBV-HCC cohort, where patients are derived from our in-house (n=22) and from literatures (n=129) [24,26], and the number of HCC patients is indicated in parenthesis.

### DETAILED DESCRIPTION OF THE INVENTION

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

Various embodiments will be described in detail through the displayed figures. Reference to these embodiments does not limit the scope of the claims attached hereto. Provided examples are not meant to limit the scope of methods and claims herein, but rather describe example uses of the embodiments of the claims.

The terms “a,” “an,” and “the” as used herein include plural referents, unless the context clearly indicates otherwise.

The term “genome” and “genomic” refer to any nucleic acid sequences (coding and non-coding) originating from any living or non-living organism or single-cell. These terms also apply to any naturally occurring variations that may arise through mutation or recombination through means of biological or artificial influence. An example is the human genome, which is composed of approximately 3×109 base pairs of DNA packaged into chromosomes, of which there are 22 pairs of autosomes and 1 allosome pair.

The term “nucleotide sequence” as used herein indicates a polymer of repeating nucleic acids (Adenine, Guanine, Thymine, and Cytosine, and Uracil) that is capable of base-pairing with complement sequences through Watson-Crick interactions. This polymer may be produced synthetically or originate from a biological source.

The term “nucleic acid” refers to a deoxyribonucleotide (DNA) or ribonucleotide (RNA) and complements thereof. The size of nucleotides is expressed in base pairs “bp”. Polynucleotides are single- or double-stranded polymers of nucleic acids and complements thereof.

The term “deoxyribonucleic acid” and “DNA” refer to a polymer of repeating deoxyribonucleic acids.

The term “ribonucleic acid” and “RNA” refer to a polymer of repeating ribonucleic acids.

The term “disease” or “disorder” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint, or affectation.

As used herein, “cancer” refers to any stage of abnormal growth or migration of cells or tissue, including precancerous and all stages of cancerous cells, including but not limited to adenomas, metaplasias, heteroplasias, dysplasias, neoplasias, hyperplasias, and anaplasias.

As used herein, “cancer progression” refers to any measure of cancer growth, development, and/or maturation including metastasis. “Cancer progression” includes increase in cell number, cell size, tumor size, and number of tumors, as well as morphological and other cellular and molecular changes and other characteristics. As an example, one measure of cancer progression is the use of staging characteristics. As an additional example, one measure of cancer progression is the use of detecting expression, whether at the protein or mRNA level, of certain genes

The term “diagnosing” means any method, determination, or indication that an abnormal or disease condition or phenotype is present. Diagnosing includes detecting the presence or absence of an abnormal or disease condition, and can be qualitative or quantitative.

The term “gene” is well known in the art, and herein includes non-coding region such as promoter or other regulatory sequences or proximal non-coding region.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression/production of an antibody or antigen-binding fragment can be within the cytoplasm of the cell, and/or into the extracellular milieu such as the growth medium of a cell culture.

The term “biomarker” is an agent used as an indicator of a biological state. It can be a characteristic that is objectively measured and evaluated as an indicator of normal biological processes, pathogenic processes, or pharmacologic responses to a therapeutic intervention. A biomarker can be a fragment of genomic DNA sequence that causes disease or is associated with susceptibility to disease, and may or may not comprise a gene.

The term “low molecular weight” or LMW nucleic acid refers a nucleic acid, such as DNA, of less than 1000 base pairs, usually less than 300 base pairs.

The term “nucleotide amplification reaction” refers to any suitable procedure that amplifies a specific region of polynucleotides (target) using primers.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “chimeric reads” herein refers to a nucleotide sequence obtained from next generation sequencing, whereby the length of the read contains genomic material from two separate biological entities or chromosomes joined covalently through integration. For example, viruses can integrate viral nucleotide sequences into the genomic nucleotide sequence of a human host.

Throughout the disclosure, the terms “probe set”, “probe panel”, or alike, are considered to be exchangeable, and the term “HBV primer” mentioned in the HBV probe set is also considered to be exchangeable to “HBV probe”.

Due to the imprecision of standard analytical methods, molecular weights and lengths of polymers are understood to be approximate values. When such a value is expressed as “about” X or “approximately” X, the stated value of X will be understood to be accurate to ±10%.

Provided herein include methods and kits that can provide a sensitive, specific, and noninvasive platform for detecting HBV-JS in circulating nucleic acid sequences from a biological sample including body fluid or HBV-infected liver tissue DNA. Any HBV-JS DNA found in cell-free DNA isolated from a patient's body fluid can be used because it is representative of liver-derived DNA. The methods use a biotinylated HBV primer extension to enrich for HBV sequences of library DNA. The enriched libraries were analyzed for HBV-JS by NGS. As shown in the following examples, the methods are useful for HCC screening and monitoring of HBV-infected individuals. This method is particularly useful for high-risk HCC individuals and individuals with occult HBV infection to undergo frequent noninvasive screening to monitor disease progression, as they are often asymptomatic.

The present disclosure features at least the following three components used in developing an integrative HBV-JS analysis platform. First, a biotinylated HBV primer extension enrichment was used to enrich DNA samples for HBV DNA sequences that may contain HBV-JSs. Second, the enriched libraries are amplified by primers targeting all DNA template and sequenced by Illumina next generation sequencing platform. Lastly, the NGS data can be analyzed by ChimericSeq for identifying HBV-JSs, where the analysis results were successfully confirmed for an 87% validation rate (13/15).

Throughout the disclosure, the term “biological sample” can be deemed to comprise a tissue sample, such as a biopsy sample or a tissue culture sample. A biological sample may as well comprises biological fluids (i.e. liquid sample) including, but not limited to, saliva, nasopharyngeal, blood, plasma, serum, gastrointestinal fluid, bile, cerebrospinal fluid, pericardial, vaginal fluid, seminal fluid, prostatic fluid, peritoneal fluid, pleural fluid, urine, synovial fluid, interstitial fluid, intracellular fluid or cytoplasm and lymph, bronchial secretions, mucus, or vitreous or aqueous humor. Biological samples can also include cultured medium. In certain embodiments, the preferred biological fluid is urine, and in such cases, the method disclosed in this present application can be used to non-invasively detect HBV-JSs for HCC screening, cancer progression, and for HBV-HCC disease monitoring.

In certain embodiments, the platform uses biological samples containing fragmented circulation derived DNA known as “low molecular weight” (LMW) DNA. The DNA is low molecule weight because it is generally less than 300 base pairs in size. This LMW DNA is released into circulation through necrosis or apoptosis by both normal and cancer cells. It has been shown that LWM DNA is excreted into the urine and can be used to detect tumor-derived DNA, provided a suitable assay, such as a short template assay for which detection is available (Su Y H et al. 2008).

The inventions disclosed herein have the advantage that the procedures provided are capable of screening for HBV-related hepatocellular carcinoma, where unique major HBV-JSs serve as a marker of uncontrolled clonal expansion.

The methods described herein can be used to determine the status of an existing disease identified in a subject. For example, 19 HCC, 21 hepatitis and 19 cirrhosis urine samples were evaluated for HBV-JSs, and all HCC urine samples with HBV-JSs contained only integrated HBV sequences in the DR1-2 region, a higher load of HBV-JS, and a reduced HBV-JS complexity compared to non-HCC patients. Thus, the HBV-JS load and HBV-JS species detected in urine can be used to screen for HBV-HCC and monitor HBV related disease.

The methods described herein can be used to identify subject patients for treatment and to determine risk factors associated with HBV-JSs. Such methods can include, for example, determining whether an individual has relatives who have been diagnosed with a particular disease. Screening methods can also include, for example, conventional work-ups to determine familial status for a particular disease known to have a heritable component. Screening may be implemented as indicated by known patient symptomology, age factors, related risk factors, etc. These methods allow the clinician to routinely select patients in need of the methods described herein for treatment. In accordance with these methods, screening may be implemented as an independent program or as a follow-up, adjunct, or to coordinate with other treatments. Thus, the methods of the present inventions can be used for cancer screening, particularly for early detection, monitoring of recurrence, disease management, and to develop a personalized medicine regime for a cancer patient.

It is to be understood that the above described embodiments are merely illustrative of numerous and varied other embodiments which may constitute applications of the principles of the inventions disclosed herein. Other embodiments may be readily devised by those skilled in the art without departing from the spirit or scope of this invention and they shall be deemed within the scope of the disclosure.

The inventions provided in the disclosure is further illustrated by the following non-limiting examples.

### Example 1: Development of a Method for Detecting HBV-JSs and the Use of Major HBV-JSs in Urine as a Marker for HBV-HCC Screening and Uncontrolled Clonal Expansion

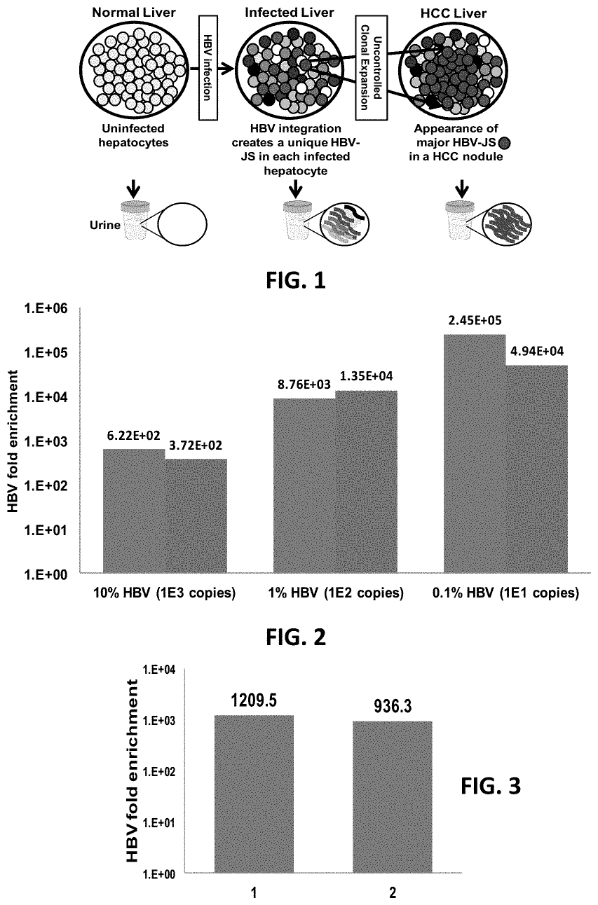

FIG. 1 is a schematic presentation of the detection of major HBV-JS in urine of HBV-infected individuals that can be utilized as a marker for HBV-HCC screening and uncontrolled clonal expansion.

In order to be able to reliably detect major HBV-JSs in urine samples, a biotinylated HBV primer extension enriched NGS assay was first developed. The following protocol was used: approximately 50-200 ng of tissue DNA was fragmented by sonication and subjected to NGS library DNA preparation as described by Ding et al. 2012 with minor modifications including 10 cycles of library DNA amplification (SEQ ID NO: 1, 2, 3) using Herculase II Fusion polymerase (Agilent Technologies, Santa Clara, Calif.). All the oligo sequences and reaction conditions for library preparation are listed in Table 1. To enrich for DNA that contains HBV DR1-2 DNA sequences, a multiplex biotin HBV primer extension reaction was performed using amplified library DNA in a reaction containing 1× Herculase II Buffer, 250 μM dNTP, and 20 pmol of biotinylated HBV primers as listed in Table 2. The reaction was held at the condition of 95° C. 2 mins, then 55° C. for 5 hrs with rotation. After a 5 hr incubation, 0.2 μl of heat inactivated Herculase II Fusion polymerase was added to each reaction and incubated at 55° C. for another 30 mins, followed by 72° C. for 90 s. The primer extended DNA was collected by using hydrophilic streptavidin magnetic beads (New England Biolabs, Ipswich, Mass.) as described by Gnirke et al. 2009 and used as the template in an indexing PCR (SEQ ID NO: 4 and 5) to add a unique barcode to each patient sample. Each indexed library was quantified and pooled accordingly for one NGS. NGS was performed to generate 150 bp paired-end reads on the Illumina MiSeq platform (Penn State Hershey Genomics Sciences Facility at Penn State College of Medicine, Hershey, Pa.). Sequences were analyzed using the ChimericSeq software to identify HBV-JSs.

TABLE 1
Oligos and reaction conditions for the preparation of HBV DR1-2 enriched library DNA.

| Primer | Primer | Tm | | |
|---|---|---|---|---|
| Name | Length | (° C.) | Sequence 5′-3′ | PCR conditions |
| Mod P_4F | 22 | 65 | CAAGCAGAAGACGGCA | 95° C. 2 mins, then 95° C. |
| | | | TAC*G*A (SEQ ID NO: 1) | 20s, 65° C. 20s, 58° C. 30s, |
| Mod P_3R | 20 | 66 | AATGATACGGCGACCA | 72° C. 20s for 10 cycles, |
| | | | CC*G*A (SEQ ID NO: 2) | and 72° C. 3 mins |
| Ad-Ad | 10 | 78 | +g-A+T+C+T+g+A+T+C+ | |
| LNA clamp | | | g-PH (SEQ ID NO: 3) | |

| Primer | | Primer | Tm | | |
|---|---|---|---|---|---|
| Name | Barcode | Length | (° C.) | Sequence 5′-3′ | PCR conditions |
| Indexed | TATAGCCT | 71 | 79 | AATGATACGGCGACCA | 95° C. 2 mins, then 95° C. |
| | ATAGAGG | | 80 | CCGAGATCTACACTANN | 20s, 60° C. 60s, 72° C. 20s |
| | C | | | NNNNNNacactctaccctacacg | for 8 cycles, and 72° C. 3 |
| | CCTATCCT | | 80 | acgctcttccgatc (SEQ ID NO: | mins. |
| | GGCTCTGA | | 81 | 4) | |
| | AGGCGAA | | 81 | | |
| | G | | | | |
| | GTACTGAC | | 80 | | |
| Mod | — | 24 | 67 | CAAGCAGAAGACGGCA | |
| P_R | | | | TACGAG*A*T (SEQ ID | |
| | | | | NO: 5) | |

′+′ denotes a modified locked nucleic acid nucleotide. -PH denotes a 3′ phosphorylation of the oligo. ′*′ denotes a phosphorothioate bond to prevent excision from the 3′-5′ exonuclease activity. Lower case sequences denote a 32 bp overlapping sequence between the HBV DR1-2 enrichment and indexing primers. ′R′ base denotes degenerate nucleotides containing A and G nucleotides. ′N′ denotes the designated sequence of the i5 barcode.

TABLE 2
5′ Biotinylated HBV primers in an HBV probe panel.

| Primer | Primer | | Tm | |
|---|---|---|---|---|
| Name | Region | Length | (° C.) | Sequence 5′-3′* |
| 1 | 3-34 | 30 | 68-71 | ACAACATTCCACCAARCTCTKCTAGATCCC (SEQ ID NO: 6) |
| 2 | 95-126 | 39 | 70 | AAGATTGACGATATGGWTGAGGCAGTAGTCGGAACAGGG |
| | | | | (SEQ ID NO: 7) |
| 3 | 201-240 | 40 | 73 | GGTATTGTGAGGATTTTTGTCAACAAGAAAAACCCCGCCT |
| | | | | (SEQ ID NO: 8) |
| 4 | 270-299 | 29 | 70-72 | GACACACGGGTGYTCCCCCTAGAAAATTG (SEQ ID NO: 9) |
| 5 | 382-356 | 30 | 69-74 | ACACATCCAGCGATARCCAGGACAAYTRGG (SEQ ID |
| | | | | NO: 10) |
| 6 | 456-486 | 30 | 69-70 | AGGTATGTTGCCCGTTTGTCCTCTAMTTCC (SEQ ID NO: 11) |
| 7 | 570-597 | 27 | 67-69 | TACAAAACCTWCGGACGGAAAYTGCAC (SEQ ID NO: 12) |
| 8 | 605-635 | 30 | 72-74 | CCCATCCCATCATCYTGGGCTTTCGCAARA (SEQ ID NO: 13) |
| 9 | 693-725 | 31 | 74 | AAACAGTGGGGGAAAGCCCTACGAACCACTG (SEQ ID |
| | | | | NO: 14) |
| 10 | 749-780 | 31 | 72 | GGTACTGGGGGCCAAGTCTGTACAACATCTT (SEQ ID |
| | | | | NO: 15) |
| 11 | 781-810 | 40 | 71 | GAGTCCCTTTATRCCGCTRTTACCAATTTTCTTTTGTCTT |
| | | | | (SEQ ID NO: 16) |
| 12 | 871-900 | 35 | 69 | CCCTTAACTTCATGGGATATGTAATTGGRAGTTGG (SEQ ID |
| | | | | NO: 17) |
| 13 | 951-980 | 40 | 69-72 | TTCCAATCAATAGGYCTGTTTACAGGCAGTTTCCKAAAAC |
| | | | | (SEQ ID NO: 18) |
| 14 | 1033-1077 | 45 | 69 | CAATGTGGMTATCCTGCTTTRATGCCTTTATATGCATGTAT |
| | | | | ACAA (SEQ ID NO: 19) |
| 15 | 1101-1130 | 30 | 69 | TGTTTACACAGAAAGGCCTTGTAAGTTGGC (SEQ ID NO: 20) |
| 16 | 1183-1212 | 30 | 80 | GCCCCAACCCGTGGGGGTTGCGTCAGCAAA (SEQ ID |
| | | | | NO: 21) |
| 17 | 1261-1290 | 30 | 73-75 | AGCKGCTAGGAGTTCCGCAGTATGGATCGG (SEQ ID |
| | | | | NO: 22) |
| 18 | 1342-1371 | 30 | 71 | GTTGTCCTCTCTCGGAAATACACCGCCTTT (SEQ ID NO: 23) |
| 19 | 1395-1424 | 30 | 76 | CAACTGGATCCTGCGCGGGACGTCCTTTGT (SEQ ID NO: 24) |
| 20 | 1513-1542 | 30 | 79 | CCGACCACGGGGCGCACCTCTCTTTACGCG (SEQ ID NO: 25) |
| 21 | 1575-1604 | 30 | 76 | ACGTGCAGAGGTGAAGCGAAGTGCACACGG (SEQ ID |
| | | | | NO: 26) |
| 22 | 1613-1629 | 17 | 67 | GACCACCGTGAACGCCC (SEQ ID NO: 27) |
| 23 | 1633-1653 | 21 | 64 | AGGTCTTGCCCAAGGTCTTAC (SEQ ID NO: 28) |
| 24 | 1650-1671 | 22 | 65 | TTGCACAACAGGACTCTTGGAC (SEQ ID NO: 29) |
| 25 | 1685-1719 | 25 | 68 | AACGACCGACCTTGAGGCATACTTC (SEQ ID NO: 30) |
| 26 | 1691-1720 | 31 | 69 | CCGACCTTGAGGCATACTTCAAAGACTGTTT (SEQ ID |
| | | | | NO: 31) |
| 27 | 1737-1754 | 17 | 56-60 | GAGTTRGGGGAGGAGAT (SEQ ID NO: 32) |
| 28 | 1741-167 | 26 | 64-67 | TRGGGGAGGAGATAAGGTTAAAGGTC (SEQ ID NO: 33) |
| 29 | 1828-1862 | 35 | 70 | CCTCTGCCTAATCATCTCATGTTCATGTCCTACTG (SEQ ID |
| | | | | NO: 34) |
| 30 | 1896-1930 | 35 | 71-72 | GGGGCATGGACATTGACCCSTATAAAGAATTTGGA (SEQ |
| | | | | ID NO: 35) |
| 31 | 1997-2026 | 30 | 75 | ACCGCCTCTGCTCTGTATCGGGAGGCCTTA (SEQ ID NO: 36) |
| 32 | 2081-2110 | 30 | 71-73 | TGTTGGGGTGAGTTGATGAATCTRGCCACC (SEQ ID NO: 37) |
| 33 | 2146-2190 | 45 | 70 | ATTTTTAGGCCCATATTAACRTTGACATAGCTGACTACTAA |
| | | | | TTCC (SEQ ID NO: 38) |
| 34 | 2221-2260 | 40 | 67-70 | CACCAAATAYTCAAGRACAGTTTCTCTTCCAAAAGTAAGR |
| | | | | (SEQ ID NO: 39) |
| 35 | 2305-2340 | 36 | 70 | GTAGTTTCCGGAAGTGTTGATAAGATAGGGGCATTT (SEQ |
| | | | | ID NO: 40) |
| 36 | 2380-2409 | 30 | 75 | TCCCTCGCCTCGCAGACGAAGGTCTCAATC (SEQ ID NO: 41) |
| 37 | 2466-2502 | 37 | 69 | TAGAAGAATAAAGCCCAGTAAAGTTTCCCACCTTATG |
| | | | | (SEQ ID NO: 42) |
| 38 | 2541-2580 | 40 | 66-69 | TTTCCTSACATTCATCTACAGGAGGACATTRTTRATAGAT |
| | | | | (SEQ ID NO: 43) |
| 39 | 2697-2740 | 44 | 70-72 | CCGTATTATCCWGARCATGCAGTTAATCATTACTTCAAAA |
| | | | | CTAG (SEQ ID NO: 44) |
| 40 | 2783-2812 | 30 | 68-72 | CAAAATGAGGCGCTRCGTGTAGTYTCTCTY (SEQ ID NO: 45) |
| 41 | 2851-2880 | 30 | 73 | GGAGGTTGGTCTTCCAAACCTCGACAAGGC (SEQ ID NO: 46) |
| 42 | 2931-2960 | 30 | 72-76 | CCAGTTGGACCCTGCRTTCRRAGCCAACTC (SEQ ID NO: 47) |
| 43 | 3181-3215 | 24 | 70 | TCATCCTCAGGCCATGCAGTGGAA (SEQ ID NO: 48) |

*All primers are labelled with a 5′ biotin modification. ′R′ base denotes redundant A + G base. ′Y′ base denotes redundant C + T base. ′W′ base denotes redundant A + T base. ′S′ base denotes C + G base.

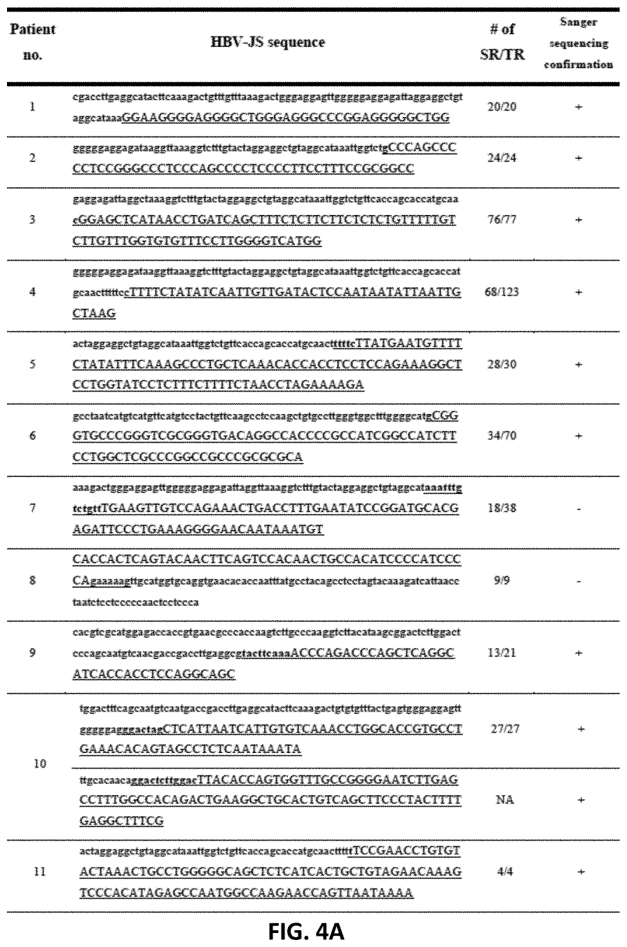

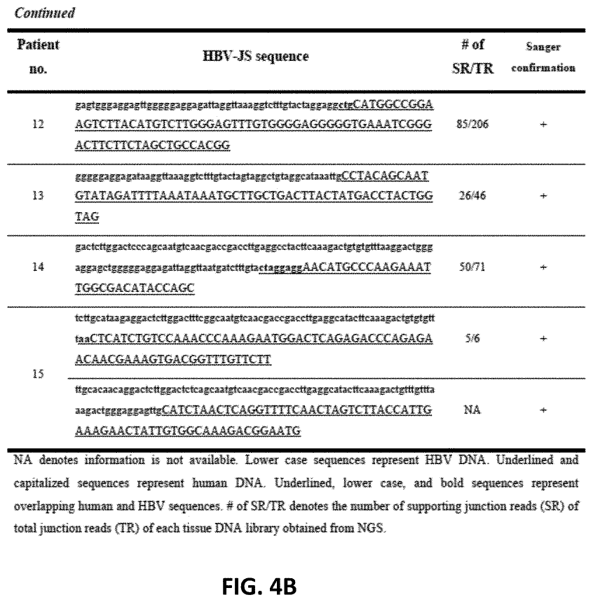

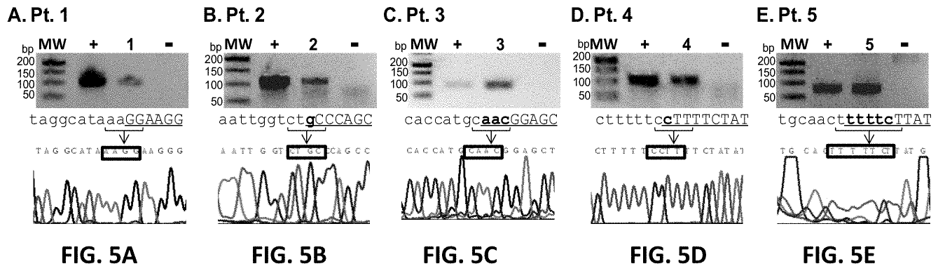

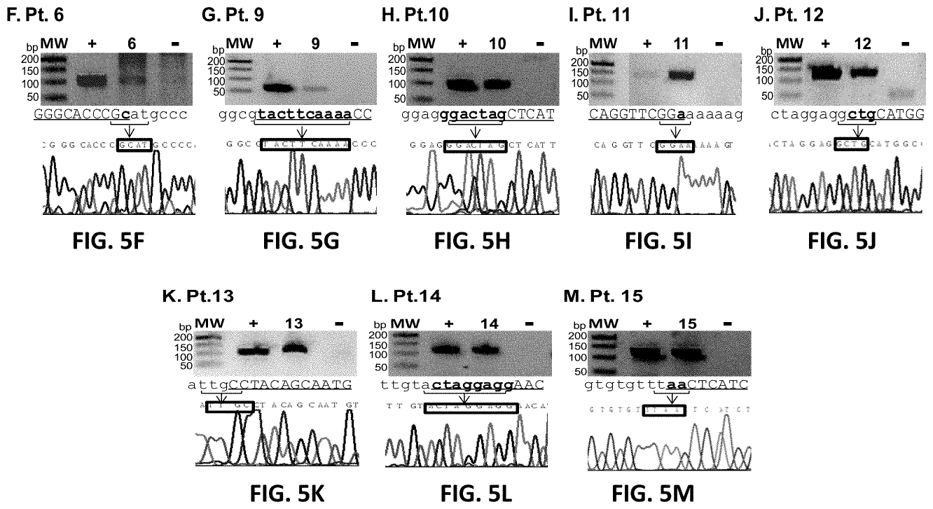

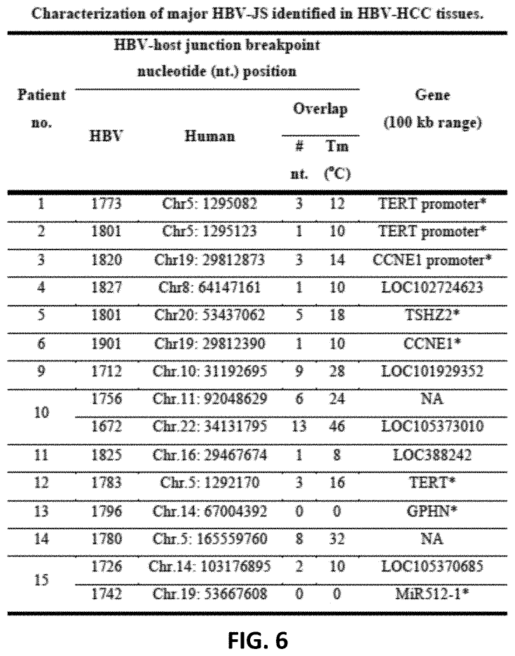

FIG. 2 shows HBV DNA sensitivity and fold enrichment of a multiplex biotinylated HBV primer extension approach. The ratio of library HBV nt. 1583-1791 DNA to library chromosome 1, a 71 bp sequence, DNA were used to calculate HBV DNA fold enrichment before and after a multiplex biotinylated HBV primer extension using three HBV biotinylated primers (SEQ ID NO: 29, 31, and 33). 10% HBV (1E3 copies), 1% HBV (1E2 copies), and 0.1% HBV (1E1 copies) denote the ratio of HBV library DNA in a background of chromosome 1 library DNA with the total input amount of HBV DNA denoted in parentheses (input copies). FIG. 3 shows HBV fold enrichment by biotinylated HBV primer extension. Duplicates (1,2) containing a mixture of HBV nt. 1583-1791 (˜1E5 copies) and chromosome 1 (˜5E4 copies) library DNA, a marker for non-specific enrichment, were enriched by biotinylated HBV (Biotin) primers (SEQ ID NO: 29, 31, and 33) in a primer extension reaction. The fold enrichment was calculated using the ratio of HBV/Chr1 before and after HBV enrichment. FIGS. 4A and 4B shows a table listing the major HBV-JSs, derived from HBV-HCC tissue, identified by ChimericSeq from a biotinylated HBV primer extension enriched NGS using SEQ ID: 29, 31, 33. FIGS. 5A-5M show Sanger sequencing validation of NGS identified HBV-JSs from HBV-HCC tissue DNA. Panels A to M depict the validation of NGS-identified HBV-JSs by the PCR-Sanger sequencing approach from patients 1-15, respectively. Tissue DNA from patients was subjected to PCR amplification using unique primers of the major junction sequences identified from NGS analysis (upper panel). An HBV-enriched tissue library DNA was used as the positive control (+) and DNA from HepG2 cells was used as the negative control (−). The amplicon from each sample was Sanger sequenced, and the depicted chromatogram contains the junction sequence selected with a black box (lower panel). Human and HBV DNA sequences are annotated as well. FIG. 6 shows a table summarizing the characterization of the 13 confirmed major HBV-JS derived from HBV-HCC tissue. The nucleotide positions of the HBV (NC_003977.1) and human (GRCh38.p2) genome sequences at the HBV-human junction breakpoints, along with the number of overlapping nt. identified, and the Tm (° C.) of the overlapped sequences determined by the JBS ChimericSeq software are listed. The closest genes identified within 100 kb of the junction breakpoint are listed, as defined by NCBI's RefSeq gene database. Junction sequences where no known gene was present within 100 kb are listed as “NA”. ‘*’ denotes genes known to associated with carcinogenesis (Horikawa I et al. 2001; Donnellan R et al. 1999; Ozawa T et al. 2004; Yamamoto M et al. 2011; Wang W et al. 2012; and Harel S A et al. 2015).

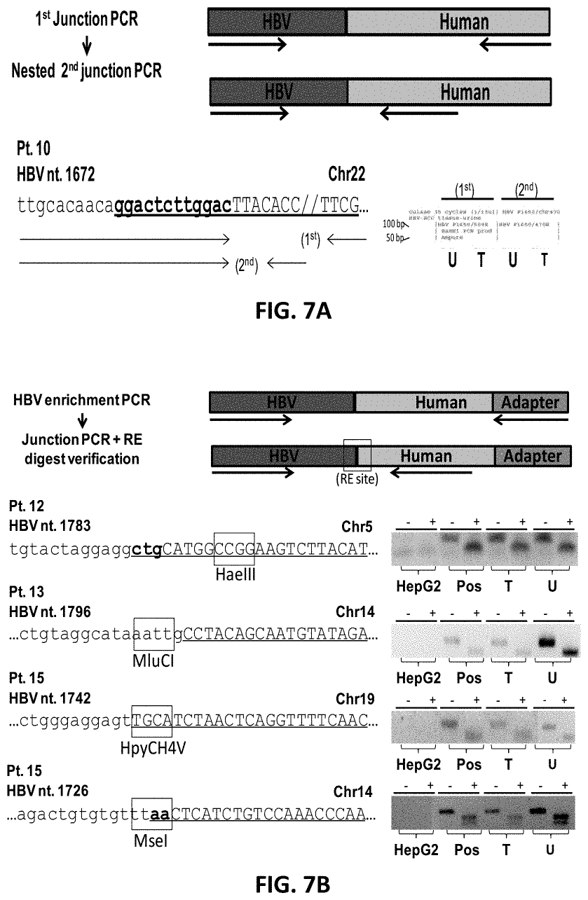

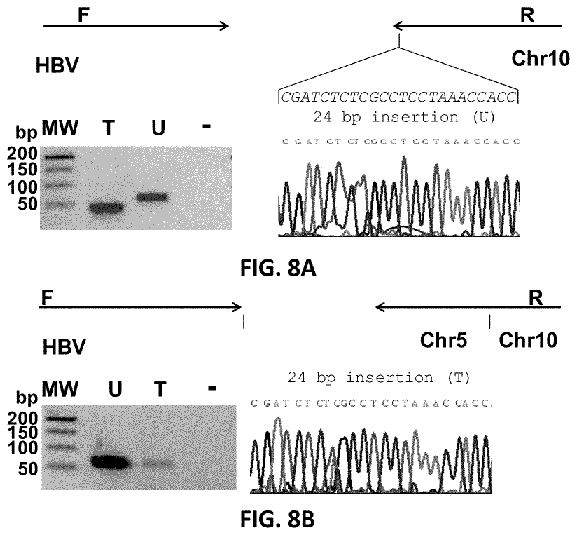

Using the above developed approach, detection of major HBV-JSs derived from HBV-HCC tissue in matched tissue and urine samples was carried out. FIGS. 7A-7B shows the detection of HBV-JSs in matched tissue and urine. FIG. 7A shows a n outline of a PCR based assay where a nested junction PCR approach was used to confirm an HBV-JS from patient 10. HBV and human primers are used to generate the first amplicon (1st) that is followed by a nested primer set to generate a second amplicon (2nd). Both LMW urine DNA (U) and tissue DNA (T) samples were compared. FIG. 7B shows an outline of a PCR based assay where a nested PCR followed by restriction endonuclease (RE) digestion approach was used to confirm each HBV-JS. Patient samples were amplified with HBV and human primers, generating an amplicon with an identifiable RE cleavage site within the amplicon sequence. The amplicon was incubated in the absence (−) or presence (+) of the respective RE. Junction sequence PCR products derived from tissue DNA (Pos) and adapter-ligated HepG2 (HepG2) DNA served as positive and negative controls, respectively. Human and HBV DNA sequences are annotated as described in FIGS. 4A-4B. FIGS. 8A-8B shows the identification of a rearranged HBV-JS in matched tissue and urine DNA. FIG. 8A shows a sequence of the HBV-JS with Chromosome 10 (Chr10) in patient 9 (top). Amplification of this junction sequence using HBV and Chr10 primers resulted in a 24 bp difference between LMW urine DNA (U) and tissue DNA (T) samples (bottom left). The Sanger sequence of inserted 24 bp sequence in urine DNA is depicted in the lower right panel. FIG. 8B shows the detection of the HBV-JS with Chromosome 5 (Chr5) in the corresponding tissue (top). Amplification of tissue DNA of this junction sequence using HBV and chimeric Chr5-Chr10 primers (bottom right) followed by Sanger sequencing confirmed the same inserted 24 bp sequence in tissue DNA (lower right panel). HepG2 DNA was used as the negative control (−). Human and HBV DNA sequences are annotated as described in FIGS. 4A-4B.

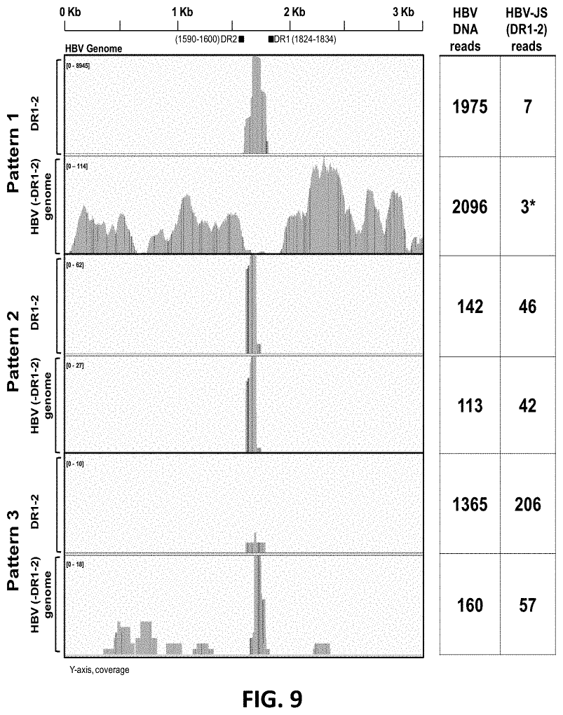

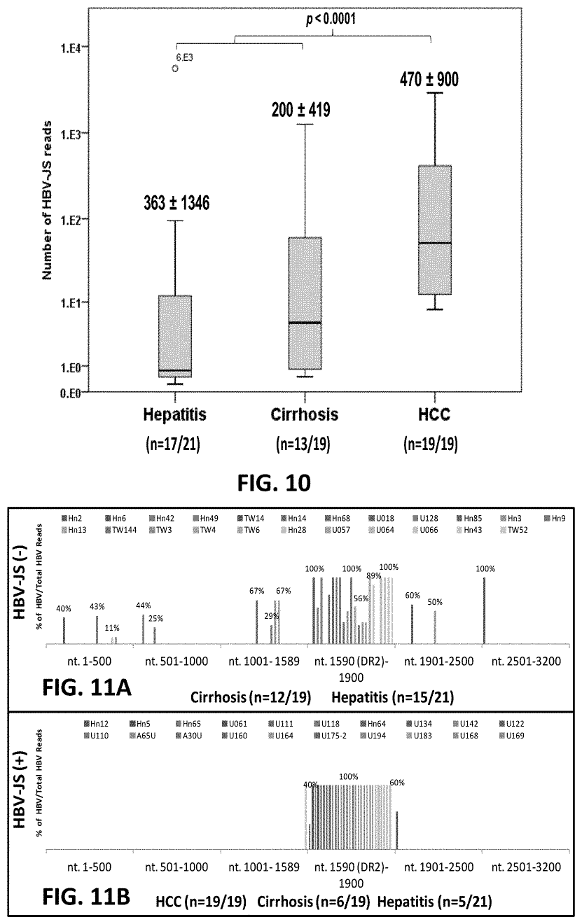

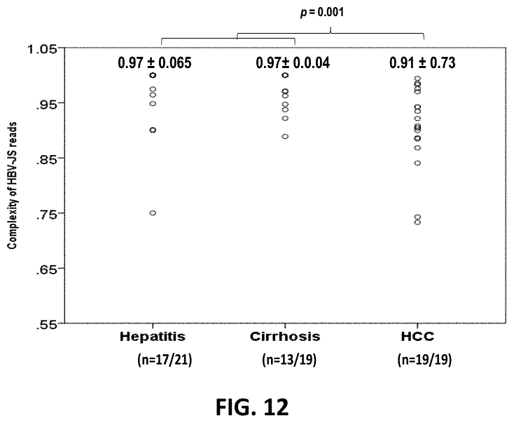

Furthermore, the urine samples of HBV-infected patients are tested for the detection of HBV-JSs. FIG. 9 shows the visualization of HBV DNA reads from HBV DR1-2 (SEQ ID: 29, 31, 33) and HBV (−DR1-2) genome (SEQ ID NO: 6-28, 30, 32, 34-48) enriched NGS. HBV read coverage from HBV DR1-2 and HBV (−DR1-2) genome enriched NGS runs are visualized and are derived from A71K HCC tissue (Pattern 1), A34K HCC urine (Pattern 2), and A34K HCC tissue (Pattern 3). The number of HBV reads and HBV-JS reads located in the DR1-2 region are listed in the left panels next to each visualization. In the figure, “*” denotes HBV-JS reads are not located in the HBV DR1-2 region. The average number of HBV-JS detected in the urine of HBV related hepatitis, cirrhosis, and HCC patients were next compared. As shown in FIG. 10, HBV-JS load in urine of HCC patients is significantly higher compared to non-HCC patients. In the figure, the average number of HBV-JS detected in the urine of HBV related hepatitis, cirrhosis, and HCC patients are graphed for those patients containing HBV-JS. p value was calculated using independent samples Kruskal-Wallis test. FIGS. 11A-11B respectively show a landscape of HBV DNA in urine of HBV-JS (+/−) patients. The regions of the HBV genome categorized as 5 different regions are listed on the x-axis. The % of HBV reads out of total HBV reads are displayed on the y-axis. FIG. 11A shows HBV DNA in urine of cirrhosis and hepatitis patients without HBV-JS. FIG. 11B shows HBV DNA in urine of HCC, cirrhosis, and hepatitis patients with HBV-JS. As shown in the figure, integrated HBV DNA is predominately derived from the DR1-2 region of the HBV genome. A comparison was further carried out between HCC patients compared to non-HCC patients in terms of the HBV-JS complexity in their respective urine samples, and the results are shown in FIG. 12. The average number of HBV-JS detected in the urine of HBV related hepatitis, cirrhosis, and HCC patients are graphed for those patients containing HBV-JS. p value was calculated using independent samples Kruskal-Wallis test. As illustrated in the figure, a reduced HBV-JS complexity is observed in urine of HCC patients compared to non-HCC patients.

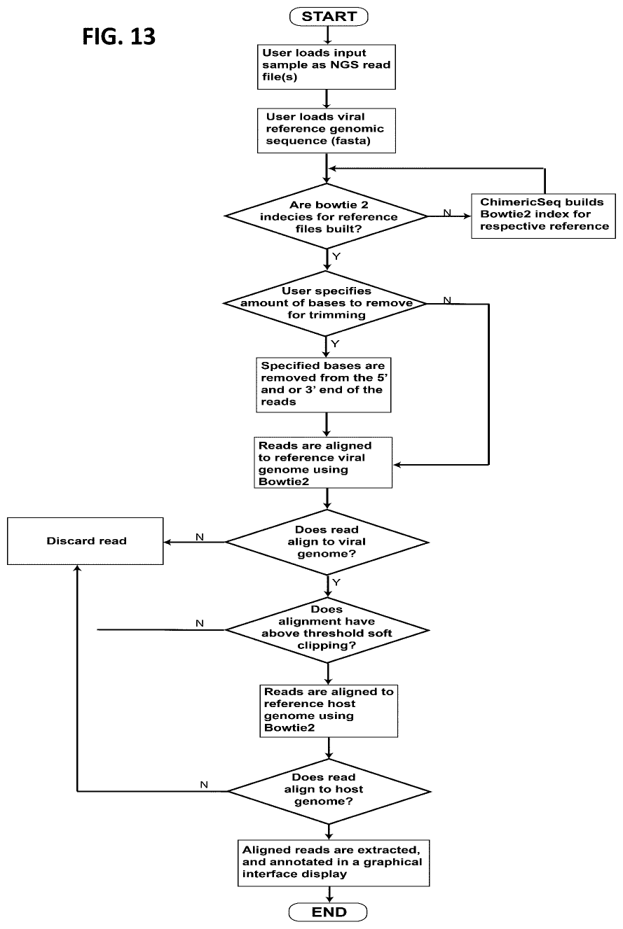

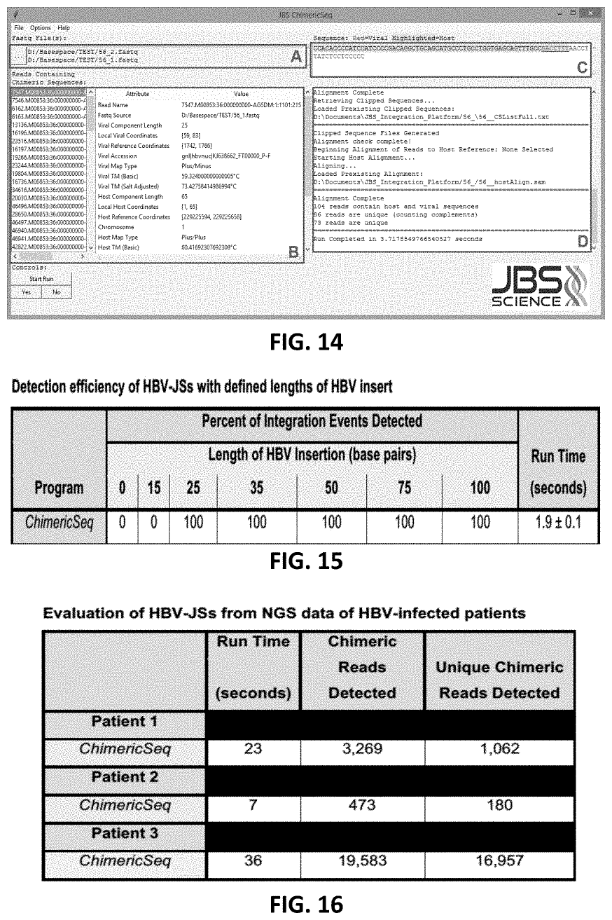

In order to be able to efficiently detect chimeric reads in the nucleotide sequence data, a software package ChimericSeq is developed. FIG. 13 is a schematic overview of the ChimericSeq workflow. As shown, the input NGS reads are manually loaded by the user through a graphical interface, followed by user-determined 5′ and 3′ end trimming as specified. Host and viral genomes along with raw sample data must be identified, if not otherwise already loaded. Next, the identification phase aligns each read to the specified viral genome, extracts these aligned reads, and then aligns the reads to the host genome. The extracted reads are then annotated, analyzed, and presented through the program interface. FIG. 14 further illustrates the ChimericSeq's interactive graphical user interface (GUI). As illustrated, the boxed panel A shows the sequence data of host, virus, and sample NGS reads in fastq/fasta format is loaded into the program, the boxed panel B shows reads containing chimeric sequences are displayed in a column format and the analytical data associated with the selected read is displayed within the table, the boxed panel C shows the selected chimeric read is visualized to highlight different segments and overlap, and the boxed panel D shows the interactive display that communicates questions to the user and also provides logistical information about the run.

In order to evaluate the detection efficiency of integration events with defined lengths of HBV insert, random HBV fragments of specified lengths (0-100 bp) were joined to random human genomic DNA of 100 bp. As shown in FIG. 15, each HBV length category contained reads with HBV inserted in three ways. Within the category, reads were evenly distributed in which HBV was joined at the 5′ terminus, joined at the 3′ terminus, or joined in the center of the 100 bp simulated hg19 read. The total overall percent of chimeric reads detected is listed, as well as the total runtime. 3 independent data sets were acquired to report the average±s.d. To further evaluate ChimericSeq for the capability of detecting integration events from NGS data of HBV-infected patients, NGS data was acquired from three patient tissue samples with known HBV-infection and integration. ChimericSeq was tested for total run time, number of chimeric reads detected, and number of unique chimeric reads (including complements), and the results are shown in FIG. 16. *Indicates the data was not provided as an inherent function of the software, and was manually extracted.

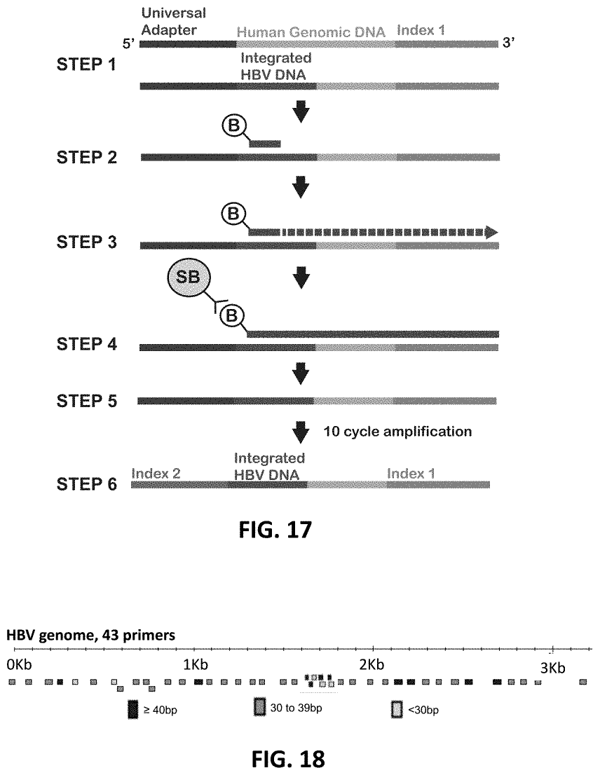

A primer extension capture (PEC) approach for the HBV enrichment has been developed, whose schematic for only one target HBV-host junction sequence (HBV-JS, i.e. a chimeric DNA sequence containing a human genomic DNA and an integrated HBV DNA fragment) is illustrated in FIG. 17. As shown in the figure, in step 1, library preparation of isolated DNA from a biological sample of a patient with HBV-associated disease gives rise to sequences containing only genomic DNA or sequences containing HBV DNA integrated into genomic DNA. Each such sequence is flanked by a pair of adaptors ligated to the two ends (i.e. a universal adaptor, and an adaptor containing Index 1). In step 2, a biotinylated primer for HBV (shown as a short primer labeled with a biotin moiety, i.e. encircled B in the figure, at a 5′-end thereof), which is designed to have a sequence that is complementary with the HBV DNA in the targeting HBV sequence, is annealed with the target HBV sequence obtained from step 1. In step 3, the annealed primer is extended by amplification, creating a very high binding affinity. In step 4, magnetic streptavidin-coated beads are used to capture the primer-extended DNA, while the unbound DNAs are washed away. In step 5, DNAs that are captured in step 4 is eluted from the biotinylated beads by NaOH, giving rise to ssDNAs having target HBV sequences. In step 6, the eluted DNA molecules are further amplified by e.g. 10 cycles, to thereby also add an Index 2. After step 6, the enriched and amplified DNA sequences can then undergo sequencing analysis, or other treatments, such as another round of same enrichment from step 1 through step 6.

It is noted that FIG. 17 only illustrates the enrichment of target HBV sequences by means of one single biotinylated HBV primer (i.e. it targets only one single HBV fragment). In order to realize the simultaneous enrichment of a variety of HBV sequences having a different integrated HBV DNA sequences, a plurality of biotinylated HBV primers can be designed to target the various region of the HBV genome. In the above Examples 1-2, an HBV probe panel, consisting of 43 HBV biotinylated short probes which respectively target the different genomic regions of the genotypes B and C of the HBV genome (shown in FIG. 18), was originally utilized.

With a purpose to provide a broader coverage, an optimized probe panel is further developed, which includes a total of 127 probes (Table 3) covering the most frequent four genotypes (A-D) of HBV and covering the entire HBV genome, is further developed. Briefly, to design an HBV probe panel with high specificity and sensitivity for application in an HBV primer-extension capture (PEC) approach, a human micro-homology analysis was first performed to identify regions within the HBV genome that are highly homologous to the human genome. The analysis was done by performing an NCBI BLAST query to the human genome for every 50 bp increments of HBV DNA along the entire 3.2 kb genome. The analysis uncovered 142 human micro-homologous stretches of HBV DNA ranging from 10-30 bp (average size of 19.6 bp) with melting temperatures (Tm) as high as 65° C. A total of 127 HBV probes were next designed to target the antisense strand along the entire HBV genome for genotypes A-D that avoided these human micro-homologous stretches. When it was not possible to avoid human micro-homologous stretches containing a Tm of 55° C. or less, the HBV primer was designed to target the HBV sense strand to ensure full HBV genome coverage during the enrichment.

TABLE 3
Primer lists in the optimized HBV probe panel.

| Primer | Primer | | |
|---|---|---|---|
| Name | Region | Sequence | SEQ ID NOS |
| 1 | 3-34 F | ACAACATTCCACCAARCTCTKCTAGATCCC | SEQ ID NO: 49 |
| 2 | 95-126 R | AAGATTGACGATATGGWTGAGGCAGTAGTCGGAACAG | SEQ ID NO: 50 |
| | | GG | |
| 3 | 201-240 R | GGTATTGTGAGGATTTTTGTCAACAAGAAAAACCCCGC | SEQ ID NO: 51 |
| | | CT | |
| 4 | 270-299 R | GACACACGGGTGYTCCCCCTAGAAAATTG | SEQ ID NO: 52 |
| 5 | 382-356 R | ACACATCCAGCGATARCCAGGACAAYTRGG | SEQ ID NO: 53 |
| 6 | 456-486 F | AGGTATGTTGCCCGTTTGTCCTCTAMTTCC | SEQ ID NO: 54 |
| 7 | 570-597 F | TACAAAACCTWCGGACGGAAAYTGCAC | SEQ ID NO: 55 |
| 8 | 605-635 F | CCCATCCCATCATCYTGGGCTTTCGCAARA | SEQ ID NO: 56 |
| 9 | 693-725 R | AAACAGTGGGGGAAAGCCCTACGAACCACTG | SEQ ID NO: 57 |
| 10 | 749-780 F | GGTACTGGGGGCCAAGTCTGTACAACATCTT | SEQ ID NO: 58 |
| 11 | 781-810 F | GAGTCCCTTTATRCCGCTRTTACCAATTTTCTTTTGTCTT | SEQ ID NO: 59 |
| 12 | 871-900 F | CCCTTAACTTCATGGGATATGTAATTGGRAGTTGG | SEQ ID NO: 60 |
| 13 | 951-980R | TTCCAATCAATAGGYCTGTTTACAGGCAGTTTCCKAAA | SEQ ID NO: 61 |
| | | AC | |
| 14 | 1033-1077F | CAATGTGGMTATCCTGCTTTRATGCCTTTATATGCATGT | SEQ ID NO: 62 |
| | | ATACAA | |
| 15 | 1101-1130 R | TGTTTACACAGAAAGGCCTTGTAAGTTGGC | SEQ ID NO: 63 |
| 16 | 1183-1212 R | GCCCCAACCCGTGGGGGTTGCGTCAGCAAA | SEQ ID NO: 64 |
| 17 | 1261-1290 R | AGCKGCTAGGAGTTCCGCAGTATGGATCGG | SEQ ID NO: 65 |
| 18 | 1342-1371 F | GTTGTCCTCTCTCGGAAATACACCGCCTTT | SEQ ID NO: 66 |
| 19 | 1395-1424 F | CAACTGGATCCTGCGCGGGACGTCCTTTGT | SEQ ID NO: 67 |
| 20 | 1513-1542 F | CCGACCACGGGGCGCACCTCTCTTTACGCG | SEQ ID NO: 68 |
| 21 | 1575-1604 R | ACGTGCAGAGGTGAAGCGAAGTGCACACGG | SEQ ID NO: 69 |
| 22 | 1613-1629 F | GACCACCGTGAACGCCC | SEQ ID NO: 70 |
| 23 | 1633-1653 F | AGGTCTTGCCCAAGGTCTTAC | SEQ ID NO: 71 |
| 24 | 1650-1671 F | TTGCACAACAGGACTCTTGGAC | SEQ ID NO: 72 |
| 25 | 1686-1709 F | AACGACCCGACCTTGAGGCATACTTC | SEQ ID NO: 73 |
| 26 | 1741-1767 F | TRGGGGAGGAGATAAGGTTAAAGGTC | SEQ ID NO: 74 |
| 27 | HBV_F_1650_ | TTACATAAGAGGACTCTTGGAC | SEQ ID NO: 75 |
| | 1672_1 | | |
| 28 | HBV_F_1741_ | TRGGGGAGGAGATTAGGTTAAAGGTC | SEQ ID NO: 76 |
| | 1767_1 | | |
| 29 | HBV_F_1741_ | TGGGGGAGGAGATTAGGTTAATGATC | SEQ ID NO: 77 |
| | 1767_DM | | |
| 30 | 1828-1862 F | CCTCTGCCTAATCATCTCATGTTCATGTCCTACTG | SEQ ID NO: 78 |
| 31 | 1896-1930 F | GGGGCATGGACATTGACCCSTATAAAGAATTTGGA | SEQ ID NO: 79 |
| 32 | 1997-2026 F | ACCGCCTCTGCTCTGTATCGGGAGGCCTTA | SEQ ID NO: 80 |
| 33 | 2081-2110 F | TGTTGGGGTGAGTTGATGAATCTRGCCACC | SEQ ID NO: 81 |
| 34 | 2146-2190 R | ATTTTTAGGCCCATATTAACRTTGACATAGCTGACTACT | SEQ ID NO: 82 |
| | | AATTCC | |
| 35 | 2221-2260R | CACCAAATAYTCAAGRACAGTTTCTCTTCCAAAAGTAA | SEQ ID NO: 83 |
| | | GR | |
| 36 | 2305-2340 R | GTAGTTTCCGGAAGTGTTGATAAGATAGGGGCATTT | SEQ ID NO: 84 |
| 37 | 2380-2409 F | TCCCTCGCCTCGCAGACGAAGGTCTCAATC | SEQ ID NO: 85 |
| 38 | 2466-2502 R | TAGAAGAATAAAGCCCAGTAAAGTTTCCCACCTTATG | SEQ ID NO: 86 |
| 39 | 2541-2580 F | TTTCCTSACATTCATCTACAGGAGGACATTRTTRATAGA | SEQ ID NO: 87 |
| | | T | |
| 40 | 2697-2740 F | CCGTATTATCCWGARCATGCAGTTAATCATTACTTCAA | SEQ ID NO: 88 |
| | | AACTAG | |
| 41 | 2783-2812 R | CAAAATGAGGCGCTRCGTGTAGTYTCTCTY | SEQ ID NO: 89 |
| 42 | 2851-2880 F | GGAGGTTGGTCTTCCAAACCTCGACAAGGC | SEQ ID NO: 90 |
| 43 | 2931-2960 F | CCAGTTGGACCCTGCRTTCRRAGCCAACTC | SEQ ID NO: 91 |
| 44 | 3181-3215 F | TCATCCTCAGGCCATGCAGTGGAA | SEQ ID NO: 92 |
| 45 | HBV_2146_ | AACTTTAGGCCCATATTAGTRTTGACATAGCTGACTACT | SEQ ID NO: 93 |
| | 2190_D_RC | AGGTCY | |
| 46 | HBV_95_126_ | AAGATTGACGATATGGGAGAGGCAGTAGTCGGAACAG | SEQ ID NO: 94 |
| | RC_C | GG | |
| 47 | HBV_95_126_ | AAGATTGACGATATGGMAGAGGCAGTATTCTGARCAG | SEQ ID NO: 95 |
| | RC_B | GG | |
| 48 | HBV_95_126_ | AAGATTGACGATAWGGGAGAGGCAGTAGTCRGAACAG | SEQ ID NO: 96 |
| | RC_A | GG | |
| 49 | HBV_3_34_A | ACARCCTTCCACCAARCTCTKCAAGATCCC | SEQ ID NO: 97 |
| | B_D | | |
| 50 | HBV_50_80_ | TATTTYCCTGCTGGTGGCTCCAGTTCMGGAA | SEQ ID NO: 98 |
| | All | | |
| 51 | HBV_340_ | ACATCCAGCGATAACCAGGACAAGTTGGAGGACARGA | SEQ ID NO: 99 |
| | 380_RC_A_D | GGTT | |
| 52 | HBV_340_ | ACATCCAGCGATARCCAGGACAARTTGGAGGACAASAG | SEQ ID NO: 100 |
| | 380_RC_B_C | GTT | |
| 53 | HBV_1997_ | ACCGCCTCAGCTCTGTATCGGGAGGCCTTA | SEQ ID NO: 101 |
| | 2026_A_D | | |
| 54 | HBV_520_ | AGAGGTTCCTTGAGCAGGAATCGTGCAGGTT | SEQ ID NO: 102 |
| | 550_All_RC | | |
| 55 | HBV_390_ | AGCAGCAGGATGAAGAGGAAKATGATAAAAC | SEQ ID NO: 103 |
| | 420_RC_All | | |
| 56 | HBV_1220_ | AGGAGCCACAAAGGTTCCACGCATGCGCYGATGGCCY | SEQ ID NO: 104 |
| | 1260_B_C_RC | A | |
| 57 | HBV_1220_ | AGGAGCCASAAAGGTTCCACGCATGCGCCGATGGCCYA | SEQ ID NO: 105 |
| | 1260_A_D_RC | | |
| 58 | HBV_305_ | AGTGACTGGAGATTTGGGACTGCGAATTTTG | SEQ ID NO: 106 |
| | 335_RC_B | | |
| 59 | HBV_305_ | AGTGATTGGAGGTTGGGGACTGCGAATTTTG | SEQ ID NO: 107 |
| | 335_RC_A_D_ | | |
| | C | | |
| 60 | HBV_2146_ | ATCTTTAGGCCCATATTAGTRTTGACATAGTTGACTACT | SEQ ID NO: 108 |
| | 2190_A_RC | AGATCC | |
| 61 | HBV_640_ | ATGGGAGTGGGCCTCAGYCCGTTTCTCCTGGCTCAGTTT | SEQ ID NO: 109 |
| | 680_All | AC | |
| 62 | HBV_1033_ | CAATGTGGMTATCCTGCYTTRATGCCTTTRTATGCATGT | SEQ ID NO: 110 |
| | 1077_B_C | ATACAA | |
| 63 | HBV_1033_ | CAATGTGGWTATCCTGCTTTRATGCCYTTGTATGCATG | SEQ ID NO: 111 |
| | 1077_A_D | TATTCAA | |
| 64 | HBV_170_ | CAGGAYTCCTAGGACCCCTGCTCGTGTTA | SEQ ID NO: 112 |
| | 200_All | | |
| 65 | HBV_2931_ | CCAGTTGGATCCAGCCTTCAGAGCAAACAC | SEQ ID NO: 113 |
| | 2960_D | | |
| 66 | HBV_605_ | CCCATCCCATCATCYTGGGCTTTCGGAAAA | SEQ ID NO: 114 |
| | 635_D | | |
| 67 | HBV_871_ | CCCTAAAYTTCATGGGYTATGTAATTGGRAGTTGG | SEQ ID NO: 115 |
| | 900_A_D-1 | | |
| 68 | HBV_910_ | CCGCAAGATCAYATYRTACAAAAAATCAAGG | SEQ ID NO: 116 |
| | 940_A_D | | |
| 69 | HBV_910_ | CCRCARGAACATATTGTACAAAAAATCAARC | SEQ ID NO: 117 |
| | 940_B_C | | |
| 70 | HBV_1828_ | CCTCTGCCTAATCATCTCWTGTTCATGTCCTACTG | SEQ ID NO: 118 |
| | 1862_B_C_D | | |
| 71 | HBV_2697_ | CCTTATTATCCAGAGCATGTAGTTAATCATTACTTCCA | SEQ ID NO: 119 |
| | 2740_B | GACRAG | |
| 72 | HBV_2697_ | CCTTATTATCCWGARCATSTAGTTAATCATTACTTCCAA | SEQ ID NO: 120 |
| | 2740_A_D | ACYAG | |
| 73 | HBV_1300_ | CGCAGCCGGTCTGGAGCGAAACTCATCGGAACTGAC | SEQ ID NO: 121 |
| | 1334_A | | |
| 74 | HBV_1300_ | CGCAGCCGGTCTGGAGCGAAACTTATCGGAACCGAC | SEQ ID NO: 122 |
| | 1334_C | | |
| 75 | HBV_1300_ | CGCAGCMGGTCTGGAGCGAAAATTATCGGAACTGAY | SEQ ID NO: 123 |
| | 1334_B_D | | |
| 76 | HBV_1135_ | CTAAACCTTTACCCCGTTGCCCGGCAACGGTCAGGT | SEQ ID NO: 124 |
| | 1170_A_D | | |
| 77 | HBV_1135- | CTAAACCTTTACCCCGTTGCTCGGCAACGGCCAGGT | SEQ ID NO: 125 |
| | 1170_B | | |
| 78 | HBV_1135_ | CTAAACCTTTACCCCGTTGCTCGGCAACGGTCAGGT | SEQ ID NO: 126 |
| | 1170_C | | |
| 79 | HBV_1828_ | CTCTGCCTAATCATCTCTTGTACATGTCCTACTK | SEQ ID NO: 127 |
| | 1862_A | | |
| 80 | HBV_2110_ | CTGGGTGGGWARTAATTTGGAAGAYCCAGCR | SEQ ID NO: 128 |
| | 2140_All | | |
| 81 | HBV_871_ | CTYTAAATTTCATGGGYTATGTCATTGGRAGTTAT | SEQ ID NO: 129 |
| | 900_A_D-2 | | |
| 82 | HBV_270_ | GACACRCGGKWGYTCCCCCTAGAAAATTG | SEQ ID NO: 130 |
| | 299_RC_All | | |
| 83 | HBV_130_ | GAGGACTGGGGACCCTGCRCCGAACATGGAG | SEQ ID NO: 131 |
| | 160_A_B_C | | |
| 84 | HBV_130_ | GAGGATTGGGGACCCTGCGCTGAACATGGAG | SEQ ID NO: 132 |
| | 160_D | | |
| 85 | HBV_781_ | GAGTCCCTTTWTRCCGCTRTTACCAATTTTCTTTTGTCT | SEQ ID NO: 133 |
| | 810_All | T | |
| 86 | HBV_1183_ | GCCCCARCCAGTGGGGGTTGCGTCAGCAAA | SEQ ID NO: 134 |
| | 1212_All_RC | | |
| 87 | HBV_2410_ | GCCGCGTCGCAGAAGATCTCAATCTCGGGAA | SEQ ID NO: 135 |
| | 2440_All | | |
| 88 | HBV_1780_ | GCTGTAGGCATAAATTGGTCTGCGCACCAGCACCAT | SEQ ID NO: 136 |
| | 1810_A_D | | |
| 89 | HBV_1780_ | GCTGTAGGCATAAATTGGTCTGTTCACCAGCACCAT | SEQ ID NO: 137 |
| | 1810_B_C | | |
| 90 | HBV_990_ | GGGGCAGCAAAGCCCAAAAGACCCACAATTCKTTGA | SEQ ID NO: 138 |
| | 1025_RC_All | | |
| 91 | HBV_1896_ | GGGGCATGGACATTGACCCKTATAAAGAATTTGGA | SEQ ID NO: 139 |
| | 1930_All | | |
| 92 | HBV_749_ | GGTATTGGGGGCCAAGTCTGTACARCATCTT | SEQ ID NO: 140 |
| | 780_All | | |
| 93 | HBV_201_ | GGTATTGTGAGGATTYTTGTCAACAAGAAAAACCCCGC | SEQ ID NO: 141 |
| | 240_RC_All | CT | |
| 94 | HBV_1342_ | GTYGTCCTCTCCCGSAAATATACAGCGTTT | SEQ ID NO: 142 |
| | 1371_A_B_D | | |
| 95 | HBV-570_ | TACCAAACCTTCGGACGGAAAYTGCAC | SEQ ID NO: 143 |
| | 597_D | | |
| 96 | HBV_2466_ | TAGAAGAATAAAGCCCMGTAAAGTTTCCCACCTTATG | SEQ ID NO: 144 |
| | 2502_All_RC | | |
| 97 | HBV_3181_ | TCATCCTCAGGCCATGCAGTGG | SEQ ID NO: 145 |
| | 3215_D | | |
| 98 | HBV_2081_ | TGCTGGGGGGARTTGATGACTCTRGCTACC | SEQ ID NO: 146 |
| | 2110_A_B_D | | |
| 99 | HBV_1101_ | TGTTTACAYAGAAAGGCCTTGTAAGTTGGC | SEQ ID NO: 147 |
| | 1130_All_RC | | |
| 100 | HBV_951_ | TTCCAATCAATAGGTCTATTTACAGGAAGTTTTCKAAA | SEQ ID NO: 148 |
| | 980_C_RC | AC | |
| 101 | HBV_951_ | TTCCAATCAATAGGYCTGTTAACAGGAAGTTTTCKAAA | SEQ ID NO: 149 |
| | 980_A_D_RC | AC | |
| 102 | HBV_820_ | TTGGGTATACATTTGAACCCTAACAAAACCAAACGA | SEQ ID NO: 150 |
| | 855_A_D | | |
| 103 | HBV_820_ | TTGGGTATACATTTGAACCCTAATAAAACAAAAACGT | SEQ ID NO: 151 |
| | 855_B | | |
| 104 | HBV_820_ | TTGGGTATACATTTGAACCCTAATAAAACCAAACGT | SEQ ID NO: 152 |
| | 855_C | | |
| 105 | HBV_1650_ | TTRCACAAGAGGACTCTTGGAC | SEQ ID NO: 153 |
| | 1671_All | | |
| 106 | HBV_2541_ | TTTCCTAARATTCATTTACAWGAGGACATTRTTAATAG | SEQ ID NO: 154 |
| | 2580_A | AT | |
| 107 | HBV_2541_ | TTTCCTAATATACATTTACAGCAGGACATTATCAAAAA | SEQ ID NO: 155 |
| | 2580_D | AT | |
| 108 | HBV_1480_ | CTCTATCGTCCCCTTCTTCATCTGCCGTTCC | SEQ ID NO: 156 |
| | 1510_A_D | | |
| 109 | HBV_1480_ | CTCTACCGYCCSCTTCTTCATCTGCCGTWCC | SEQ ID NO: 157 |
| | 1510_B_C | | |
| 110 | HBV_1940_ | GGAAAGAAGTCAGAAGGCAAAAACGAGAGTAACTC | SEQ ID NO: 158 |
| | 1970_RC_A_D | | |
| 111 | HBV_1940_ | GGAAAGAAGTCAGAAGGCAAAAAAGAGAGTAACTC | SEQ ID NO: 159 |
| | 1970_RC_B_C | | |
| 112 | HBV_2030_ | TCTCCTGARCATTGYTCACCTCACCATACRG | SEQ ID NO: 160 |
| | 2060_A_B | | |
| 113 | HBV_2030_ | TCTCCTGAGCATTGTTCACCTCACCATACTG | SEQ ID NO: 161 |
| | 2060_D | | |
| 114 | HBV_2030_ | TCTCCGGAACATTGTTCACCTCACCATACAG | SEQ ID NO: 162 |
| | 2060_C | | |
| 115 | HBV_2510_ | TATCTTTAATCCTGAATGGCAAACTC | SEQ ID NO: 163 |
| | 2535_A | | |
| 116 | HBV_2510_ | TGTCTTTAATCCTCATTGGAAAACAC | SEQ ID NO: 164 |
| | 2535_D | | |
| 117 | HBV_2510_ | TGTCTTTAATCCTGARTGGCAAACTC | SEQ ID NO: 165 |
| | 2535_B_C | | |
| 118 | 2620_2650_ | AACCTAGCAGGCATAATCAATTKCARTCTTC | SEQ ID NO: 166 |
| | RC_A_D | | |
| 119 | HBV_2620_ | AACCTAGCAGGCATAATTAATTTTAGTCTCC | SEQ ID NO: 167 |
| | 2650_RC_B | | |
| 120 | HBV_2620_ | AACCTAGCAGGCATAATTAATTTTAATCTCC | SEQ ID NO: 168 |
| | 2650_RC_C | | |
| 121 | HBV_2822_ | CATGCTGTAGCTCTTGTTCCCAAGAATAT | SEQ ID NO: 169 |
| | 2850_RC_All | | |
| 122 | HBV_2890_ | AATCTTTCTGTYCCCAATCCTCTGGGATTCTTTCCCGAT | SEQ ID NO: 170 |
| | 2930_A_B_C | CA | |
| 123 | HBV_2890_ | AATCTTTCCACCAGCAATCCTCTGGGATTCTTTCCCGAC | SEQ ID NO: 171 |
| | 2930_D | CA | |
| 124 | HBV_3010_ | CCAACAAGGTAGGAGYKGGAGCATTCGGGC | SEQ ID NO: 172 |
| | 3040_All | | |
| 125 | HBV_3060_ | ATATGCCCTGAGCCTGAGGGCTCCACCCCAAAACACCT | SEQ ID NO: 173 |
| | 3100_RC_A | CCG | |
| 126 | HBV_3060_ | GTATGCCCTGAGCCTGAGGGCTCCACCCCAAAAGKCCY | SEQ ID NO: 174 |
| | 3100_RC_D_B | CCR | |
| 127 | HBV_3060_ | ATATGCCCTGAGCCTGAGGGCTCCACCCCAAAAGACCG | SEQ ID NO: 175 |
| | 3100_RC_C | CCG | |

*All primers are labelled with a 5′ biotin modification. ′R′ base denotes redundant A + G base. ′Y′ base denotes redundant C + T base. ′W′ base denotes redundant A + T base. ′S′ base denotes G + C base. ′K′ base denotes redundant G + T base. ′M′ base denotes redundant A + C base.

In Examples 1-3, all the HBV enrichment experiments, if any, were performed based on the double-stranded DNA (dsDNA) library construction. Out of curiosity, a similar enrichment experiment based on the single-stranded DNA (ssDNA) library construction, was also carried out, and compared with a parallel enrichment experiment based on dsDNA library construction from the same biological sample. Briefly, cell-free DNA (cfDNA) samples isolated form liquid biopsy specimens (urine) from different patient samples, was utilized for both ssDNA and dsDNA library construction, which then underwent HBV enrichment, and NGS sequencing analysis. For ssDNA library construction, the ClaretBio SRSLY™ PicoPlus DNA NGS Library Preparation Dual UMI Index kit was utilized where a critical DNA denaturing step is performed as the initial step. All other subsequent steps were performed in accordance with the manufacturer's protocol. For library construction of double-stranded DNA, the Takara SMARTer® ThruPLEX® Tag-seq kit was utilized and performed according to the manufacturer's protocol.

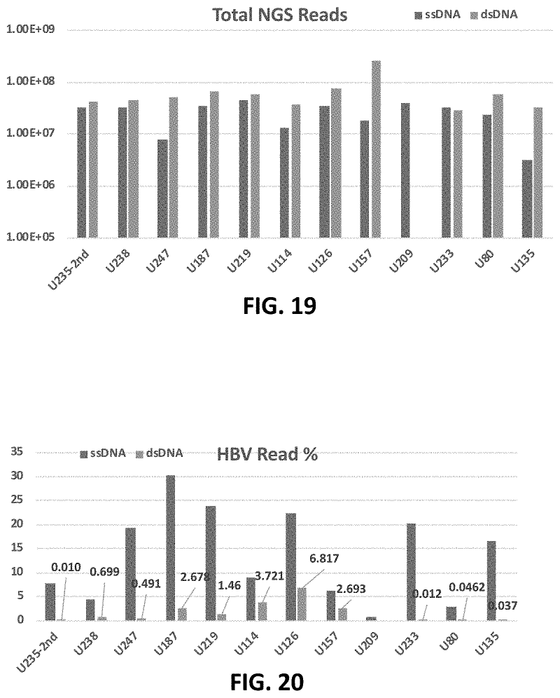

Unexpectedly, a significantly improved HBV (on-target) enrichment was observed in urine samples utilizing single-strand DNA library construction compared with the same urine samples utilizing double-strand DNA library construction (Table 4). While both methods have obtained a similar level of total NGS reads (FIG. 19), the “HBV reads %” is much more pronounced in the ssDNA library group than in the dsDNA library group (FIG. 20), and importantly, the total number of HBV-JS reads is much higher in the ssDNA library group than in the dsDNA library group (Table 4). Thus it appears that ssDNA library construction method can provide more HBV DNA containing templates, thus a better HBV-JS enrichment and identification result if working with a biological sample such as a urine sample.

TABLE 4
Comparison of HBV-targeted enriched NGS results between ssDNA and dsDNA library
construction methods over the same urine samples.

| | | Single Strand Method | | | | Double Strand Method | | | |
|---|---|---|---|---|---|---|---|---|---|
| | | Total | | HBV | # | Total | | HBV | # |
| Disease- | Patient | NGS | HBV | Reads | HBV-JS | NGS | HBV | Reads | HBV-JS |
| Type | Urine | Reads | Reads | % | Reads | Reads | Reads | % | Reads |
| HCC | U235-2nd | 3.20E+07 | 1.95E+06 | 7.781 | 2955 | 4.14E+07 | 4.09E+03 | 0.010 | 50 |
| HCC | U238 | 3.33E+07 | 1.44E+06 | 4.340 | 1153 | 4.58E+07 | 3.20E+05 | 0.699 | 77 |
| HCC | U247 | 8.00E+06 | 1.56E+06 | 19.485 | 5718 | 5.05E+07 | 2.48E+05 | 0.491 | 207 |
| Post-HCC | U187 | 3.55E+07 | 1.08E+07 | 30.272 | 2295 | 6.70E+07 | 1.79E+06 | 2.678 | 352 |
| Post-HCC | U219 | 4.55E+07 | 1.09E+07 | 23.892 | 9833 | 5.81E+07 | 8.46E+05 | 1.46 | 101 |
| Cirrhosis | U114 | 1.30E+07 | 1.18E+06 | 9.083 | 1695 | 3.75E+07 | 1.39E+06 | 3.721 | 145 |
| Cirrhosis | U126 | 3.48E+07 | 7.84E+06 | 22.492 | 2780 | 7.48E+07 | 5.10E+06 | 6.817 | 307 |
| Cirrhosis | U157 | 1.78E+07 | 1.13E+06 | 6.349 | 513 | 252816308 | 6809276 | 2.693 | 117 |
| Cirrhosis | U233 | 3.24E+07 | 6.61E+06 | 20.411 | 1657 | 2.95E+07 | 3.46E+03 | 0.012 | 62 |
| Hepatitis | U80 | 2.36E+07 | 6.98E+05 | 2.959 | 2128 | 5.69E+07 | 2.63E+04 | 0.0462 | 134 |
| Hepatitis | U135 | 3.12E+06 | 5.22E+05 | 16.704 | 2828 | 3.37E+07 | 1.24E+04 | 0.037 | 33 |

In order to evaluate the performance of the optimized HBV probe panel (n=127, shown in Table 10) relative to the initial HBV probe panel (n=43, shown in Table 1), enrichment analysis was carried out using reconstituted PLC HCC cell-line DNA containing known integrated HBV sequences, where normal DNA samples containing 1%, 0.5%, and 0.1% PLC genomic DNAs were compared for sensitivity and specificity evaluation, and the results are shown in Table 5. After two sequential primer-extension capture (PEC), both panels demonstrate ˜10⁵-fold enrichment compared to whole genome sequence of 100% PLC (no enrichment).

TABLE 5
Assay assessment of initial vs optimized probe panel.

| Sample | Description | Total NGS Reads | HBV Reads % |
|---|---|---|---|
| PLC 1% | Optimized panel | 2.99E+07 | 0.500 |
| | | 1.85E+08 | 0.620 |
| | | 2.02E+08 | 0.573 |
| | Initial panel | 2.19E+08 | 0.528 |
| | | 2.34E+08 | 0.496 |
| | | 2.55E+08 | 0.457 |
| PLC 0.5% | Optimized panel | 2.19E+08 | 0.528 |
| | | 4.05E+08 | 0.293 |
| | | 4.19E+08 | 0.285 |
| | Initial panel | 2.71E+08 | 0.431 |
| | | 2.87E+08 | 0.407 |
| | | 3.04E+08 | 0.385 |
| PLC 0.1% | Optimized panel | 4.34E+08 | 0.276 |
| | | 4.50E+08 | 0.266 |
| | | 4.65E+08 | 0.257 |
| | Initial panel | 3.21E+08 | 0.364 |
| | | 3.36E+08 | 0.348 |
| | | 3.56E+08 | 0.329 |

The optimized HBV panel was also examined for its performance in detecting known HBV-junctions (such as HBV junction at TERT, CCDCl57 and MVK). As shown in Table 6, the optimized panel showed a better performance, and can detect additional junction reads compared to the initial panel when the number of NGS reads are similar.

TABLE 6
Detection of known HBV-junctions using optimized vs initial HBV panel.

| Sample | Description | # HBV-JS Reads | TERT | CCDC57 | MVK |
|---|---|---|---|---|---|
| PLC 1% | Optimized panel | 121 | 3 | 4 | 7 |
| | | 160 | 0 | 19 | 9 |
| | | 144 | 0 | 18 | 8 |
| | Initial panel | 20 | 1 | 3 | 0 |
| | | 21 | 0 | 0 | 0 |
| | | 70 | 0 | 6 | 30 |
| PLC 0.5% | Optimized panel | 89 | 0 | 4 | 0 |
| | | 104 | 0 | 12 | 5 |
| | | 45 | 0 | 3 | 2 |
| | Initial panel | 3 | 0 | 0 | 0 |
| | | 2 | 1 | 0 | 0 |
| | | 16 | 3 | 0 | 0 |
| PLC 0.1% | Optimized panel | 22 | 0 | 4 | 0 |
| | | 23 | 0 | 0 | 2 |
| | | 13 | 0 | 0 | 0 |
| | Initial panel | 2 | 0 | 0 | 0 |
| | | 3 | 1 | 0 | 0 |
| | | 2 | 0 | 0 | 0 |

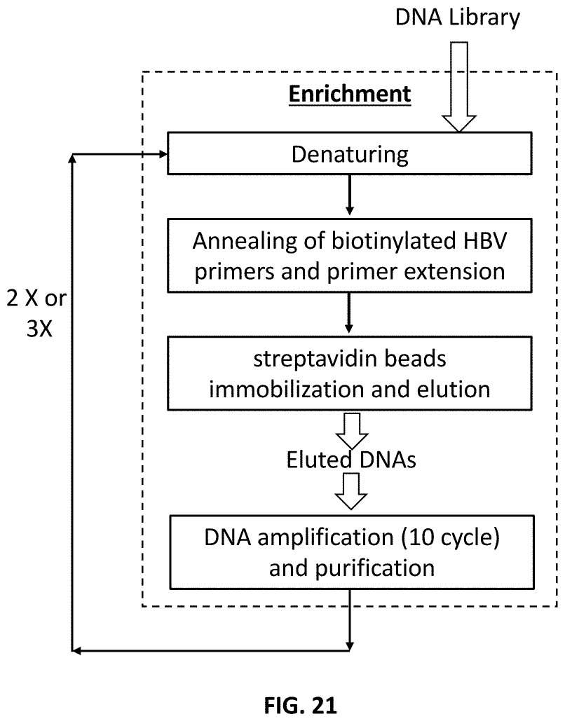

In order to further evaluate whether an increased number of PEC enrichment can improve the enrichment result, a comparison experiment was carried out, which compare the two sequential PEC enrichment with three sequential PEC enrichment. Briefly, the workflow of a sequential PEC enrichment is illustrated in FIG. 21. Specifically, a multiplex biotin HBV primer extension reaction was performed using library DNA in a reaction containing 1× Herculase II Buffer, 250 μM dNTP, and 25 pmol of each 127 biotinylated HBV primers and 0.25 pmol of adapter blockers (shown below, where “-PH” denotes a 3′ phosphorphylation of the oligo, and “+” denotes a modified locked nucleic acid nucleotide).

P5 trunc block
(SEQ ID NO: 176)
GTGTAGATCTCGGTGGTCGCCGTATCATT-PH
P7 trunc block
(SEQ ID NO: 177)
CAAGCAGAA+GACGGCATACGA+GAT-PH

First, reaction containing buffer, blockers, dNTP and library DNA was incubated at 95° C. for 5 mins to denature double-strand library DNA and facilitate binding of adapter blockers to prevent daisy chaining during enrichment. Next, the reaction was held at 72° C. for 5 mins before adding the biotinylated HBV primer mix to the reaction. The entire reaction was incubated at 60° C. for 1 hr. Lastly, 0.1 μl of heat inactivated Herculase II Fusion polymerase was added to each reaction and incubated at 72° C. for 90 s. The captured DNA was collected by using hydrophilic streptavidin magnetic beads (New England Biolabs, Ipswich, Mass.), washed twice at 55° C. using 5 mM TrisHCl pH 7.5, 0.5 mM EDTA, 1M NaCl buffer. Captured library DNA was eluted using 10 μl 0.1N NaOH and neutralized with 40 μl 1M Trish-HCl pH7.5. Prior to post-enrichment amplification, eluted library DNA was purified using 1.8× AMPure XP beads. Library DNA amplification post-enrichment utilized 1× Herculase II Buffer, 250 μM dNTP, and 30 pmol of P5/P7 Illumina adapter primers, and 0.3 μl of Herculase II Fusion polymerase. Reaction was performed at 98° C. 2 mins, 98° C. 30 s, 60° C. 30 s, 72° C. 1 min for 10 cycles followed by 72° C. extension for 10 mins. Amplified library DNA was purified using 1.8× AMPure XP beads. Following purification, subsequent enrichments can be performed by repeating the above procedures or library DNA can be quantified and sequenced. The comparison results are shown in Table 7.

TABLE 7
Three sequential PEC improves detection of HBV-JS reads in optimized panel

| | Two Enrichments | | | | Three Enrichments | | | |
|---|---|---|---|---|---|---|---|---|
| | # | | | | # | | | |
| Reconstituted | HBV-JS | TERT-JS | CCDC57-JS | MVK-JS | HBV-JS | | | |
| PLC | Reads | (UMI) | (UMI) | (UMI) | Reads | TERT-JS | CCDC57-JS | MVK-JS |
| 1%-A | 121 | 3 | 4 | 7 | 268 | 2 | 6 | 2 |
| 1%-B | 160 | 0 | 19 | 9 | 266 | 1 | 18 | 5 |
| 1%-C | 144 | 0 | 18 | 8 | 123 | 0 | 28 | 13 |
| 0.5%-A | 89 | 1 | 3 | 0 | 349 | 1 | 5 | 1 |
| 0.5%-B | 104 | 0 | 0 | 0 | 269 | 0 | 13 | 2 |
| 0.5%-C | 45 | 0 | 6 | 30 | 374 | 0 | 8 | 3 |
| 0.1%-A | 22 | 0 | 4 | 0 | 308 | 0 | 4 | 0 |
| 0.1%-B | 23 | 0 | 12 | 5 | 287 | 0 | 1 | 2 |
| 0.1%-C | 13 | 0 | 3 | 2 | 348 | 0 | 0 | 0 |

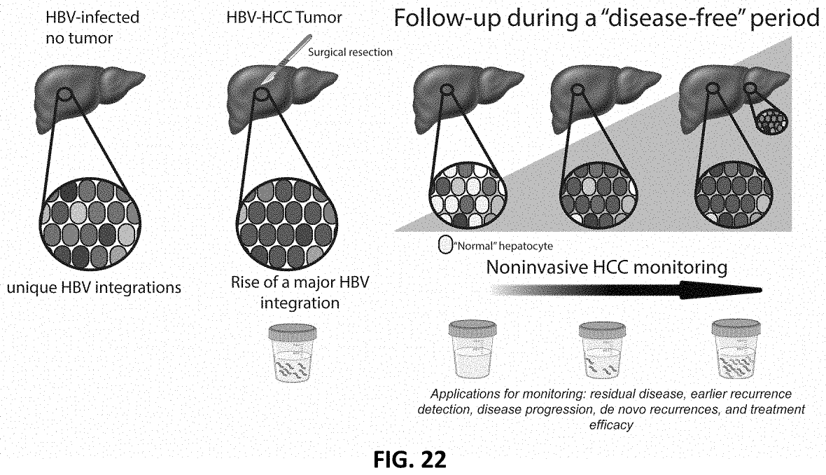

FIG. 22 illustrates the proposed applications for detection of major HBV-JS in urine of HBV-HCC patients for HCC disease management. Upon infection with HBV, integration of viral DNA into the host genome occurs in a number of liver cells. This will result in the generation of unique HBV-JS in each integrated hepatocyte (Note each color represents a hepatocyte with a unique set of HBV-JS, or molecular fingerprint). During HCC carcinogenesis where hepatocytes undergo clonal expansions, unique HBV-JS become clonally expanded (major junctions) in the tumor nodule and are detectable in urine prior to surgical resection. Frequent monitoring in urine during follow-up can serve as noninvasive way to monitor patients for residual disease, earlier recurrence, disease progression, de novo recurrence, and therapeutic efficacy for precision medicine.

### Example 2: Detection of Recurrent HBV Integration Targeted Genes in Urine Identifies Potential Drivers of Hepatocellular Carcinoma

Chronic hepatitis B virus (HBV) infection is a major etiology of hepatocellular carcinoma (HCC), associated with over 50% of cases worldwide and up to 70-80% of cases in HBV-endemic areas. High mortality of HCC is mainly due to late detection and limited treatment options. HCC surveillance programs have been implemented to screen HBV-infected individuals, to facilitate earlier detection of HCC. Unfortunately, most cases of HBV-related HCC (HBV-HCC) remain undetected until late stages resulting in poor prognosis, due to lack of a sensitive and convenient screening method. In the past years, over 100 clinical trials for HCC therapy failed, Sorafenib, with a limited efficacy, remains the only available chemotherapy after its approval 9 years ago. Identification of HCC drivers has been suggested to be important for drug development and patient selection in clinical trial design due to high heterogeneity of the diseases (REF).

During the course of infection, HBV can integrate into the host chromosome, and this integrated viral DNA was detected in more than 85% of HBV-HCC. Although it is known that viral breakpoints predominately occur in the DR1-2 region of the HBV genome, the integration sites in the host DNA have been observed to vary. Thus, each HBV integration event generates a unique HBV-host integration site, which creates a specific fingerprint of each infected hepatocyte. During the tumorigenesis, uncontrolled clonal expansion can amplify this molecular signature becomes a major, most abundant, over other host junctions found in other noncancerous infected hepatocytes. Thus, the merging of this uncontrolled, clonally expanded major HBV-host junction can be a biomarker for carcinogenesis, and can be a biomarker for early detection of HCC if this major HBV-host junction can be detected in periphery.

In order to test the feasibility to detect HBV-host junctions in circulation, urine was resorted since it is limited, if any of virions thus facilitating detection of integrated HBV DNA. It has been shown that urine contains DNA from circulation that can be used for cancer detection if a tumor is present. Although HBV DNA has been detected in urine, it has not been entirely clear if HBV DNA detected in urine was derived from fragmented integrated DNA from infected liver. In this proof-of-concept study, a method is developed to prepare a DNA library for NGS enriched for HBV integration. Using this approach, identical, major HBV integration sites from matched HCC tissue and urine are detected, providing evidence that clonally expanded, integrated HBV DNA derived from the infected liver is present in the urine. Combining this data with other reports of HBV integration, it was found the recurrently targeted genes are mostly associated with carcinogenesis suggesting potential approach for HBV-HCC driver identification. In particular, the TERT gene seems to be highly targeted within a narrow range of the promoter region. Together, these results not only suggest the utility of urine as a body fluid to study HBV integration sites in circulation, but also describe a noninvasive means for potential HCC screening and genetic characterization.

### Experimental Procedures

Study subjects: the HCC tissue and urine samples used were obtained with written informed consent from patients at the National Cheng-Kung University Medical Center, Taiwan, in accordance with the guidelines of the Institutional Review Board. Detailed sample information is provided in Table 8.

TABLE 8
Clinical characteristics of HCC patients.

| Patient | Age | Gender | Cirrhosis | Tumor | Tumor size |
|---|---|---|---|---|---|
| ID | (years) | (M/F) | (+/-) | grade* | (cm) |
| 1 | 71 | M | + | G1 | 3.5 |
| 2 | 68 | M | − | NA | NA |
| 3 | 44 | F | − | G3 | 3.5 |
| 4 | 43 | M | − | G2 | 3.0 |
| 5 | 68 | M | − | G2 | 6.5 |
| 6 | 58 | M | − | G2 | 15.0 |
| 7 | 57 | M | − | G2 | 4.0 |
| 8 | 41 | M | + | G2 | 2.0 |
| 9 | 49 | M | + | G2 | 3.4 |
| 10 | 61 | M | − | G3 | 2.3 |
| 11 | 75 | F | − | G2 | 3.0 |
| 12 | 63 | F | − | G2 | 4.0 |
| 13 | 39 | F | − | G2 | 10.0 |
| 14 | 59 | F | + | G2 | 4.0 |
| 15 | 47 | F | + | G2 | 1.5 |
| 16 | 63 | F | + | NA | NA |
| 17 | 29 | M | + | G2 | 7.0 |
| 18 | 33 | F | + | G1 | 2.5 |
| 19 | 61 | M | + | G3 | 7.0 |
| 20 | 57 | M | + | G1 | 3.0 |
| 21 | 73 | M | + | G2 | 11.0 |
| 22 | 42 | M | + | G2-G3 | 6.0 |
| 23 | 75 | M | − | G2 | 1.9 |
| | 55.5 ± 13.6 | 15/8 | 11/12 | | |
| | (Avg. ± SD) | (M/F) | (−/+) | | |

*denotes HCC tumors were staged using the tumor-node metastasis (TNM) staging system;
NA, Not applicable

DNA isolation, urine collection, and low molecular weight (LMW) urine DNA fractionation: Tissue DNA was isolated using the Qiagen DNeasy Tissue kit (Valencia, Calif.) according to the manufacturer's instructions. Urine samples were collected and total urine DNA was isolated as previously described (Su Y H et al. 2004). Cell-free DNA (<1 kb) was obtained from total urine DNA using carboxylated magnetic beads, as previously developed (Su Y H et al. 2008).

Preparation of HBV DR1-2 enriched library DNA for NGS: Tissue DNA was fragmented by sonication and subjected to Next-Generation Sequencing (NGS) library DNA preparation as described by Ding et al. 2012. This involved minor modifications, including 10 cycles of library DNA amplification using Herculase II Fusion polymerase (Agilent Technologies, Santa Clara, Calif.). To enrich for DNA that contains HBV DR1-2 sequences, a multiplex biotin HBV primer extension reaction was performed using amplified library DNA in a reaction containing 1× Herculase II Buffer, 250 μM dNTP, and 20 pmol of biotinylated HBV primers. The primer-extended DNA was collected, as described by Gnirke et al. 2009, subjected to three individual nested HBV DR1-2 PCR enrichment reactions, and followed by an indexing PCR. Each indexed library was quantified and pooled accordingly for one NGS. NGS was performed to generate 150 bp paired-end reads on the Illumina MiSeq platform (Penn State Hershey Genomics Sciences Facility at Penn State College of Medicine, Hershey, Pa.).

Identification and characterization of HBV-JS sequences: NGS data was analyzed using JBS ChimericSeq software (http://www.jbs-science.com/ChimericSeq.php, Jongeneel et al. manuscript submitted) to identify integration sites and major integration sites. For all the major integration sites identified, the software provided the annotation of breakpoints for both the HBV genome and human genome, human genes within 100 kb of the breakpoints, the number of overlapping viral and human nucleotides at the junction site and the Tm of the overlapping sequences.

Short amplicon PCR assays: Short amplicon junction PCR was performed using Hotstart Plus Taq Polymerase (Qiagen, Valencia, Calif.), junction primers, and the LMW urine DNA templates. Junction PCR products were visualized on a 2.2% FlashGel DNA Cassette (Lonza Group, Basel, Switzerland) and subsequently subjected to either a nested PCR reaction using a set of inner primers, or a restriction endonuclease (RE) digestion using RE obtained from New England Biolabs (Ipswich, Mass.), per the manufacturer's specifications to further compare the PCR products derived between tissue and urine.

Results:

Development of an NGS Library Enrichment Method for HBV Integrations:

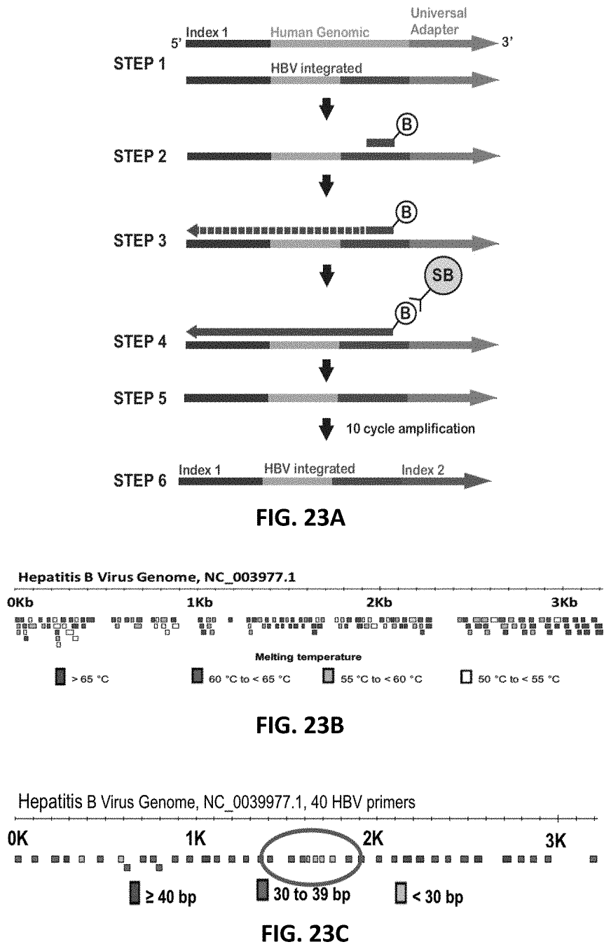

To directly enrich for HBV integrated DNA, a primer extension capture (PEC) approach was adopted to the HBV DNA libraries. In short, this technique uses 5′-biotinylated oligonucleotide primers to capture targeted regions, and then uses a DNA polymerase to extend the primers (FIG. 23A). This approach combines selectivity of the primer with high affinity of the extension, resulting in high recovery and enrichment of target sequences from an adapter-ligated DNA library. In designing the biotinylated primers for HBV capture, regions of sequence similarity between the human genome and the 3.2 Kb viral HBV genome were mapped. Through extensive BLAST analysis, 142 microhomologous regions were identified, depicted as shaded blue boxes in FIG. 23B. In order to avoid these regions, a set of short primers with minimal overlap with human homologous regions containing high melting temperatures (FIG. 23C) were constructed. These primers were further targeted to the DR1 and DR2 regions of HBV, since these are known integration hotspots with nearly 80% of breakpoints being reported in these regions. This was to more effectively identify the junction sites of HBV integrated DNA.

Identification of Major HBV Integration Sites from HCC Tumor Tissue Using PEC of HBV DR1-2:

In order to test whether the PEC approach was effective at enriching HBV integrated DNA from a biological sample, this technique was applied to an adapter-ligated tissue DNA library of 23 patients with chronic HBV infection and hepatocellular carcinoma (HCC). With the assumption that sampled tumors contain HBV integrated DNA at 1:1 ratio with human genomic DNA, A 10E4-fold enrichment would be necessary to obtain 1% HBV reads out of total reads. Through improving the specificity by PEC, it is able to obtain an average of 3.5% HBV reads of total NGS reads (data not shown).

Tumors are clonally expanded and most HBV-HCC tumors contain integrated HBV DNA (Ref), thus should contain at least one major, clonally expanded, HBV integration junction. In this study a major integration junction is defined as a distinctively identified sequence supported by at least 10% of the total HBV junction reads (minimum of 3 reads) within each DNA tissue sample. Reads containing HBV junctions were efficiently identified using the recently developed software program, ChimericSeq as described in Methods. The major HBV integration junctions identified in the NGS data by ChimericSeq are summarized in Table 9.

TABLE 9
Characterization of major HBV integration sites identified in HBV-HCC tissue.

| | | | HBV-host junction | | | |
|---|---|---|---|---|---|---|
| | | | breakpoint nucleotide (nt.) | | | |
| | | | position | | | Sanger |
| Patient | | # of | | | overlap | sequencing |
| ID | HBV integration site sequences | SR′/TR | HBV | Human | nt./Tm(° C.) | confirmed |
| 1 | cgaccttgaggcatacttcaaagactgtttgtttaaagactgggaggagtt | 20/20 | 1773 | Chr5: | 3/12 | + |
| | gggggaggagattaggaggctgtaggcataaaGGAAGGGGAG | (100%) | | 1295082 | | |
| | GGGCTGGGAGGGCCCGGAGGGGGCTGG (SEQ | | | | | |
| | ID NO: 178) | | | | | |
| 2 | gggggaggagataaggttaaaggtctttgtactaggaggctgtaggcat | 24/24 | 1801 | Chr5: | 1/10 | + |
| | aaattggtctgCCCAGCCCCCTCCGGGCCCTCCCAGC | (100%) | | 1295123 | | |
| | CCCTCCCCTTCCTTTCCGCGGCC (SEQ ID NO: | | | | | |
| | 179) | | | | | |
| 3 | gaggagattaggctaaaggtctttgtactaggaggctgtaggcataaatt | 76/77 | 1820 | Chr19: | 3/14 | + |
| | ggtctgttcaccagcaccatgcaacGGAGCTCATAACCTGAT | (98.7%) | | 29812873 | | |
| | CAGCTTTCTCTTCTTCTCTCTGTTTTTGTCTTGTTT | | | | | |
| | GGTGTGTTTCCTTGGGGTCATGG (SEQ ID NO: | | | | | |
| | 180) | | | | | |
| 4 | gggggaggagataaggttaaaggtctttgtactaggaggctgtaggcat | 68/123 | 1827 | Chr8: | 1/10 | + |
| | aaattggtctgttcaccagcaccatgcaactttttccTTTTCTATATC | (55.2%) | | 64147161 | | |
| | AATTGTTGATACTCCAATAATATTAATTGCTAAG | | | | | |
| | (SEQ ID NO: 181) | | | | | |
| | gggggaggagataaggttaaaggtctttgtactaggaggctgtaggcat | 30/123 | 1795 | Chr.9: | 0/0 | NA |
| | aaattTTATCTTCATATAAAATCTAGACGGAAGCAT | (24.3%) | | 45073810 | | |
| | (SEQ ID NO: 182) | | | | | |
| 5 | actaggaggctgtaggcataaattggtctgttcaccagcaccatgcaact | 28/30 | 1801 | Chr20: | 5/18 | + |
| | ttttcTTATGAATGTTTTCTATATTTCAAAGCCCTGCT | (93.3%) | | 53437062 | | |
| | CAAACACCACCTCCTCCAGAAAGGCTCCTGGTAT | | | | | |
| | CCTCTTTCTTTTCTAACCTAGAAAAGA (SEQ ID | | | | | |
| | NO: 183) | | | | | |
| 6 | gcctaatcatgtcatgttcatgtcctactgttcaagcctccaagctgtgcctt | 34/70 | 1901 | Chr19: | 1/10 | + |
| | gggtggctttggggcatgCGGGTGCCCGGGTCGCGGGT | (48.5%) | | 29812390 | | |
| | GACAGGCCACCCCGCCATCGGCCATCTTCCTGG | | | | | |
| | CTCGCCCGGCCGCCCGCGCGCA (SEQ ID NO: | | | | | |
| | 184) | | | | | |
| | gactctcagcaatgtcaacgaccgaccttgaggcatacttcaaagactg | 28/70 | 1765 | Chr.6: | 0/0 | NA |
| | tttgtttaaggactgggaggagttgggggaggagattaggttaaagaTT | (40%) | | 17125139 | | |
| | ACCATGTTGCCCAGGCTGGTCTTGAACACCTGGC | | | | | |
| | CTCAAGGGACGCTCCCAGC (SEQ ID NO: 185) | | | | | |
| 7 | cacgtcgcatggagaccaccgtgaacgcccaccaagtcttgcccaag | 13/21 | 1712 | Chr.10: | 9/28 | + |
| | gtcttacataagcggactcttggactcccagcaatgtcaacgaccgacct | (61.9%) | | 31192695 | | |
| | tgaggcgtacttcaaaACCCAGACCCAGCTCAGGCATC | | | | | |
| | ACCACCTCCAGGCAGC (SEQ ID NO: 186) | | | | | |
| 8 | tggactttcagcaatgtcaatgaccgaccttgaggcatacttcaaagact | 27/27 | 1756 | Chr.11: | 6/24 | + |
| | gtgtgtttactgagtgggaggagttgggggagggactagCTCATTA | (100%) | | 92048629 | | |
| | ATCATTGTGTCAAACCTGGCACCGTGCCTGAAAC | | | | | |
| | ACAGTAGCCTCTCAATAAATA (SEQ ID NO: 187) | | | | | |
| | ttgcacaacaggactcttggacTTACACCAGTGGTTTGCC | NA | 1672 | Chr.22: | 13/46 | + |
| | GGGGAATCTTGAGCCTTTGGCCACAGACTGAAG | | | 34131795 | | |
| | GCTGCACTGTCAGCTTCCCTACTTTTGAGGCTTT | | | | | |
| | CG (SEQ ID NO: 188) | | | | | |
| 9 | actaggaggctgtaggcataaattggtctgttcaccagcaccatgcaact | 4/4 | 1825 | Chr.16: | 1/8 | + |
| | ttttTCCGAACCTGTGTACTAAACTGCCTGGGGGCA | (100%) | | 29467674 | | |
| | GCTCTCATCACTGCTGTAGAACAAAGTCCCACAT | | | | | |
| | AGAGCCAATGGCCAAGAACCAGTTAATAAAA | | | | | |
| | (SEQ ID NO: 189) | | | | | |
| 10 | gagtgggaggagttgggggaggagattaggttaaaggtctttgtactag | 85/206 | 1783 | Chr.5: | 3/16 | + |
| | gaggctgCATGGCCGGAAGTCTTACATGTCTTGGG | (41.2%) | | 1292170 | | |
| | AGTTTGTGGGGAGGGGGTGAAATCGGGACTTCT | | | | | |
| | TCTAGCTGCCACGG (SEQ ID NO: 190) | | | | | |
| 11 | gggggaggagataaggttaaaggtctttgtactagtaggctgtaggcat | 26/46 | 1796 | Chr.14: | 0/0 | + |
| | aaattgCCTACAGCAATGTATAGATTTTAAATAAATG | (56.5%) | | 67004392 | | |
| | CTTGCTGACTTACTATGACCTACTGGTAG (SEQ ID | | | | | |
| | NO: 191) | | | | | |
| 12 | gactcttggactcccagcaatgtcaacgaccgaccttgaggcctacttca | 50/71 | 1780 | Chr.5: | 8/32 | + |
| | aagactgtgtgtttaaggactgggaggagctgggggaggagattaggtt | (70.4%) | | 165559760 | | |
| | aatgatctttgtactaggaggAACATGCCCAAGAAATTGGC | | | | | |
| | GACATACCAGC (SEQ ID NO: 192) | | | | | |
| 13 | tcttgcataagaggactcttggactttcggcaatgtcaacgaccgaccttg | 5/6 | 1726 | Chr.14: | 2/10 | + |
| | aggcatacttcaaagactgtgtgtttaaCTCATCTGTCCAAACC | (83.3%) | | 103176895 | | |
| | CAAAGAATGGACTCAGAGACCCAGAGAACAACGA | | | | | |
| | AAGTGACGGTTTGTTCTT (SEQ ID NO: 193) | | | | | |
| | ttgcacaacaggactcttggactctcagcaatgtcaacgaccgaccttga | NA | 1742 | Chr.19: | 9/9 | + |
| | ggcatacttcaaagactgtttgtttaaagactgggaggagttgCATCT | | | 53667608 | | |
| | AACTCAGGTTTTCAACTAGTCTTACCATTGAAAGA | | | | | |
| | ACTATTGTGGCAAAGACGGAATG (SEQ ID NO: | | | | | |
| | 194) | | | | | |
| 14 | aaagactgggaggagttgggggaggagattaggttaaaggtctttgtac | 18/38 | 1803 | Chr.7: | 13/38 | - |
| | taggaggctgtaggcataaattggtctgttTGAAGTTGTCCAG | (47.3%) | | 4338599 | | |
| | AAACTGACCTTTGAATATCCGGATGCACGAGATT | | | | | |
| | CCCTGAAAGGGGAACAATAAATGT (SEQ ID NO: | | | | | |
| | 195) | | | | | |
| | caaggtcttacataagcggactcttggactctcagcaatgtcaacgacc | 13/38 | 1713 | Chr.X: | 0/0 | - |
| | gaccttgaggcgtacttcaaaggTGTTACAGGTAGTTAGAC | (34.2%) | | 35786804 | | |
| | AGGCATGAGCAGGGCAGGAGAGAACGCTCCCCT | | | | | |
| | GACTCACCAGGAATGTCAGGCAATCATTG (SEQ | | | | | |
| | ID NO: 196) | | | | | |
| 15 | tgggaggagttgggggaggagattaggttaatgatctttgtactaggagg | 9/9 | 1826 | Chr.4: | 9/22 | - |
| | ctgtaggcataaattggtgtgttcacctgcaccatgcaactttttcTGGG | (100%) | | 141291543 | | |
| | GATGGGGATGTGGCAGTTGTGGACTGAAGTTGTA | | | | | |
| | CTGAGTGGTG (SEQ ID NO: 197) | | | | | |
| 16 | ttaggttaatgatctttgtactaggaggctgtaggcataaattggtctgAC | 22/23 | 1801 | Chr.5: | 1/10 | NA |
| | CCGCCCTTCTCTGCCCAGCACTTTTCTGCCCCCC | (95.6%) | | 1299125 | | |
| | TCCCTCTGGAACACAGAGTGGCAGTTTCCACAAG | | | | | |
| | CACTAAGCATCCTCTTCCCAAAAGACCCAGC | | | | | |
| | (SEQ ID NO: 198) | | | | | |
| 17 | aacagtctttgaagtacgcctcaaggtcggtcgttgacattgctgagagtc | 7/8 | 1623 | Chr.9: | 0/0 | NA |
| | caagagtccgcttatgtaagaccttgggcaagacctggtgggcgttTG | (88%) | | 16709453 | | |
| | GTGGCATTGCAAGTGTACTGTTTAA (SEQ ID NO: | | | | | |
| | 199) | | | | | |
| 18 | cacaacaggactcttggactctcagcaatgtcaacgaccgaccttgagg | 30/103 | 1814 | Chr.5: | 13/40 | NA |
| | catacttcaaagactgtgtgtttaaagactgggaggagttgggggagga | (29.1%) | | 1284093 | | |
| | gattaggttaaaggtctttgtactaggaggctgtaggcataaattggtctg | | | | | |
| | gacctgcatcatCCGGACTCCATAC (SEQ ID NO: 200) | | | | | |
| | tgcacaacaggactcttggactctcagcaatgtcaacgaccgaccttga | 30/103 | 1802 | Chr.19: | 1/4 | NA |
| | ggcatacttcaaagactgtgtgtttaaagactgggaggagttgggggag | (29.1%) | | 29812598 | | |
| | gagattaggttaaaggtctttgtactaggaggctgtaggcataaattggtct | | | | | |
| | gcCCGCGGCCCGGGCACTCACCGCTCCCTGCGC | | | | | |
| | TCCCTCGGCATGATGGGGCTGCTCCGG (SEQ ID | | | | | |
| | NO: 201) | | | | | |
| | tgcacaacaggactcttggactctcagcaatgtcaacgaccgaccttga | 17/103 | 1765 | Chr.4: | 9/24 | NA |
| | ggcatacttcaaagactgtgtgtttaaagactgggaggagttgggggag | (16.5%) | | 116834523 | | |
| | gagattaggttaaaggtGTCTGGTATTATTTCTGGGTTCT | | | | | |
| | CTATTCTGTTCC (SEQ ID NO: 202) | | | | | |
| | tttaaagactgtgaggagttgggggaggagattaggttaacggtctttgtg | 10/103 | 1781 | Chr.14: | 5/18 | NA |
| | ctgtggaggGAAGACTAAGTAGAGACGCGGATGTTT | (9.7%) | | 32527123 | | |
| | ATGGCAGTGAAACTGTTC (SEQ ID NO: 203) | | | | | |
| 19 | cgaccttgaggcatacttcaaagactgtttCAAGAAACTGAGT | 192/255 | 1720 | Chr.9: | 12/32 | NA |
| | GAGTAGGCTCTGGAAATTGGAAGTGATCTTAGTA | (75.2%) | | 22215960 | | |
| | TTTAAGTTCAGTCACTCAACTACAATCTCTGAAAC | | | | | |
| | (SEQ ID NO: 204) | | | | | |
| | cgaccttgaggcatacttcaaagactgtttTACCAGACACTCAC | 14/255 | 1722 | Chr.X: | 7/18 | NA |
| | ATGGCTTCCTCGCTGTCTTCCTGTGGTGGCACAC | (5.4%) | | 130398440 | | |
| | GCCTGTAGTCCCAGCTACTTGGGAGGCTGAGGC | | | | | |
| | AGGAGAATTGCTTGAACC (SEQ ID NO: 205) | | | | | |
| 20 | gggggaggagataaggttaaaggtctttgtactaggaggctgtaggcat | 20/20 | 1826 | Chr.12: | 0/0 | NA |
| | aaattggtctgttcaccagcaccatgcaactttttcTGGTGAAAAGC | (100%) | | 74945009 | | |
| | TAAACACAGGAGATATTTTTAAGCTTCACTCATAC | | | | | |
| | AGAAAATACA (SEQ ID NO: 206) | | | | | |
| 21 | gggggaggagataaggttaaaggtcttgtttgtactaggaggctgtagg | 6/6 | 1800 | Chr.10: | 2/8 | NA |
| | cataaattggCAGGACCCAGGGGAGCAGCCAGCACT | (100%) | | 124355242 | | |
| | GCGCATGCTGGGAGTGTTCAATAAATACAGGCTG | | | | | |
| | AATGAATGAATGAACTGATGCATCCAAAACTT | | | | | |
| | (SEQ ID NO: 207) | | | | | |
| 22 | cgaccttgaggcatacttcaaagactgtttgGAAAAAATGTAAA | 10/86 | 1723 | Chr.12: | 9/26 | NA |
| | CATATCAGCCCTGAGCAAGACAGCCAAACCAAAA | (11.6%) | | 40055103 | | |
| | CAACCACAGCGAGGGATTCTGATTCCTTTGACAG | | | | | |
| | ACTCTGTTTCT (SEQ ID NO: 208) | | | | | |
| | cgaccttgaggcatacttcaaagactgtttgCCTTTTCCCCTAA | 9/86 | 1731 | Chr.2: | 12/32 | NA |
| | TCCCCTTTCCCCACTGGTACAGGGTGGAGAGGT | (10.4%) | | 44149056 | | |
| | C (SEQ ID NO: 209) | | | | | |
| 23 | gactcccagcaatgtcaacgaccgaccttgaggcctacttcaaagactg | 7/25 | 1803 | Chr.1: | 0/0 | NA |
| | tgtgtttaaggactgggaggagctgggggaggagattaggttaaaggtc | (28%) | | 39524755 | | |
| | tttgtattaggaggctgtaggcataaattggtctgCGTCACCCTCC | | | | | |
| | AGAAGGA (SEQ ID NO: 210) | | | | | |
| | cacaacaggactcttggactcccagcaatgtcaacgaccgaccttgag | 6/25 | 1811 | Chr.14: | 3/10 | NA |
| | gcctacttcaaagactgtgtgtttaaggacttggaggagctgggggagg | (24%) | | 95571897 | | |
| | agattaggttaaaggtctttgtattaggatgctgtaggcataaattggtctg | | | | | |
| | cCTTGACTAAAGCCCATGGGCCA (SEQ ID NO: | | | | | |
| | 211) | | | | | |

To confirm the junction sequences obtained from the NGS analysis, PCR primers were designed for the major HBV integration junctions of 15 patients and performed amplification from the corresponding tissue DNA for Sanger sequencing. The respective tissue NGS library DNA was used as a positive control (+) for the junction sequence identified by NGS and HepG2 cell line DNA as a negative control (−) for each DNA tissue sample. Encouragingly, it was able to generate PCR products for 13 out of 15 of the tissue DNA samples tested. Only 2 of the 15 samples (patients 7 and 8) were unable to generate a PCR product using custom primers (data not shown). Further Sanger sequencing of each PCR product revealed matching HBV integrated sequences to their corresponding NGS-identified integration sequence, thus confirming the 13 samples. In total, it was able to validate 87% (13/15) of the major NGS identified HBV integration sites.

Detection of Tissue Identified Major HBV Integration Sites in Matched Urine:

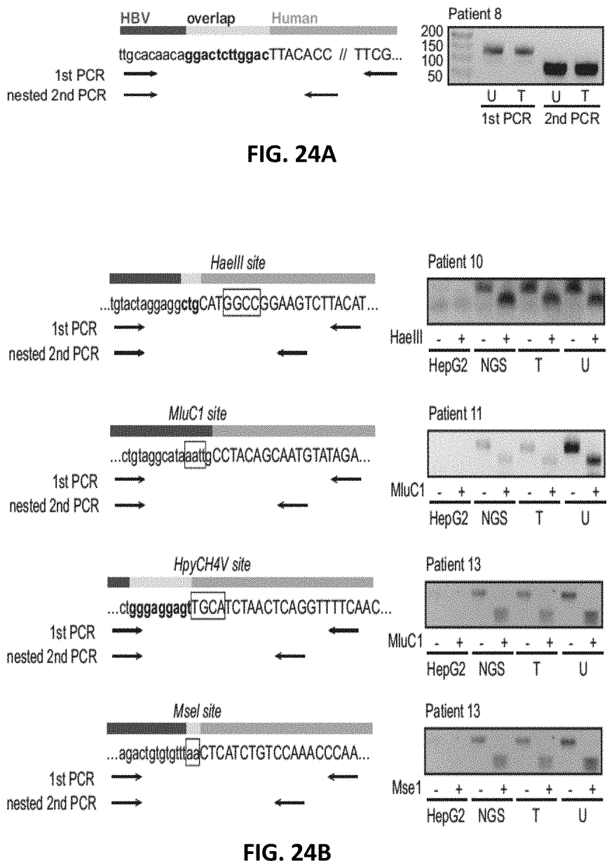

Next it is examined whether major HBV integration sites can be detected in the circulation. As previously demonstrated, urine contains circulation derived DNA. The use of urine over serum collection is also advantageous, as it does not contain high amounts HBV DNA from virions in the circulation. In order to test the feasibility of detecting HBV integration junction sequences in the urine, seven patients (ID 9, 10, 11, 12, 13, 14, and 15) that have major HBV integration junctions identified by NGS study from this study were selected for this experiment based on the availability of matched urine DNA. For each major HBV integration site, primers were custom designed to amplify short products of less than 60 bp (illustrated in FIG. 24A), which consisted of one primer targeting the HBV sequence and the other primer targeting the human sequence near the junction. For patient 10, a nested PCR approach was used to confirm the integration site since the length of the PCR product was sufficient for the nested PCR primer design (FIG. 24A). In all other cases, a PCR approach was carried out where amplicons were digested with a specific restriction endonuclease to validate the PCR product sequences generated from tissue DNA is similar to that of urine DNA for patients 10, 11, and 13 (FIG. 24B).

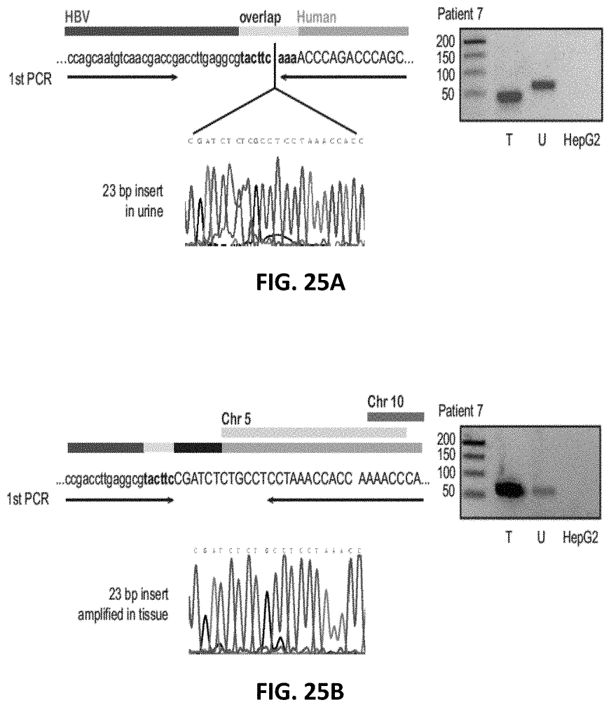

Interestingly, for patient 7, the PCR product generated from urine DNA was larger than the one obtained from the tissue by PCR amplification (FIG. 25A). To determine whether these two junction DNA species were related, the PCR product derived from urine DNA was analyzed with Sanger sequencing. A 23 nucleotide (nt) insert was identified, joined between HBV DNA and chromosome (Chr) 10. By an NCBI Blast analysis, a 21 nt stretch of the chimeric sequences identified in urine was found to have 100% homology to Chr 5. Next it was determined whether this urine-derived 23 nt insert junction sequence could be identified in the corresponding tissue DNA. A primer is designed across the chimeric sequences between Chr 5 and Chr 10, as illustrated in FIG. 25A, to amplify this urine-identified 23-nt inserted HBV-JS in the corresponding tissue DNA. As expected, this urine derived HBV-JS was detected by PCR in the tissue DNA and the sequences were confirmed by Sanger sequencing, as shown in FIG. 25B. Together with the confirmed samples in FIGS. 24A and 24B, it is able to detect and verify six of nine HBV integration sites identified from HBV-HCC tissues in the matched urine samples.

Major HBV Integrations in HCC Recurrently Target TERT and CCNE1:

In HBV-infected individuals, integration into the host genome is thought to be random, having the potential to become oncogenic by insertional mutagenesis. The HBV DR1-2 sequences contain enhancer elements that may up-regulate host genes within a proximity of 100 kb, independently of position and orientation. With the identified locations of major HBV integration sites in HCC patients, host genes within 100 kb of these major sites were searched. ChimericSeq is used to identify the genes and positions of each breakpoint in both HBV and human genomes from the NGS data from tissue DNA. Out of the 34 major integration sites that were identified in 23 patients, 4 were not in a 100 kb proximity of a gene. Among these genes, TERT and CCNE1 were targeted in more than 1 patient; TERT was targeted in 5 of the 23 patients from this study, and CCNE1 was targeted in 3. Interestingly, both genes were found to be associated with carcinogenesis. Indeed, TERT is a suggested gatekeeper of hepatocarcinogenesis as the promoter region is frequently mutated in certain cancers. It thus was wondered whether identification of recurrent integration targeted genes could be a potential approach to identify drivers involved in hepatocarcinogenesis.

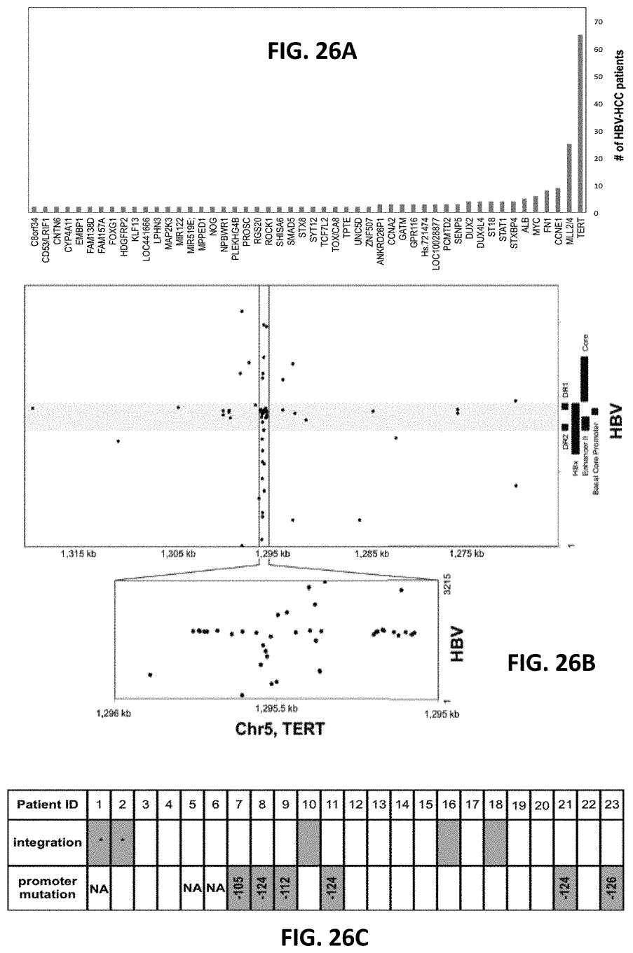

To explore this hypothesis, a meta-analysis of data reported from 15 studies, 446 patients, and 1554 HBV integrations was compiled. ChimericSeq was used again on this data set to identify genes within 100 kb of the integration site. From the 51 genes that were identified in at least 2 HCC patients, 12 were from at least two separate studies, defined as HBV integration recurrently targeted genes in HCC (FIG. 26A). Most strikingly, 10 of the 12 recurrent targeted genes have reported association with cancer. This aligns with the identification of recurrently mutated driver genes in HCC carcinogenesis, and suggests that identification of recurrently integrated genes could identify drivers.

In alignment with this study of 23 HCC tumor tissues, TERT and CCNE1 were among the most common recurrent integration sites. Because of the presence of the most data for integrations near TERT, these 67 integration sites were compiled for further study. First, the location of the TERT integration breakpoints in the host genome was mapped against their locations in the HBV genome (FIG. 26B). Interestingly, the majority of HBV integrations targeted within a 1 kb stretch of the TERT promoter, of which a majority of breakpoints from the HBV genome are with the DR1-2 region. Even more noteworthy is that none of these integrations are identical, despite the high prevalence of integrations in a narrow region of the TERT promoter. This supports the view that HBV integrations in HCC are random in a sense that they do not occur in a sequence-specific manner.

Promoter mutations and upstream rearrangements of the TERT gene including HBV integration, are known factors that drive carcinogenesis. It was of interest to investigate the distribution of these two events in the same tumor. The TERT promoter region of 20 of 23 tissue samples was successfully sequenced from the study, and identified 5 mutations of which 3 are of the major TERT hotspot mutation (˜124) (FIG. 26C). Interestingly, the HCC tumors with TERT integration and promoter mutations were mutually exclusive events in this study.

Discussion:

This is the first study demonstrating that liver-derived HBV integration junction sequences can be detected in urine. This was enabled through the identification of the major integration site(s) in HCC tissue, followed by validation using tailored primers for these major sites from urine. The novel sequence created by HBV integration was taken advantage of, using it as a unique marker to trace for the HBV-integrated DNA that was released into circulation, and demonstrated the detection of identical integration sequences between the tumor tissues and corresponding urine samples. Detection of such unique sequences in the urine provides unambiguous evidence that HBV integrated DNA from the liver is released into circulation, and is filtered into urine as fragmented, cell-free DNA.

Two important features of HBV integration are foundations of this proof-of-concept study. First is the appearance of over-represented or major HBV integration sites in HCC due to uncontrolled clonal expansion, as demonstrated in earlier studies. While proliferation of infected hepatocytes can occur in non-HCC liver disease, mostly within 10⁵ cells, clonal expansion observed in HCC tumors is uncontrolled. This results in expansion of ˜10⁹ cells (1-3 cm tumor size), and results in preferentially abundant HBV integrated sequences in the infected liver or in the HCC nodule. This is shown in the supporting reads, which describes as the major HBV integrations in the NGS study (Table 9). Because of their high abundance, it was reasoned that these major HBV integration sites in the infected liver would most likely to be predominantly detected in urine. As predicted, major HBV integrations sites were detected in matching urine samples in six of nine HCC patients tested.

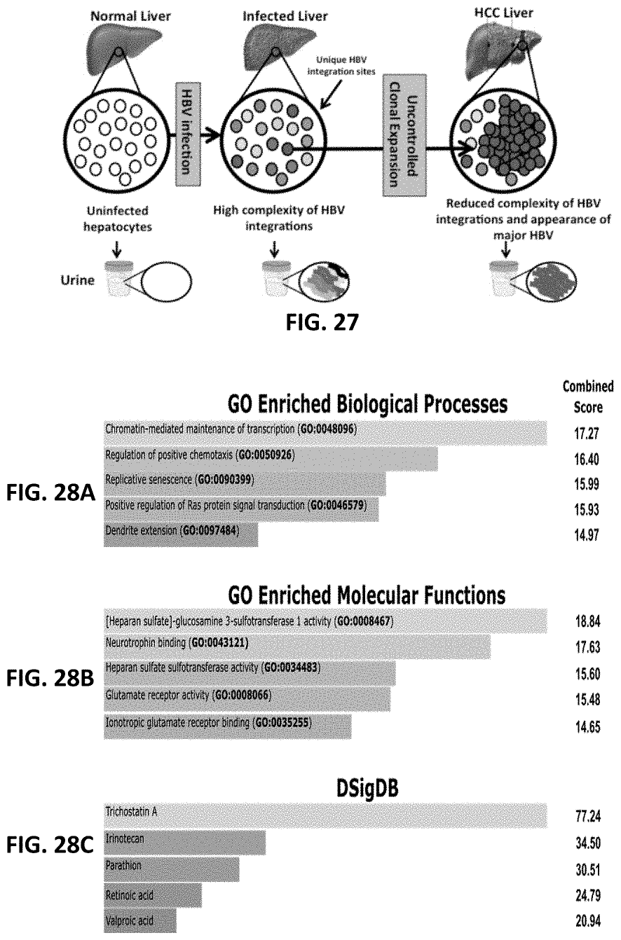

Second, the HBV integration events are random, and HCC-derived integration sites have previously been used as a cellular signature of the clonality of HBV-HCC tumors. Among over a thousand HBV integration sites identified in recent NGS-based studies, the most frequently reported recurrent integration targeted gene is TERT. Strikingly, with over 60 HBV-TERT junction sequences reported, no two are identical at both viral and host breakpoints. This further supports the hypothesis that HBV integration sites created by integration could serve as a molecular signature of the infected hepatocyte. Therefore, detection of an emerging, predominant integration site in the urine could be a potential biomarker for an early clonal expansion or HCC in a chronic HBV infected individual, as illustrated in FIG. 27.

The mechanistic links between HBV integration and hepatocarcinogenesis have been suggested to include activation of oncogenic genes and induction of chromosomal instability. By analyzing 34 major integration sites from 23 HBV-HCC patients, five were targeted in proximity of the TERT gene, and three within range of the CCNE1 gene, both commonly recognized oncogenes. Three additional integration sites at TSHZ2, GPHN, and miR512-1 have also been reported to be associated with carcinogenesis. The integration site identified from patient #7 showed chromosomal rearrangement, a common event in cancer. This high frequency of integration in oncogenic genes and the evidence of chromosomal instability detected in this study led people to study and compare other reports. Therefore, a meta-analysis of data reported from 15 studies, 446 patients, and 1554 HBV integrations was carried out. In line with this study, it was found that TERT and CCNE1 are among the most frequently reported targeted genes by HBV. Interestingly, it was observed that 10 other genes were targeted by separate studies from different groups, and most had previously reported association with cancer while other two functions are unknown. This indicated that while HBV integration may be random, disruption of particular regions might have more of an impact on development of HCC. Since TERT was by far the most commonly targeted gene, both the human and HBV genomic locations of each integration site were mapped. Strikingly, it was found that HBV integration is frequently observed in a narrow region of the TERT promoter, despite every integration site being unique. Since TERT promoter mutations are recognized drivers of carcinogenesis and TERT promoter integrations are mutually exclusive with these mutations, it is suggested that HBV integrations have the potential to act as drivers of carcinogenesis. Of note, the cohort in this small study was mostly of HBV-HCC patients that were predominantly non-cirrhotic (77%). This could imply that HBV integration plays a more direct role in HCC carcinogenesis in non-cirrhotic patients.

In moving forward, a more thorough analysis of HBV integration sites is needed to better assess the role of integration with carcinogenesis. While disruptions in TERT and CCNE1 appear to be well implicated in connection with development of HCC, there are likely several other important genes that are less frequently targeted. It was previously reported for. The detection of circulation derived DNA in the urine, and it thus believed that urine will be the best source to profile HBV integrations of the liver because unlike blood, urine contains limited (if any) infectious HBV particles. Even though HBV integrated DNA in the urine makes up only a very small fraction of total cfDNA, with advance in sensitivity of technology of detecting cfDNA, detection of major HBV integration sites in urine is plausible. As 85% of HBV-HCC samples were found to contain integrated HBV DNA, detection of the major HBV integration sites in urine could serve as a specific and sensitive marker for HCC screening of the chronic HBV infected population.

### Example 3: Landscape of Recurrently Targeted Genes by HBV Integration in Hepatocellular Carcinoma Patients: Potential Biomarkers for Disease Management

### 1. Introduction

Hepatocellular carcinoma (HCC) is the 2nd leading cause of cancer deaths worldwide [1-3], and suffers from poor prognosis in part due to lack of effective treatment options. The major etiology of this multifactorial disease is chronic hepatitis B virus (HBV) infection, which is associated with approximately 50% of HCC cases worldwide [4]. During the course of infection, HBV can integrate into the host genome. It has been believed that integration events mostly occur through non-homologous end joining (NHEJ) [5], as well as through micro-homologous recombination [6-9]. While HBV DNA integration into the host genome is considered rare, with an estimate of one integration event per ten thousand HBV-infected hepatocytes [10], the integrated viral DNA has been reported in more than 85% of HBV-related HCCs (HBV-HCC), suggesting a significant association of HBV integration in hepatocarcinogenesis. Mechanisms of HBV integration in HCC carcinogenesis could vary in patients and include insertional mutagenesis of HCC-associated genes, induction of chromosomal instability, and continuous expression of viral proteins [11,12]. Understanding the impact of integrated HBV DNA on carcinogenesis and potentially identifying HCC driver genes as personalized biomarkers could pave the way for precision disease management in HBV-HCC patients.

With the advent of next generation sequencing (NGS), thousands of HBV integration sites have been identified across the human genome. Over 15,000 HBV integration sites have been reported from PCR and NGS-based approaches from tumors [6,13-36]. While no known host sequence preference or specificity [5,37-41] was identified, integration can activate known HCC driver genes and has been reported in TERT, CCNE1, and MLL4 [42]. Integration in these genes has been reported in a recurrent manner (i.e. in more than one HCC patient) and have become known as recurrently targeted genes (RTGs). Interestingly, no RTG has been identified from non-HCC livers of chronically HBV-infected patients (n=90, 960 integration sites) [11, 27, 43, 44], suggesting its specificity for HBV-HCC. Similar to the approach of identifying BRAF V600E driver mutations by the identification of recurrent hotspot mutations, here we take advantage of the large amount of reported integration sites from literatures and our in-house study reported here to test the hypothesis that HCC drivers can be identified by characterizing RTGs.

In this study, we compared integrations sites identified in tumor and adjacent-to-tumor (adj-tumor) tissue and defined RTGs. By characterizing the top 10% most frequent RTGs, we demonstrate the potential of identifying HCC drivers for HCC precision medicine and drug development.

### 2. Results

2.1. Identification of RTGs in 22 HBV-HCC Tumors

The HBV DR1-2 region is a known integration hotspot. To identify HBV integration sites in a cost-effective manner, we applied an HBV DR1-2 enrichment NGS assay, as described in Materials and Methods, to enrich for HBV DNA in the DR1-2 region. NGS libraries prepared from archived DNA isolated from a cohort of 22 HBV-HCC formalin-fixed paraffin-embedded (FFPE) tissue specimens were used. NGS reads were analyzed using ChimericSeq [45]. We aimed to detect HBV junction sequences (HBV-JS) in 1-10 million NGS reads. Table 10 summarizes the NGS results and the major HBV-JS identified. Major HBV-JSs were defined as the most abundant HBV-JS in each tested sample that has at least 2 supporting reads and having more than 10% of total junction sequences. Assuming a 1:1 copy ratio of HBV to human genomic DNA, we obtained at least 1,000-fold enrichment resulting in an average of 1.0±0.3% on-target HBV reads (Table 10). Encouragingly, integrated HBV DNA was detected in 91% of HBV-HCC tumors from a 1-10 million NGS reads per sample (Table 10). Interestingly, of 27 major HBV-JS identified, seven junctions were found in frequently reported HCC driver genes (TERT and CCNE1) [46]. Junction-specific PCR primers were designed for 16 junctions with the most supporting reads and amplified in respective tissue DNA. PCR products for 14 of 16 tissue DNA samples were obtained and the junction sequences were confirmed by Sanger sequencing for an 88% validation rate (data not shown).

TABLE 10
Characterization of HBV-JSs identified in an in-house HBV-HCC
tissue cohort.

| | | On- | HBV-host junction | | |
|---|---|---|---|---|---|
| | | target | breakpoint nucleotide | | |
| Patient | Total NGS | HBV | (nt.) position | | |
| ID | Reads | Read % | HBV | Human | Gene |
| 1 | 6.12E+06 | 1.1% | 1773 | Chr5: 1295082 | TERT |
| 2 | 6.24E+06 | 1.1% | 1801 | Chr5: 1295123 | TERT |
| 3 | 6.35E+06 | 1.1% | 1801 | Chr5: 1299125 | TERT |
| 4 | 3.55E+06 | 0.8% | 1820 | Chr19: 29812873 | CCNE1 |
| 5 | 5.19E+06 | 1.2% | 1827 | Chr8: 64147161 | LOC102724623 |
| | | | 1795 | Chr9: 45073810 | Unknown |
| 6 | 7.24E+06 | 1.4% | 1801 | Chr20: 53437062 | LINE2 |
| 7 | 7.62E+06 | 1.3% | 1901 | Chr19: 29812390 | CCNE1 |
| | | | 1765 | Chr6: 17125139 | STMND1 |
| 8 | 2.11E+06 | 1.0% | 1712 | Chr10: 31192627 | LOC101929352 |
| 9 | 2.26E+06 | 1.0% | 1623 | Chr9: 16709453 | BNC2 |
| 10 | 1.17E+06 | 1.0% | 1756 | Chr11: 92048629 | LINE1 |
| 11 | 2.64E+06 | 1.1% | 1814 | Chr5: 1284093 | TERT |
| | | | 1802 | Chr19: 29812598 | CCNE1 |
| | | | 1765 | Chr4: 116834523 | HAVCR1P2 |
| | | | 1781 | Chr14: 32527123 | AKAP6 |
| 12 | 8.71E+06 | 1.3% | 1826 | Chr2: 74945009 | LOC105369842 |
| | | | 1722 | ChrX: 130398440 | RBMX2 |
| 13 | 5.78E+06 | 1.2% | 1800 | Chr10: 124355242 | OAT |
| 14 | 3.46E+06 | 0.9% | 1825 | Chr16: 29467674 | LOC388242 |
| 15 | 1.67E+06 | 1.2% | 1783 | Chr5: 1299170 | TERT |
| 16 | 3.73E+06 | 0.8% | 1796 | Chr14: 67004392 | GPHN |
| 17 | 4.42E+06 | 0.8% | 1772 | Chr19: 35403632 | LINC01531 |
| 18 | 1.22E+06 | 1.0% | | N.D. | |
| 19 | 5.04E+06 | 1.2% | 1803 | Chr1: 39524755 | Unknown |
| | | | 1811 | Chr14: 95571897 | LOC100506999 |
| 20 | 5.37E+06 | 1.2% | 1713 | ChrX: 35786804 | LTR Element |
| 21 | 9.00E+06 | 1.3% | | N.D. | |
| 22 | 3.84E+05 | 0.04% | 1727 | Chr14: 103176826 | LOC105370685 |
| Avg. ± | 4.36E+06 ± | 1.0% ± | | | |
| SD | 2.60E+06 | 0.3% | | | |

The nucleotide positions of the HBV (NC_003977.1) and human (GRCh38.p2) genome sequences at the HBV-human junction breakpoints. Within 150 kb of the HBV integration site breakpoint, the closest genes were identified by ChimericSeq software and listed as defined by NCBI's RefSeq gene database. Integration sites where no known gene was present within 150 kb are listed as “Unknown”.
N.D., no detectable HBV-host junctions;
Avg. ± SD, average ± standard deviation.

2.2 Overview of the Studies for RTG Identification

The studies included in RTG identification are summarized in Table 11, where 19 studies utilize NGS-based and 8 studies utilize PCR-based approaches for HBV integration identification. For each study, the sample size and the number and percentage of HCC tumor or adj-tumor tissue that had detectable integration sites are listed. Note, most of the studies did not examine the DNA from the adj-tumor. Together, we compiled a total of 15,749 integration sites: 8,491 from tumor tissues and 7,258 from the adj-tumor, from 1,023 HCC patients. We found 80% of tumor tissues (n=1,276) and 50% of adj-tumor tissues (n=760) contained detectable integration sites. Of the seven studies that enriched for the whole HBV genome, on average 81% (range 57%-100%) of the tumors examined were found to have integrated HBV DNA (n=7) [6,22-24, 26, 27]. In two studies, 65% [28] and 91% (our study) of tumors examined were positive for integrated HBV DNA.

TABLE 11
Summary of HBV integration junction studies included in this analysis.

| | | | | # of subjects with | | | | | | | | |
|---|---|---|---|---|---|---|---|---|---|---|---|---|
| | | | | integrated DNA* | | | | | | | Information | |
| | | | HCC | identified, | | | | # of junctions* | | | availability | |
| | | | patients | (% of total) | | | | identified in subjects | | | Junction | Clinical |
| | | Study | (n) | Tumor | | Adj. | | Tumor | Adj. | Total | sequence | variables |
| NGS- | WGS | [13] | 3 | 3 | (100%) | 3 | (100%) | 15 | 33 | 48 | Yes | Yes |
| based | | [14, 15] | 91¹ | 64 | (45%) | NA | | 223 | NA | 223 | Yes | Yes |
| | | [16, 17] | 81 | 76 | (94%) | 27 | (33%) | 344 | 55 | 399 | Yes | Yes |
| | | [18] | 2 | 2 | (100%) | NA | | 5 | NA | 5 | Yes | Yes |
| | | [19] | 5^{1,2} | 5 | (100%) | 4 | (33%) | 92 | 54 | 146 | Yes | — |
| | | [20] | 5 | 5 | (1005) | NA | | 21 | NA | 21 | Yes | Yes |
| | | [21] | 3 | 2 | (67%) | NA | | 11 | NA | 11 | Yes | Yes |
| | Whole | [22] | 48 | 26 | (54%) | 13 | (27%) | 57 | 40 | 97 | — | — |
| | HBV | [23] | 60 | 51 | (85%) | NA | | 156 | NA | 156 | Yes | Yes |
| | Genome | [6] | 426 | 344 | (81%) | 159 | (37%) | 3486 | 739 | 4225 | Yes³ | — |
| | | [24] | 49 | 28 | (57%) | NA | | 121 | 0 | 121 | Yes | Yes |
| | | [25] | 40 | 35 | (90%) | 40 | (100%) | 257 | 1425 | 1682 | Yes | Yes |
| | | [26] | 101 | 94 | (93%) | NA | | 510 | NA | 510 | Yes | Yes |
| | | [27] | 54 | 54 | (100%) | 52 | (96%) | 2870 | 4466 | 7336 | Yes | Yes |
| | DR1-2 | [28] | 40 | 26 | (65%) | 32 | (80%) | 42 | 254 | 296 | Yes | Yes |
| | | this study | 22 | 20 | (91%) | NA | | 27 | NA | 27 | Yes | Yes |
| PCR- | | [29] | 13 | 2 | (15%) | NA | | 2 | NA | 2 | Yes | — |
| based | | [30] | 14 | 14 | (100%) | NA | | 14 | NA | 14 | Yes | Yes |
| | | [31] | 15 | 15 | (100%) | NA | | 15 | NA | 15 | — | Yes |
| | | [32] | 60 | 55 | (92%) | NA | | 60 | NA | 60 | — | Yes |
| | | [33] | 10 | 7 | (70%) | NA | | 8 | NA | 8 | Yes | Yes |
| | | [34] | 60 | 41 | (68%) | 43 | (72%) | 101 | 186 | 287 | Yes | Yes |
| | | [35] | 59⁴ | 45 | (76%) | 6⁵ | (30%) | 45 | 6 | 51 | — | — |
| | | [36] | 15 | 9 | (60%) | NA | | 9 | NA | 9 | — | — |
| | | Total | 1,276 | 1,023 | (81%) | 379 | (50%) | 8,491 | 7,258 | 15,749 | | |

¹HBV (+) HCC cohorts-only;
²three patients overlapping with Jiang 2012 [13] were removed, while the cumulative number of integration sites were compiled and considered unique integration sites due to different reported assay parameters;
³only human chromosome sequence position provided;
⁴cohorts of HBsAg (−)/occult (+) and HBsAg (+) HCC patients;
⁵out of 20 paired non-tumor tissue analyzed;
*denote HBV DNA integration sites;
WGS, whole genome next generation sequencing;
Whole HBV genome, whole HBV genome enrichment was performed prior to NGS;
DR1-2, HBV DR1-2 integration hotspot region was enriched prior to NGS;
Adj. denotes adjacent HCC tumor DNA;
NA denotes not available.

2.3 Clinical Characteristics of HBV-HCC Patients with Integrated HBV DNA

The major clinical factors associated with HCC, such as age, gender, HBV genotype, and whether the HCC arose in a cirrhotic liver, designated as “cirrhotic HCC”, are summarized in Table 12. We categorize HCC patients based on the detectability of integrated HBV DNA in tumor tissue. The general characteristics of the HBV-HCC population [4, 47, 48] are also summarized. Analysis of each parameter was performed as available. The sample sizes that were available for data analysis of each parameter in each cohort are noted in parentheses. Overall, there is no significant difference between the two cohorts as compared to the overall HBV-HCC population for age and gender. The male:female ratio across the cohorts was not significantly different. Of the three reported HBV genotypes, genotype C was the most frequently reported in the integration-detectable tumor cohort (73%), while the tumor cohort with no detectable integration had only 2 patients with genotype reported and both were genotype C. In this cohort, 62% of HCC was derived from the cirrhotic liver in the integration-detectable tumor cohort, which is less than the 70-80% range found in the HBV-HCC population, reported from the literature [4]. 47% of patients with cirrhotic HCCs in the tumor cohort with no detectable integration were reported from 15 patients with available cirrhosis information.

TABLE 12
Overview of the major clinical features of HBV-HCC populations with
and without detectable integrated HBV DNA in tumor tissue.

| | eral | Integrated HBV DNA in study cohort | |
|---|---|---|---|
| | HBV-HCC | Not Detectable | Detectable |
| | populat | (n = 381) | (n = 1,025) |
| Age (years) | | | |
| Range | NA | 33-83 | 11-85 |
| Avg. ± SD | 55-65 ± NA | 59.9 ± 13.3 | 54.9 ± 11.6 |
| | | (n = 37) | (n = 359) |
| Gender (Total) | | (n = 55) | (n = 525) |
| Male | NA | 40 | 395 |
| Female | | 15 | 130 |
| Male/Female | 4:1 | 3.6:1 | 4:1 |
| ratio | | | |
| Genotype (Total) | | (n = 2) | (n = 84) |
| B | NA | 0 | 22 |
| C | | 2 | 61 |
| D | | 0 | 1 |
| Cirrhosis % | 70-90% | 46.7% | 62.3% |
| | (n = NA) | (n = 7/15) | (n = 105/279) |

¹, characteristics of the general HBV-HCC population obtained from the following references [4, 47, 48];
NA denotes not available;
(n) denotes the number of patients available for the analysis;
“Avg. ± SD” denotes average ± standard deviation (SD).

2.4 Recurrent Sites of HBV DNA Integration

Next, we identified RTGs in the compiled HCC cohort and explored their associations with carcinogenesis. Of the 15,749 integration sites examined, 6,249 integration sites were found within 150 kb of gene coding sequences in HCC tumors, and 2,800 genes were identified. Among these 2,800 genes, we considered an integrated gene as a RTG if it was detected from at least two HCC patients and from two independent studies, as described in Materials and Methods. A total of 358 genes were found in 556 HCC patients, constituting 54% of the HBV-HCC patients with detectable HBV integration (n=1,023) and 43% of all HBV-HCC patients (n=1,276) in this cohort. The top 10% of the most frequently recurrent genes (n=36) are listed with summaries of their counts, identified integration sites, and associations with carcinogenesis in Table 13. Interestingly, these 36 genes either have previously suggested associations with carcinogenesis (28/36, 78%) or have no known function (8/36, 22%). As expected, TERT and MLL4 are the two most recurrent genes.

TABLE 13
The top 10% frequently reported recurrent HBV DNA integrated genes
in tumors of HCC patients.

| | Subjects | Junctions | |
|---|---|---|---|
| RTGs | (n) | (n) | Cancer associated [ref] |
| TERT | 257 | 415 | Multiple cancers [49] |
| MLL4 (KMT2B) | 102 | 178 | HCC [50, 51], Spindle cell |
| | | | sarcoma [52], Gastric cancer [53] |
| PLEKHG4B | 38 | 115 | Neuroblastoma [54] |
| LOC100288778 | 34 | 79 | SCLC [55] |
| DDX11L1 | 32 | 56 | Function unknown |
| SNTG1 | 25 | 27 | Lung adenocarcinoma [56] |
| CCNE1 | 23 | 41 | Multiple cancers [57] |
| PGBD2 | 21 | 50 | Function unknown |
| DUX4L4 | 20 | 35 | DUX4 Ewing's sarcoma [58], |
| | | | ALL [59] |
| ROCK1P1 | 19 | 34 | Prostate cancer [60] |
| ANKRD26P1 | 19 | 72 | Breast cancer [61] |
| PARD6G | 18 | 41 | Breast, kidney, liver, lung, ovary, |
| | | | and pancreatic cancers [62] |
| CCNA2 | 18 | 31 | Multiple cancers [63] |
| FAM157A | 14 | 22 | Function unknown |
| CWH43 | 14 | 73 | CRC and TSHomas [64] |
| LOC728323 | 13 | 22 | Oral cancer [65] |
| TPTE | 13 | 30 | HCC [66], prostate cancer [67] |
| FN1 | 13 | 14 | Multiple cancers [68] |
| OR4C6 | 12 | 22 | Pancreatic cancer [69] |
| PRMT2 | 12 | 15 | Glioblastoma [70] |
| ROCK1 | 12 | 23 | HCC [71-74], CRC [75] |
| EMBP1 | 12 | 27 | Oropharyngeal carcinoma [76], |
| | | | multiple primary cancers [77] |
| ANHX | 11 | 16 | Function unknown |
| DDX11L9 | 11 | 16 | Function unknown |
| SENP5 | 11 | 11 | HCC[78], breast cancer [79] |
| ZNF595 | 11 | 14 | Lung cancer [80], Gastric cancer |
| | | | [81] |
| CDRT7 | 10 | 10 | Glioma[82] |
| CTNND2 | 9 | 12 | HCC [83, 84], prostate cancer [85], |
| | | | lung cancer [86] |
| DDX11L5 | 9 | 16 | Function unknown |
| DUX2 | 9 | 9 | Function unknown |
| IL9R | 9 | 45 | HCC[87], lymphoma[88, 89] |
| LINGO2 | 9 | 15 | Gastric cancer[90] |
| PARK2 | 9 | 14 | Colorectal cancer [91] |
| IPCEF1 | 8 | 9 | CLL [92], thyroid cancers [93, 94] |
| LLPH | 8 | 10 | Modulates neuronal growth [95] |
| LOC100505817 | 8 | 11 | Function unknown |

RTGs, integration recurrently targeted genes;
HCC, Hepatocellular carcinoma;
NSCLC, Non-small cell lung cancer;
SCLC, Small cell lung cancer;
ALL, Acute lymphocytic leukemia;
CRC, Colorectal cancer;
TSHoma, Thyrotropin-secreting Pituitary Adenoma;
CLL, Chronic lymphocytic leukemia;
RCC, Renal cell carcinoma.

Next, the 358 RTGs were queried for significantly enriched Gene Ontology (GO) pathways using Enrichr [96]. The top enriched biological pathway of the RTGs was chromatin-mediated maintenance of transcription with a combined score of 17.27 (p<0.05), suggesting possible links with oncogenesis (FIG. 28A). Heparin sulfate-glucosamine 3-sulfotransferase I (HS3ST1) activity was the top enriched pathway from GO molecular functions (FIG. 28B). Sulfotransferases have reported association with carcinogenic activity and HS3ST1 in particular has been implicated in playing a role in inflammation [97]. Lastly, the Drug Signatures Database (DSigbDB) identified trichostatin, that selectively inhibits class I and II histone deacetylase (HDACs), as the drug/compound related to most RTGs, 103 of 358 RTGs examined (FIG. 28C).

2.5 Integration Breakpoints in the HBV Genome

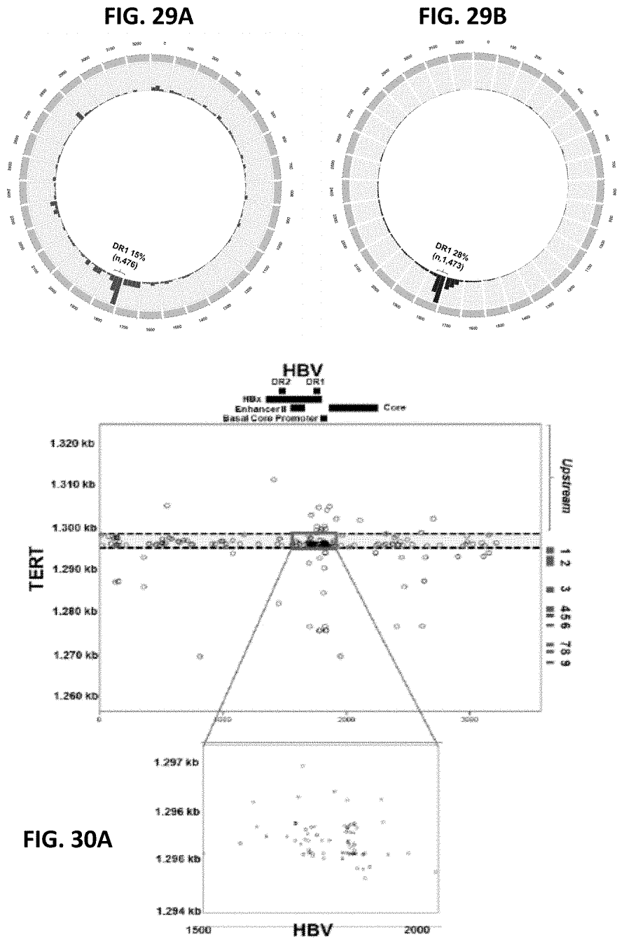

To investigate the distribution patterns of the integration breakpoints in the HBV genomes, we analyzed the HBV breakpoints in tumors (n=3,052) and adj-tumors (n=5,259), where available. We omitted studies that enriched for HBV DR1-2 sequences to assess HBV breakpoints distribution in a non-biased manner. Consistent with previous reports, we observed that 37% of breakpoints were within nt. 1300-1900 region in tumors and 56% in adj-tumors. This region covers the 3′ end of the HBx gene and is where the initiation site of viral replication/transcription are located [6, 23, 27]. Also consistent with previous reporting [16], we observed a breakpoint hotspot in the HBV DR1-2 region, representing 15% for HCC tumors and 28% for adj-tumors of all HBV breakpoints (FIGS. 29A-29B).

2.6 Genomic Breakpoints of TERT, MLL4 and PLEKHG4B RTGs

As HBV integration is believed to be non-sequence-specific, it was of interest to examine all RTG coordinates for similarity to each other. To do so, we plotted the available human and HBV breakpoint coordinates of the three most frequent RTGs identified, TERT, MLL4, and PLEKHG4B (FIGS. 30A-30C).

For TERT, the most frequently recurring RTG, 219 of 415 junctions from 161 HCC patients have both human and HBV breakpoint coordinates available. As expected, most of these breakpoints were centered between DR2 and DR1 of the viral genome and were highly concentrated at the promoter region of the TERT gene (FIG. 30A). Most of the TERT-HBV junctions were unique, supporting the belief that integration occurs mostly in a non-sequence-specific manner. Interestingly, 5 TERT junction sequences of 15 TERT integrations (6.8% of 219 TERT junctions) recurred identically in two or more HCC patients. It should be noted that one of these breakpoints (HBV nt. 1783; Chr5:1275381) was reported from two different studies [14,25] while the remaining four were from one study [27]. Of the 399 available breakpoint coordinates in the TERT gene, 298 (75%) junctions were located upstream of exon 1 and, of these upstream breakpoints, 188 (47%) were located within the TERT promoter region (Chr5:1295162-1296162).

MLL4 is the second most frequently reported RTG with 102 junctions identified from 178 HCC patients studied. Among them, 115 breakpoints from 64 HCC patients have both human and viral coordinates available and are plotted in FIG. 24B. As with TERT, most of the breakpoints were clustered between the DR2 and DR1 of the viral genome and concentrated within exon 3 of the MLL4 gene. There are four identically recurring breakpoints observed in 20 of 115 junctions examined. All four are derived from one study [27], which reported 49 MLL4 junctions.

The third most reported RTG is PLEKHG4B. The reported breakpoints were interestingly all centered within a 3 kb region that is around 131 kb away from the PLEKHG4B coding region. A total of 47 of 116 breakpoints from eight HCC patients have both viral and human coordinates available, as shown in FIG. 24C. All breakpoints were found upstream of the transcription starting site (Chr5:140373). Unlike TERT and MLL4 genes, the viral breakpoints are centered in two HBV regions (nt. 1802-1814 and 2390) at frequencies of 15 and 14, respectively, and at various human coordinates. Further analysis of the human sequences (Chr5:10000-13000) at the integration breakpoint which is upstream of the PLEKHG4B gene, revealed a 1,877 bp simple repeat sequence and a 1,057 bp satellite sequence. Microhomology analysis of this region was searched using 25 nt segments of the HBV genome. No significant homology was identified between the Chr5:10000-13000 region with the two regions, nt. 1802-1818 and 2390 of the HBV genome. Regardless, HBV DNA has been suggested to have a higher propensity to integrate into repeat regions/retrotransposons, as recently shown to occur in vitro upon initial HBV infection by Chauhan et al. [98]. An interesting motif, TAAACCCTAAC, was discovered, appearing four times in the Chr5:10,000-13,000 region and once in the HBV genome, each with p<0.0001. A database search for this motif produced no matches, suggesting further inquiry may be valuable. Motif enrichment analysis of the region for known motifs produced no results. No recurrent breakpoints were identified. Note, 7 of the 8 HCC patients with this unique junction coordinates pattern were reported from one study by Yang et al. [27].

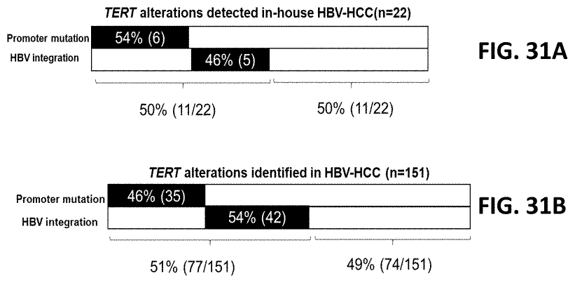

TERT hotspot promoter mutations (−124, −146) are the most frequently reported mutations in HCC, found in about 50% of cases [99-104]. In HBV-HCC, up-regulation of TERT expression could also be caused by HBV integration at or near the TERT promoter region [14, 16, 22, 28, 29, 105]. Next, we compared the incidence of TERT promoter mutation and HBV integration. For our in-house cohort (n=22), shown in FIG. 31A, promoter mutations were found in 6 of 22 samples and integrations in the TERT gene were found in 5 of 22 samples in a mutually exclusive fashion. Together, TERT alterations were detected in 50% (11/22) of this cohort. To expand this mutual exclusive study to a larger sample size, we examined TERT alterations identified by us and others [24,26] together as summarized in FIG. 31B. Of the 151 HBV-HCC patients, 77 (51%) were found to have detectable TERT alterations. 35 of 77 (46%) were by promoter mutations and 42 of 77 (54%) were by integration, in a mutually exclusive manner.

### 3. Discussion

In this study, we compiled and studied over 15,000 HBV DNA integration sites from 1,276 HCC patients reported from 26 previous studies and our in-house study, to test our hypothesis that frequent recurrently targeted genes (RTGs) by HBV integration are HCC driver gene candidates. By using three criteria for RTG identification, we identified 358 RTGs. Encouragingly, the top 10% of the most frequent RTGs (n=36) either have known involvement in carcinogenesis (28/36, 78%) or have unknown function (8/36, 22%). By gene ontology analysis, RTGs were mapped to functions related to carcinogenesis. Together, we demonstrate the potential of HCC driver identification by characterization of frequent RTGs. More studies are needed to define the association of carcinogenesis with the frequency of RTGs.

Three criteria were applied to identify 358 RTGs from HBV integration sites in this study: (1) gene annotation within 150 kb of the breakpoint, the distance previously reported where host genes can be impacted by integration [105,106], (2) reports from ≥2 HCC patients to define “recurrent”, and (3) by ≥2 independent laboratories to avoid the possibility of contamination within a laboratory. We are aware that identification of RTGs across multiple studies is complex in nature, with multi-faceted underlying variables such as integration detection methodologies and patient populations. For instance, some studies do not contain any of 358 RTGs that we identified [35,36], while others have a high detection rate of a particular RTG, such as MLL4 [50], and cMYC [23]. We are also aware that different methodologies for identifying integrations may have different sensitivities that can result in detection of different integration site profiles. Despite these limitations, that may result in missing some RTGs, detection of RTGs constitute a potential HCC driver gene identification that maybe clinically useful for HCC patients.

Encouragingly, the most frequent 10% of RTGs (n=36) identified using the three criteria defined in this study either have known involvement in carcinogenesis (28/36, 78%) or have no known function (8/36, 22%). Although more studies are needed to explore the association of the genes that have unknown functions in hepatocarcinogenesis, of the genes that have known functions, all have been associated with either liver cancer or other cancers. Together with RTG ontology analysis where a significant mapping of genes to functions related to carcinogenesis was observed, our data suggests the potential to not only identify known HCC drivers, but to discover new HCC driver genes by characterization of frequent RTGs for precision disease management. More studies are needed to define the degree of association of carcinogenesis with the frequency of RTGs.

By detailing the three most frequent RTG junction coordinates (TERT, MLL4, and PLEKHG4B), we reveal three important features. First, as expected, the majority of junction coordinates are different, confirming the non-sequence-specific integration in the host genome. The overlapping identical junctions identified in the TERT promoter region highlight the potential importance of the site on impairing the expression of the TERT gene. Second, an interesting pattern was observed in PLEKH4G4B junctions. Although a microhomology search did not suggest the homologous recombination was the cause of this interesting pattern, a highly repetitive sequence, satellite sequences, and a motif of TAAACCCTAAC were identified in these regions. Together suggest possible repeated breakpoints in the region. This supports a possibility of occasional homologous recombination in addition to the non-homologous end-joining mechanism of HBV integration. Since these unique integration pattern sequences was reported from one study and was not reported to be validated in the original tissue DNA, an artifact has not been excluded. Lastly, the mutually exclusive detection of TERT promoter mutations and TERT integration is shown by our small cohort of 22 HCC patients and confirmed by a larger compiled cohort of 151 HCC patients [24,26]. When describing the TERT genetic alterations as an HCC driver, TERT promoter mutations only account for 50% of alterations, indicating the importance of identifying TERT integration. This further emphasizes the need for analysis of frequent RTGs to better characterize HCC.

Most HCC cases develop in a cirrhotic background, though up to 30% of HBV-HCC cases were reported in the absence of cirrhosis (non-cirrhotic HCC) [4,48]. In our study cohort, we identified slightly (but not significant) lower rates (62%) of cirrhotic HCC when integration was detected. In the case of TERT-integrated HCC (n=257) in this study cohort, 51 had information to assess whether the HCC was rising in a cirrhotic background. We identified a significant association (p=0.01) of TERT integrations with cirrhotic HCCs compared to non-cirrhotic HCCs (data not shown). While this cannot be applied to the remaining 206 TERT-integrated HCC patients, in which there was no available information to assess the existence of cirrhosis, it is in line with the association of TERT hotspot promoter mutations with cirrhosis [107].

### 4. Materials and Methods

4.1. Data Mining/Search Strategy

We searched PubMed (2000-Dec. 1, 2018) databases using Medical Subject Heading (MeSH) terms “hepatitis B virus”, “HBV integration”, “hepatitis B integration sites” to identify the literature that have reported HBV integration sites by either NGS- or PCR-based approaches. Additional studies were obtained by cross-referencing from the literature. We included only studies in English and studies that included HCC subjects. We included all studies that identified HBV integration sites using NGS-based approaches. For the studies using PCR-based methods, we only included the studies that analyzed a study sample size of 10 or more HCC patients. HBV integration sites identified by RNA-seq or transcriptome NGS [7, 8, 109] were not included as expression of integrated sequences can be due to many host cellular factors that enable expression of integrated sequences and thus are not within the scope of this study. We filtered out repeated integration sites to ensure each integration site was included only once in our study, with the exception of two studies that utilized different methods on overlapping samples [13,19]. A total of 26 reported studies in addition to our study are included as summarized in Table 2.

4.2. In-House HCC Specimens and HBV Integration Analysis

Archived FFPE tumor tissue DNA (Table 14), as described previously [110,111], from stage I-IIIB patients (n=32) was obtained from the National Cheng-Kung University Medical Center, Taiwan, collected in accordance with the guidelines of the Institutional Review Board. An HBV enrichment NGS assay (JBS Science, Inc) was used. Briefly, NGS libraries were generated, enriched for HBV DR1-2 sequences through two rounds of a multiplex biotinylated HBV primer extension capture (PEC). Libraries were sequenced on the Illumina MiSeq platform (Penn State Hershey Genomics Sciences Facility at Penn State College of Medicine, Hershey, Pa.) and analyzed using ChimericSeq [45] to identify HBV-host junction sequences. Tailored junction-specific PCR-Sanger sequencing was designed and used to validate each HBV integration site of interest, identified by HBV-enriched NGS assay.

TABLE 14
Clinical characteristics of in-house HBV-HCC patient cohort (n = 22).

| | Age | Gender | Cirrhosis | Tumor | Tumor size |
|---|---|---|---|---|---|
| Patient ID | (years) | (M/F) | (−/+) | stage* | (cm) |
| 1 | 71 | M | + | 1 | 3.5 |
| 2 | 68 | M | − | NA | 9.0 |
| 3 | 63 | F | + | NA | 3.7 |
| 4 | 44 | F | − | 1 | 3.5 |
| 5 | 43 | M | − | 2 | 3.0 |
| 6 | 68 | M | − | 1 | 6.5 |
| 7 | 58 | M | − | 2 | 15.0 |
| 8 | 57 | M | − | 1 | 4 |
| 9 | 29 | M | + | 2 | 7 |
| 10 | 41 | M | + | 1 | 2 |
| 11 | 33 | F | + | 1 | 2.5 |
| 12 | 57 | M | + | 1 | 3 |
| 13 | 73 | M | + | 4 | 11.0 |
| 14 | 49 | M | + | 2 | 3.4 |
| 15 | 61 | M | − | 2 | 2.3 |
| 16 | 75 | F | − | 1 | 3.0 |
| 17 | 47 | M | − | 2 | 4.5 |
| 18 | 74 | F | + | 3A | 5.5 |
| 19 | 75 | M | − | 1 | 1.9 |
| 20 | 55 | F | + | 1 | 4.0 |
| 21 | 46 | F | + | 4 | 1.5 |
| 22 | 39 | F | − | 2 | 10 |

*denotes HCC tumors were staged using the tumor-node-metastasis (TNM) staging system.

4.3 TERT Promoter Mutation Analysis by PCR-Sanger Sequencing

HCC tissue DNA was used to amplify a 163-bp region (Chr5:1295151-1295313) of the TERT promoter by using HotStart Plus Taq Polymerase (Qiagen, Valencia, Calif.) with forward primer 5′-CAGCGCTGCCTGAAACTC-3′ (SEQ ID NO: 212) and reverse primer 5′-GTCCTGCCCCTTCACCTT-3′ (SEQ ID NO: 213). The PCR products were sequenced at the NAPCore Facility at the Children's Hospital of Philadelphia (Philadelphia, Pa.) and analyzed using ClustalW software [112].

4.4 Identification of Integration Recurrently Targeted Host Genes (RTGs)

To identify host genes that maybe affected by HBV DNA integration in a universal manner across all studies, we identify the closest gene within 150 kb of the integration event, the distance previously reported where host genes can be impacted by integration [105,106]. To define the status of a RTG, we assessed whether the reported gene was identified in tumors from (A) two or more HCC patients and (B) two or more independent studies to avoid potential cross contamination within a study. The full list of identified RTGs can be provided upon request.

4.5 Gene Functional Enrichment Pathway Analysis

358 RTGs were subjected to enrichment pathway analysis using Enrichr (http://amp.pharm.mssm.edu/Enrichr), to identify significantly (p<0.05) enriched pathways as determined by gene ontology.

### 5. Conclusions

This HBV integration study using an in-house HBV-HCC cohort, in conjunction with previously reported HBV integration sites, allows us to test the hypothesis that HCC drivers can be identified by characterizing frequent recurrent targeted genes (RTGs) by HBV integration. By analyzing over 15,000 HBV integration sites, we bring forth a RTG consensus and demonstrate that characterization of frequent RTGs can be a novel approach to discover or identify HCC drivers for HBV-HCC precision medicine and drug development/discovery.

### REFERENCES

1. Howlader N, N. A., Krapcho M, Miller D, Bishop K, Altekruse S F, Kosary C L, Yu M, Ruhl J, Tatalovich Z, Mariotto A, Lewis D R, Chen H S, Feuer E J, Cronin K A (eds). SEER Cancer Statistics Review, 1975-2013, National Cancer Institute. Bethesda, Md., http://seer.cancer.gov/csr/1975_2013/, based on November 2015 SEER data submission, posted to the SEER web site, April 2016. 2016.
2. American Cancer Society. Cancer Facts & FIGS. 2016. Atlanta: American Cancer Society; 2016.
3. Ferlay J, S. I., Ervik M, Dikshit R, Eser S, Mathers C, Rebelo M, Parkin D M, Forman D, Bray, F. GLOBOCAN 2012 v1.0, Cancer Incidence and Mortality Worldwide: IARC CancerBase No. 11 [Internet]. Lyon, France: International Agency for Research on Cancer; 2013. Available from: http://globocan.iarc.fr, accessed on 14/03/2017. 2013.
4. El-Serag, H. B. Epidemiology of Viral Hepatitis and Hepatocellular Carcinoma. Gastroenterology 2012, 142, 1264-1273.e1261, doi:10.1053/j.gastro.2011.12.061.
5. Tu, T.; Budzinska, M. A.; Vondran, F. W.; Shackel, N. A.; Urban, S. Hepatitis B virus DNA integration occurs early in the viral life cycle in an in vitro infection model via NTCP-dependent uptake of enveloped virus particles. Journal of virology 2018, JVI. 02007-02017.
6. Zhao, L.-H.; Liu, X.; Yan, H.-X.; Li, W.-Y.; Zeng, X.; Yang, Y.; Zhao, J.; Liu, S.-P.; Zhuang, X.-H.; Lin, C. Genomic and oncogenic preference of HBV integration in hepatocellular carcinoma. Nature communications 2016, 7, 12992.
7. Lau, C.-C.; Sun, T.; Ching, A. K.; He, M.; Li, J.-W.; Wong, A. M.; Co, N. N.; Chan, A. W.; Li, P.-S.; Lung, R. W. Viral-human chimeric transcript predisposes risk to liver cancer development and progression. Cancer cell 2014, 25, 335-349.
8. Yoo, S.; Wang, W.; Wang, Q.; Fiel, M. I.; Lee, E.; Hiotis, S. P.; Zhu, J. A pilot systematic genomic comparison of recurrence risks of hepatitis B virus-associated hepatocellular carcinoma with low-and high-degree liver fibrosis. BMC medicine 2017, 15, 214.
9. Chauhan, R.; Churchill, N. D.; Mulrooney-Cousins, P. M.; Michalak, T. I. Initial sites of hepadnavirus integration into host genome in human hepatocytes and in the woodchuck model of hepatitis B-associated hepatocellular carcinoma. Oncogenesis 2017, 6, e317.
10. Bill, C. A.; Summers, J. Genomic DNA double-strand breaks are targets for hepadnaviral DNA integration. Proceedings of the National Academy of Sciences 2004, 101, 11135-11140.
11. Budzinska, M. A.; Shackel, N. A.; Urban, S.; Tu, T. Sequence analysis of integrated hepatitis B virus DNA during HBeAg-seroconversion. Emerging microbes & infections 2018, 7, 142.
12. Lindh, M.; Rydell, G. E.; Larsson, S. B. Impact of integrated viral DNA on the goal to clear hepatitis B surface antigen with different therapeutic strategies. Current opinion in virology 2018, 30, 24-31.
13. Jiang, Z.; Jhunjhunwala, S.; Liu, J.; Haverty, P. M.; Kennemer, M. I.; Guan, Y.; Lee, W.; Carnevali, P.; Stinson, J.; Johnson, S. The effects of hepatitis B virus integration into the genomes of hepatocellular carcinoma patients. Genome Research 2012.
14. Fujimoto, A.; Totoki, Y.; Abe, T.; Boroevich, K. A.; Hosoda, F.; Nguyen, H. H.; Aoki, M.; Hosono, N.; Kubo, M.; Miya, F. Whole-genome sequencing of liver cancers identifies etiological influences on mutation patterns and recurrent mutations in chromatin regulators. Nature genetics 2012, 44, 760-764.
15. Fujimoto, A.; Furuta, M.; Totoki, Y.; Tsunoda, T.; Kato, M.; Shiraishi, Y.; Tanaka, H.; Taniguchi, H.; Kawakami, Y.; Ueno, M. Whole-genome mutational landscape and characterization of noncoding and structural mutations in liver cancer. Nature genetics 2016, 48, 500.
16. Sung, W. K.; Zheng, H.; Li, S.; Chen, R.; Liu, X.; Li, Y.; Lee, N. P.; Lee, W. H.; Ariyaratne, P. N.; Tennakoon, C. Genome-wide survey of recurrent HBV integration in hepatocellular carcinoma. Nature genetics 2012, 44, 765-769.
17. Li, W.; Zeng, X.; Lee, N. P.; Liu, X.; Chen, S.; Guo, B.; Yi, S.; Zhuang, X.; Chen, F.; Wang, G. HMD: an efficient method to detect HBV integration using low coverage sequencing. Genomics 2013, 102, 338-344.
18. Miao, R.; Luo, H.; Zhou, H.; Li, G.; Bu, D.; Yang, X.; Zhao, X.; Zhang, H.; Liu, S.; Zhong, Y. Identification of prognostic biomarkers in hepatitis B virus-related hepatocellular carcinoma and stratification by integrative multi-omics analysis. Journal of hepatology 2014, 61, 840-849.
19. Jhunjhunwala, S.; Jiang, Z.; Stawiski, E. W.; Gnad, F.; Liu, J.; Mayba, O.; Du, P.; Diao, J.; Johnson, S.; Wong, K.-F. Diverse modes of genomic alterations in hepatocellular carcinoma. Genome biology 2014, 15, 436.
20. Hama, N.; Totoki, Y.; Miura, F.; Tatsuno, K.; Saito-Adachi, M.; Nakamura, H.; Arai, Y.; Hosoda, F.; Urushidate, T.; Ohashi, S. Epigenetic landscape influences the liver cancer genome architecture. Nature communications 2018, 9, 1643.
21. Duan, M.; Hao, J.; Cui, S.; Worthley, D. L.; Zhang, S.; Wang, Z.; Shi, J.; Liu, L.; Wang, X.; Ke, A. Diverse modes of clonal evolution in HBV-related hepatocellular carcinoma revealed by single-cell genome sequencing. Cell research 2018, 28, 359.
22. Toh, S. T.; Jin, Y.; Liu, L.; Wang, J.; Babrzadeh, F.; Gharizadeh, B.; Ronaghi, M.; Toh, H. C.; Chow, P. K.-H.; Chung, A. Y. Deep sequencing of the hepatitis B virus in hepatocellular carcinoma patients reveals enriched integration events, structural alterations and sequence variations. Carcinogenesis 2013, 34, 787-798.
23. Yan, H.; Yang, Y.; Zhang, L.; Tang, G.; Wang, Y.; Xue, G.; Zhou, W.; Sun, S. Characterization of the genotype and integration patterns of hepatitis B virus in early-and late-onset hepatocellular carcinoma. Hepatology 2015, 61, 1821-1831.
24. Kawai-Kitahata, F.; Asahina, Y.; Tanaka, S.; Kakinuma, S.; Murakawa, M.; Nitta, S.; Watanabe, T.; Otani, S.; Taniguchi, M.; Goto, F. Comprehensive analyses of mutations and hepatitis B virus integration in hepatocellular carcinoma with clinicopathological features. Journal of gastroenterology 2016, 51, 473-486.
25. Furuta, M.; Tanaka, H.; Shiraishi, Y.; Unida, T.; Imamura, M.; Fujimoto, A.; Fujita, M.; Sasaki-Oku, A.; Maejima, K.; Nakano, K. Characterization of HBV integration patterns and timing in liver cancer and HBV-infected livers. Oncotarget 2018, 9, 25075.
26. Li, C. L.; Li, C. Y.; Lin, Y. Y.; Ho, M. C.; Chen, D. S.; Yeh, S. H.; Chen, P. J. Androgen Receptor Enhances Hepatic TERT Transcription after Hepatitis B Virus Integration or Point Mutation in Promoter Region. Hepatology 2018.
27. Yang, L.; Ye, S.; Zhao, X.; Ji, L.; Zhang, Y.; Zhou, P.; Sun, J.; Guan, Y.; Han, Y.; Ni, C. Molecular Characterization of HBV DNA Integration in Patients with Hepatitis and Hepatocellular Carcinoma. Journal of Cancer 2018, 9, 3225.
28. Ding, D.; Lou, X.; Hua, D.; Yu, W.; Li, L.; Wang, J.; Gao, F.; Zhao, N.; Ren, G.; Li, L. Recurrent Targeted Genes of Hepatitis B Virus in the Liver Cancer Genomes Identified by a Next-Generation Sequencing-Based Approach. PLoS Genetics 2012, 8, e1003065.
29. Ferber, M.; Montoya, D.; Yu, C.; Aderca, I.; McGee, A.; Thorland, E.; Nagorney, D.; Gostout, B.; Burgart, L.; Boix, L. Integrations of the hepatitis B virus (HBV) and human papillomavirus (HPV) into the human telomerase reverse transcriptase (hTERT) gene in liver and cervical cancers. Oncogene 2003, 22, 3813-3820.
30. Wang, Y.; Lau, S. H.; Sham, J. S.-T.; Wu, M.-C.; Wang, T.; Guan, X.-Y. Characterization of HBV integrants in 14 hepatocellular carcinomas: association of truncated X gene and hepatocellular carcinogenesis. Oncogene 2004, 23, 142-148.
31. Tamori, A.; Yamanishi, Y.; Kawashima, S.; Kanehisa, M.; Enomoto, M.; Tanaka, H.; Kubo, S.; Shiomi, S.; Nishiguchi, S. Alteration of gene expression in human hepatocellular carcinoma with integrated hepatitis B virus DNA. Clinical Cancer Research 2005, 11, 5821-5826.
32. Murakami, Y.; Saigo, K.; Takashima, H.; Minami, M.; Okanoue, T.; Brechot, C.; Paterlini-Brechot, P. Large scaled analysis of hepatitis B virus (HBV) DNA integration in HBV related hepatocellular carcinomas. Gut 2005, 54, 1162-1168.
33. Saigo, K.; Yoshida, K.; Ikeda, R.; Sakamoto, Y.; Murakami, Y.; Urashima, T.; Asano, T.; Kenmochi, T.; Inoue, I. Integration of hepatitis B virus DNA into the myeloid/lymphoid or mixed-lineage leukemia (MLL4) gene and rearrangements of MLL4 in human hepatocellular carcinoma. Human mutation 2008, 29, 703-708.
34. Jiang, S.; Yang, Z.; Li, W.; Li, X.; Wang, Y.; Zhang, J.; Xu, C.; Chen, P. J.; Hou, J.; McCrae, M. A. Re-evaluation of the Carcinogenic Significance of Hepatitis B Virus Integration in Hepatocarcinogenesis. PloS one 2012, 7, e40363.
35. Saitta, C.; Tripodi, G.; Barbera, A.; Bertuccio, A.; Smedile, A.; Ciancio, A.; Raffa, G.; Sangiovanni, A.; Navarra, G.; Raimondo, G. Hepatitis B virus (HBV) DNA integration in patients with occult HBV infection and hepatocellular carcinoma. Liver International 2015, 35, 2311-2317.
36. Fang, X.; Wu, H.-H.; Ren, J.-J.; Liu, H.-Z.; Li, K.-Z.; Li, J.-L.; Tang, Y.-P.; Xiao, C.-C.; Huang, T.-R.; Deng, W. Associations between serum HBX quasispecies and their integration in hepatocellular carcinoma. Int J Clin Exp Pathol 2017, 10, 11857-11866.
37. Scotto, J.; Hadchouel, M.; Hery, C.; Alvarez, F.; Yvart, J.; Tiollais, P.; Bernard, O.; Brechot, C. Hepatitis B virus DNA in children's liver diseases: detection by blot hybridisation in liver and serum. Gut 1983, 24, 618-624.
38. Huang, H. P. O.; Tsuei, D. A. W. J. E. N.; Wang, K. J. A. N.; Chen, Y. L.; Ni, Y. E. N. H.; Jeng, Y. M.; Chen, H. L.; Hsu, H. Y.; Chang, M. E. I. H. Differential integration rates of hepatitis B virus DNA in the liver of children with chronic hepatitis B virus infection and hepatocellular carcinoma. Journal of gastroenterology and hepatology 2005, 20, 1206-1214.
39. Shafritz, D. A.; Shouval, D.; Sherman, H. I.; Hadziyannis, S. J.; Kew, M. C. Integration of hepatitis B virus DNA into the genome of liver cells in chronic liver disease and hepatocellular carcinoma. New England Journal of Medicine 1981, 305, 1067-1073.
40. Koshy, R.; Maupas, P.; Müller, R.; Hofschneider, P. Detection of hepatitis B virus-specific DNA in the genomes of human hepatocellular carcinoma and liver cirrhosis tissues. The Journal of general virology 1981, 57, 95.
41. Takada, S.; Gotoh, Y.; Hayashi, S.; Yoshida, M.; Koike, K. Structural rearrangement of integrated hepatitis B virus DNA as well as cellular flanking DNA is present in chronically infected hepatic tissues. Journal of virology 1990, 64, 822-828.
42. Hai, H.; Tamori, A.; Kawada, N. Role of hepatitis B virus DNA integration in human hepatocarcinogenesis. World journal of gastroenterology: WJG 2014, 20, 6236.
43. Hu, B.; Wang, R.; Fu, J.; Su, M.; Du, M.; Liu, Y.; Li, H.; Wang, H.; Lu, F.; Jiang, J. Integration of hepatitis B virus S gene impacts on hepatitis B surface antigen levels in patients with antiviral therapy. Journal of gastroenterology and hepatology 2018, 33, 1389-1396.
44. Larsson, S.; Tripodi, G.; Raimondo, G.; Saitta, C.; Norkrans, G.; Pollicino, T.; Lindh, M. Integration of hepatitis B virus DNA in chronically infected patients assessed by Alu-PCR. Journal of Medical Virology 2018.
45. Shieh, F.-S.; Jongeneel, P.; Steffen, J. D.; Lin, S.; Jain, S.; Song, W.; Su, Y.-H. ChimericSeq: An open-source, user-friendly interface for analyzing NGS data to identify and characterize viral-host chimeric sequences. PLOS ONE 2017, 12, e0182843, doi:10.1371/journal.pone.0182843.
46. Li, X.; Zhang, J.; Yang, Z.; Kang, J.; Jiang, S.; Zhang, T.; Chen, T.; Li, M.; Lv, Q.; Chen, X. The function of targeted host genes determines the oncogenicity of HBV integration in hepatocellular carcinoma. Journal of hepatology 2014, 60, 975-984.
47. Yang, J. D.; Kim, W.; Coelho, R.; Mettler, T. A.; Benson, J. T.; Sanderson, S. O.; Therneau, T. M.; Kim, B.; Roberts, L. R. Cirrhosis is present in most patients with hepatitis B and hepatocellular carcinoma. Clinical Gastroenterology and Hepatology 2011, 9, 64-70.
48. El-Serag, H. B.; Rudolph, K. L. Hepatocellular carcinoma: epidemiology and molecular carcinogenesis. Gastroenterology 2007, 132, 2557-2576.
49. Heidenreich, B.; Rachakonda, P. S.; Hemminki, K.; Kumar, R. TERT promoter mutations in cancer development. Current opinion in genetics & development 2014, 24, 30-37.
50. Saigo, K.; Yoshida, K.; Ikeda, R.; Sakamoto, Y.; Murakami, Y.; Urashima, T.; Asano, T.; Kenmochi, T.; Inoue, I. Integration of hepatitis B virus DNA into the myeloid/lymphoid or mixed-lineage leukemia (MLL4) gene and rearrangements of MLL4 in human hepatocellular carcinoma. Human Mutation 2008, 29, 703-708, doi:10.1002/humu.20701.
51. Tamori, A.; Nishiguchi, S.; Shiomi, S.; Hayashi, T.; Kobayashi, S.; Habu, D.; Takeda, T.; Seki, S.; Hirohashi, K.; Tanaka, H., et al. Hepatitis B Virus DNA Integration in Hepatocellular Carcinoma After Interferon-Induced Disappearance of Hepatitis C Virus. The American Journal Of Gastroenterology 2005, 100, 1748, doi:10.1111/j.1572-0241.2005.41914.x.
52. O'Meara, E.; Stack, D.; Phelan, S.; McDonagh, N.; Kelly, L.; Sciot, R.; Debiec-Rychter, M.; Morris, T.; Cochrane, D.; Sorensen, P., et al. Identification of an MLL4-GPS2 fusion as an oncogenic driver of undifferentiated spindle cell sarcoma in a child. Genes, Chromosomes and Cancer 2014, 53, 991-998, doi:doi:10.1002/gcc.22208.
53. Pan, X.; Ji, X.; Zhang, R.; Zhou, Z.; Zhong, Y.; Peng, W.; Sun, N.; Xu, X.; Xia, L.; Li, P., et al. Landscape of somatic mutations in gastric cancer assessed using next-generation sequencing analysis. Oncology letters 2018, 16, 4863-4870, doi:10.3892/ol.2018.9314.
54. Chicard, M.; Boyault, S.; Daage, L. C.; Richer, W.; Gentien, D.; Pierron, G.; Lapouble, E.; Bellini, A.; Clement, N.; Iacono, I. Genomic copy number profiling using circulating free tumor DNA highlights heterogeneity in neuroblastoma. Clinical Cancer Research 2016, clincanres. 0500.2016.
55. Li, J.; Wang, J.; Chen, Y.; Yang, L.; Chen, S. A prognostic 4-gene expression signature for squamous cell lung carcinoma. Journal of cellular physiology 2017, 232, 3702-3713.
56. Meng, F.; Zhang, L.; Ren, Y.; Ma, Q. The genomic alterations of lung adenocarcinoma and lung squamous cell carcinoma can explain the differences of their overall survival rates. Journal of Cellular Physiology 0, doi:doi:10.1002/jcp.27917.
57. Donnellan, R.; Chetty, R. Cyclin E in human cancers. The FASEB Journal 1999, 13, 773-780.
58. Yoshimoto, T.; Tanaka, M.; Homme, M.; Yamazaki, Y.; Takazawa, Y.; Antonescu, C. R.; Nakamura, T. CIC-DUX4 Induces Small Round Cell Sarcomas Distinct from Ewing Sarcoma. Cancer Res 2017, 77, 2927-2937, doi:10.1158/0008-5472.Can-16-3351.
59. Yasuda, T.; Tsuzuki, S.; Kawazu, M.; Hayakawa, F.; Kojima, S.; Ueno, T.; Imoto, N.; Kohsaka, S.; Kunita, A.; Doi, K., et al. Recurrent DUX4 fusions in B cell acute lymphoblastic leukemia of adolescents and young adults. Nature Genetics 2016, 48, 569, doi:10.1038/ng.3535 https://www.nature.com/articles/ng.3535#supplementary-information.
60. Luo, Y.; Jiang, Q.-W.; Wu, J.-Y.; Qiu, J.-G.; Zhang, W.-J.; Mei, X.-L.; Shi, Z.; Di, J.-M. Regulation of migration and invasion by Toll-like receptor-9 signaling network in prostate cancer. Oncotarget 2015, 6, 22564.
61. Krepischi, A. C.; Achatz, M. I.; Santos, E. M.; Costa, S. S.; Lisboa, B. C.; Brentani, H.; Santos, T. M.; Goncalves, A.; Nobrega, A. F.; Pearson, P. L., et al. Germline DNA copy number variation in familial and early-onset breast cancer. Breast cancer research: BCR 2012, 14, R24, doi:10.1186/bcr3109.
62. Marques, E.; Englund, J. I.; Tervonen, T. A.; Virkunen, E.; Laakso, M.; Myllynen, M.; Mäkelä, A.; Ahvenainen, M.; Lepikhova, T.; Monni, O., et al. Par6G suppresses cell proliferation and is targeted by loss-of-function mutations in multiple cancers. Oncogene 2015, 35, 1386, doi:10.1038/onc.2015.196 https://www.nature.com/articles/onc2015196#supplementary-information.
63. Otto, T.; Sicinski, P. Cell cycle proteins as promising targets in cancer therapy. Nature Reviews Cancer 2017, 17, 93.
64. Chu, C.-M.; Yao, C.-T.; Chang, Y.-T.; Chou, H.-L.; Chou, Y.-C.; Chen, K.-H.; Terng, H.-J.; Huang, C.-S.; Lee, C.-C.; Su, S.-L., et al. Gene expression profiling of colorectal tumors and normal mucosa by microarrays meta-analysis using prediction analysis of microarray, artificial neural network, classification, and regression trees. Disease markers 2014, 2014, 634123-634123, doi:10.1155/2014/634123.
65. Ambatipudi, S.; Gerstung, M.; Gowda, R.; Pai, P.; Borges, A. M.; Schïffer, A. A.; Beerenwinkel, N.; Mahimkar, M. B. Genomic Profiling of Advanced-Stage Oral Cancers Reveals Chromosome 11q Alterations as Markers of Poor Clinical Outcome. PLOS ONE 2011, 6, e17250, doi:10.1371/journal.pone.0017250.
66. Dong, X. Y.; Su, Y. R.; Qian, X. P.; Yang, X. A.; Pang, X. W.; Wu, H. Y.; Chen, W. F. Identification of two novel CT antigens and their capacity to elicit antibody response in hepatocellular carcinoma patients. British Journal Of Cancer 2003, 89, 291, doi:10.1038/sj.bjc.6601062.
67. Singh, A. P.; Bafna, S.; Chaudhary, K.; Venkatraman, G.; Smith, L.; Eudy, J. D.; Johansson, S. L.; Lin, M.-F.; Batra, S. K. Genome-wide expression profiling reveals transcriptomic variation and perturbed gene networks in androgen-dependent and androgen-independent prostate cancer cells. Cancer Letters 2008, 259, 28-38, doi:https://doi.org/10.1016/j.canlet.2007.09.018.
68. Hynes, R. O. Fibronectins; Springer Science & Business Media: 2012.
69. Weber, L.; Massberg, D.; Becker, C.; Altmuller, J.; Ubrig, B.; Bonatz, G.; Wolk, G.; Philippou, S.; Tannapfel, A.; Hatt, H., et al. Olfactory Receptors as Biomarkers in Human Breast Carcinoma Tissues. Frontiers in oncology 2018, 8, 33, doi:10.3389/fonc.2018.00033.
70. Dong, F.; Li, Q.; Yang, C.; Huo, D.; Wang, X.; Ai, C.; Kong, Y.; Sun, X.; Wang, W.; Zhou, Y., et al. PRMT2 links histone H3R8 asymmetric dimethylation to oncogenic activation and tumorigenesis of glioblastoma. Nat Commun 2018, 9, 4552, doi:10.1038/s41467-018-06968-7.
71. Tang, J.; Liu, C.; Xu, B.; Wang, D.; Ma, Z.; Chang, X. ARHGEF10L contributes to liver tumorigenesis through RhoA-ROCK1 signaling and the epithelial-mesenchymal transition. Experimental cell research 2019, 374, 46-68, doi:10.1016/j.yexcr.2018.11.007.
72. Liu, W.; Zhang, Q.; Tang, Q.; Hu, C.; Huang, J.; Liu, Y.; Lu, Y.; Wang, Q.; Li, G.; Zhang, R. Lycorine inhibits cell proliferation and migration by inhibiting ROCK1/cofilininduced actin dynamics in HepG2 hepatoblastoma cells. Oncology reports 2018, 40, 2298-2306, doi:10.3892/or.2018.6609.
73. Ding, W.; Tan, H.; Zhao, C.; Li, X.; Li, Z.; Jiang, C.; Zhang, Y.; Wang, L. MiR-145 suppresses cell proliferation and motility by inhibiting ROCK1 in hepatocellular carcinoma. Tumour biology: the journal of the International Society for Oncodevelopmental Biology and Medicine 2016, 37, 6255-6260, doi:10.1007/s13277-015-4462-3.
74. Deng, Q.; Xie, L.; Li, H. MiR-506 suppresses cell proliferation and tumor growth by targeting Rho-associated protein kinase 1 in hepatocellular carcinoma. Biochem Biophys Res Commun 2015, 467, 921-927, doi:10.1016/j.bbrc.2015.10.043.
75. Song, G. L.; Jin, C. C.; Zhao, W.; Tang, Y.; Wang, Y. L.; Li, M.; Xiao, M.; Li, X.; Li, Q. S.; Lin, X., et al. Regulation of the RhoA/ROCK/AKT/beta-catenin pathway by arginine-specific ADP-ribosytransferases 1 promotes migration and epithelial-mesenchymal transition in colon carcinoma. International journal of oncology 2016, 49, 646-656, doi:10.3892/ijo.2016.3539.
76. Ren, S.; Gaykalova, D.; Wang, J.; Guo, T.; Danilova, L.; Favorov, A.; Fertig, E.; Bishop, J.; Khan, Z.; Flam, E., et al. Discovery and development of differentially methylated regions in human papillomavirus-related oropharyngeal squamous cell carcinoma. Int J Cancer 2018, 143, 2425-2436, doi:10.1002/ijc.31778.
77. Park, S. L.; Caberto, C. P.; Lin, Y.; Goodloe, R. J.; Dumitrescu, L.; Love, S. A.; Matise, T. C.; Hindorff, L. A.; Fowke, J. H.; Schumacher, F. R., et al. Association of cancer susceptibility variants with risk of multiple primary cancers: The population architecture using genomics and epidemiology study. Cancer Epidemiol Biomarkers Prev 2014, 23, 2568-2578, doi:10.1158/1055-9965.Epi-14-0129.
78. Jin, Z. L.; Pei, H.; Xu, Y. H.; Yu, J.; Deng, T. The SUMO-specific protease SENPS controls DNA damage response and promotes tumorigenesis in hepatocellular carcinoma. European review for medical and pharmacological sciences 2016, 20, 3566-3573.
79. Cashman, R.; Cohen, H.; Ben-Hamo, R.; Zilberberg, A.; Efroni, S. SENPS mediates breast cancer invasion via a TGFbetaRI SUMOylation cascade. Oncotarget 2014, 5, 1071-1082, doi:10.18632/oncotarget.1783.
80. Kanwal, M.; Ding, X. J.; Ma, Z. H.; Li, L. W.; Wang, P.; Chen, Y.; Huang, Y. C.; Cao, Y. Characterization of germline mutations in familial lung cancer from the Chinese population. Gene 2018, 641, 94-104, doi:10.1016/j.gene.2017.10.020.
81. Cui, J.; Yin, Y.; Ma, Q.; Wang, G.; Olman, V.; Zhang, Y.; Chou, W. C.; Hong, C. S.; Zhang, C.; Cao, S., et al. Comprehensive characterization of the genomic alterations in human gastric cancer. Int J Cancer 2015, 137, 86-95, doi:10.1002/ijc.29352.
82. Hu, C.; Zhou, Y.; Liu, C.; Kang, Y. Risk assessment model constructed by differentially expressed lncRNAs for the prognosis of glioma. Oncology reports 2018, 40, 2467-2476, doi:10.3892/or.2018.6639.
83. Chen, R.; Dong, Y.; Xie, X.; Chen, J.; Gao, D.; Liu, Y.; Ren, Z.; Cui, J. Screening candidate metastasis-associated genes in three-dimensional HCC spheroids with different metastasis potential. International journal of clinical and experimental pathology 2014, 7, 2527-2535.
84. Huang, F.; Chen, J.; Lan, R.; Wang, Z.; Chen, R.; Lin, J.; Fu, L. Hypoxia induced delta-Catenin to enhance mice hepatocellular carcinoma progression via Wnt signaling. Experimental cell research 2019, 374, 94-103, doi:10.1016/j.yexcr.2018.11.011.
85. Zhang, P.; Schaefer-Klein, J.; Cheville, J. C.; Vasmatzis, G.; Kovtun, I. V. Frequently rearranged and overexpressed delta-catenin is responsible for low sensitivity of prostate cancer cells to androgen receptor and beta-catenin antagonists. Oncotarget 2018, 9, 24428-24442, doi:10.18632/oncotarget.25319.
86. Huang, F.; Chen, J.; Wang, Z.; Lan, R.; Fu, L.; Zhang, L. delta-Catenin promotes tumorigenesis and metastasis of lung adenocarcinoma. Oncology reports 2018, 39, 809-817, doi:10.3892/or.2017.6140.
87. Li, H. J.; Sun, Q. M.; Liu, L. Z.; Zhang, J.; Huang, J.; Wang, C. H.; Ding, R.; Song, K.; Tong, Z. High expression of IL-9R promotes the progression of human hepatocellular carcinoma and indicates a poor clinical outcome. Oncology reports 2015, 34, 795-802, doi:10.3892/or.2015.4060.
88. Renauld, J.-C. IL-9 and its Receptor: From Signal Transduction to Tumorigenesis AU—Knoops, Laurent. Growth Factors 2004, 22, 207-215, doi:10.1080/08977190410001720879.
89. Lv, X.; Feng, L.; Fang, X.; Jiang, Y.; Wang, X. Overexpression of IL-9 receptor in diffuse large B-cell lymphoma. International journal of clinical and experimental pathology 2013, 6, 911-916.
90. Jo, J. H.; Park, S. B.; Park, S.; Lee, H. S.; Kim, C.; Jung, D. E.; Song, S. Y. Novel Gastric Cancer Stem Cell-Related Marker LINGO2 Is Associated with Cancer Cell Phenotype and Patient Outcome. Int J Mol Sci 2019, 20, doi:10.3390/ijms20030555.
91. Bhat, Z. I.; Kumar, B.; Bansal, S.; Naseem, A.; Tiwari, R. R.; Wahabi, K.; Sharma, G. D.; Alam Rizvi, M. M. Association of PARK2 promoter polymorphisms and methylation with colorectal cancer in North Indian population. Gene 2019, 682, 25-32, doi:10.1016/j.gene.2018.10.010.
92. Speedy, H. E.; Di Bernardo, M. C.; Sava, G. P.; Dyer, M. J.; Holroyd, A.; Wang, Y.; Sunter, N.J.; Mansouri, L.; Juliusson, G.; Smedby, K. E., et al. A genome-wide association study identifies multiple susceptibility loci for chronic lymphocytic leukemia. Nat Genet 2014, 46, 56-60, doi:10.1038/ng.2843.
93. Passon, N.; Bregant, E.; Sponziello, M.; Dima, M.; Rosignolo, F.; Durante, C.; Celano, M.; Russo, D.; Filetti, S.; Damante, G. Somatic amplifications and deletions in genome of papillary thyroid carcinomas. Endocrine 2015, 50, 453-464, doi:10.1007/s12020-015-0592-z.
94. Schulten, H. J.; Al-Mansouri, Z.; Baghallab, I.; Bagatian, N.; Subhi, O.; Karim, S.; Al-Aradati, H.; Al-Mutawa, A.; Johary, A.; Meccawy, A. A., et al. Comparison of microarray expression profiles between follicular variant of papillary thyroid carcinomas and follicular adenomas of the thyroid. BMC genomics 2015, 16 Suppl 1, S7, doi:10.1186/1471-2164-16-s1-s7.
95. Yu, N. K.; Kim, H. F.; Shim, J.; Kim, S.; Kim, D. W.; Kwak, C.; Sim, S. E.; Choi, J. H.; Ahn, S.; Yoo, J., et al. A transducible nuclear/nucleolar protein, mLLP, regulates neuronal morphogenesis and synaptic transmission. Sci Rep 2016, 6, 22892, doi:10.1038/srep22892.
96. Kuleshov, M. V.; Jones, M. R.; Rouillard, A. D.; Fernandez, N. F.; Duan, Q.; Wang, Z.; Koplev, S.; Jenkins, S. L.; Jagodnik, K. M.; Lachmann, A. Enrichr: a comprehensive gene set enrichment analysis web server 2016 update. Nucleic acids research 2016, 44, W90-W97.
97. Smits, N.C.; Kobayashi, T.; Srivastava, P. K.; Skopelja, S.; Ivy, J. A.; Elwood, D. J.; Stan, R. V.; Tsongalis, G. J.; Sellke, F. W.; Gross, P. L. HS3ST1 genotype regulates antithrombin's inflammomodulatory tone and associates with atherosclerosis. Matrix Biology 2017, 63, 69-90.
98. Chauhan, R.; Shimizu, Y.; Watashi, K.; Wakita, T.; Fukasawa, M.; Michalak, T. I. Retrotransposon Elements among Initial Sites of Hepatitis B Virus Integration into Human Genome in the HepG2-NTCP Cell Infection Model. Cancer genetics 2019.
99. Nault, J. C.; Calderaro, J.; Di Tommaso, L.; Balabaud, C.; Zafrani, E. S.; Bioulac-Sage, P.; Roncalli, M.; Zucman-Rossi, J. Telomerase reverse transcriptase promoter mutation is an early somatic genetic alteration in the transformation of premalignant nodules in hepatocellular carcinoma on cirrhosis. Hepatology 2014, 60, 1983-1992.
100. Nault, J. C.; Mallet, M.; Pilati, C.; Calderaro, J.; Bioulac-Sage, P.; Laurent, C.; Laurent, A.; Cherqui, D.; Balabaud, C.; Zucman-Rossi, J. High frequency of telomerase reverse-transcriptase promoter somatic mutations in hepatocellular carcinoma and preneoplastic lesions. Nature communications 2013, 4, 2218.
101. Nault, J.-C.; Zucman-Rossi, J. Genetics of hepatocellular carcinoma: the next generation. Journal of hepatology 2014, 60, 224-226.
102. Pinyol, R.; Tovar, V.; Llovet, J. M. TERT promoter mutations: gatekeeper and driver of hepatocellular carcinoma. Journal of hepatology 2014, 61, 685.
103. Quaas, A.; Oldopp, T.; Tharun, L.; Klingenfeld, C.; Krech, T.; Sauter, G.; Grob, T. J. Frequency of TERT promoter mutations in primary tumors of the liver. Virchows Archiv 2014, 465, 673-677.
104. Totoki, Y.; Tatsuno, K.; Covington, K. R.; Ueda, H.; Creighton, C. J.; Kato, M.; Tsuji, S.; Donehower, L. A.; Slagle, B. L.; Nakamura, H. Trans-ancestry mutational landscape of hepatocellular carcinoma genomes. Nature genetics 2014, 46, 1267.
105. Horikawa, I.; Barrett, J. C. cis-Activation of the human telomerase gene (hTERT) by the hepatitis B virus genome. Journal of the National Cancer Institute 2001, 93, 1171-1173.
106. Shamay, M.; Agami, R.; Shaul, Y. HBV integrants of hepatocellular carcinoma cell lines contain an active enhancer. Oncogene 2001, 20, 6811.
107. Chen, Y.-L.; Jeng, Y.-M.; Chang, C.-N.; Lee, H.-J.; Hsu, H.-C.; Lai, P.-L.; Yuan, R.-H. TERT promoter mutation in resectable hepatocellular carcinomas: a strong association with hepatitis C infection and absence of hepatitis B infection. International Journal of Surgery 2014, 12, 659-665.
108. Liu, C.-J.; Kao, J.-H. Global perspective on the natural history of chronic hepatitis B: role of hepatitis B virus genotypes A to J. In Proceedings of Seminars in liver disease; pp. 097-102.
109. Dong, H.; Zhang, L.; Qian, Z.; Zhu, X.; Zhu, G.; Chen, Y.; Xie, X.; Ye, Q.; Zang, J.; Ren, Z. Identification of HBV-MLL4 Integration and Its Molecular Basis in Chinese Hepatocellular Carcinoma. 2015.
110. Lin, S. Y.; Dhillon, V.; Jain, S.; Chang, T.-T.; Hu, C.-T.; Lin, Y.-J.; Chen, S.-H.; Chang, K.-C.; Song, W.; Yu, L. A locked nucleic acid clamp-mediated PCR assay for detection of a p53 codon 249 hotspot mutation in urine. The Journal of Molecular Diagnostics 2011, 13, 474-484.
111. Jain, S.; Xie, L.; Boldbaatar, B.; Lin, S. Y.; Hamilton, J. P.; Meltzer, S. J.; Chen, S.-H.; Hu, C.-T.; Block, T. M.; Song, W., et al. Differential methylation of the promoter and first exon of the RASSF1A gene in hepatocarcinogenesis. Hepatology Research 2015, 10.1111/hepr.12449, doi:10.1111/hepr.12449.
112. Madeira, F.; Lee, J.; Buso, N.; Gur, T.; Madhusoodanan, N.; Basutkar, P.; Tivey, A.; Potter, S. C.; Finn, R. D.; Lopez, R. The EMBL-EBI search and sequence analysis tools APIs in 2019. Nucleic acids research 2019.
113. Su Y H, Wang M, Brenner D E, Ng A, Melkonyan H, Umansky S, et al. Human urine contains small, 150 to 250 nucleotide-sized, soluble DNA derived from the circulation and may be useful in the detection of colorectal cancer. Journal of Molecular Diagnostics 2004; 6:101-107.
114. Su Y H, Song J, Wang Z, Wang X, Wang M, Brenner D E, et al. Removal of high molecular weight DNA by carboxylated magnetic beads enhances the detection of mutated K-ras DNA in urine. Annals of the New York Academy of Sciences 2008; 1137:82-91.
115. Gnirke A, Melnikov A, Maguire J, Rogov P, LeProust E M, Brockman W, et al. Solution hybrid selection with ultra-long oligonucleotides for massively parallel targeted sequencing. Nature biotechnology 2009; 27:182-189.
116. Ozawa T, Itoyama T, Sadamori N, Yamada Y, Hata T, Tomonaga M, et al. Rapid isolation of viral integration site reveals frequent integration of HTLV-1 into expressed loci. Journal of human genetics 2004; 49:154-165.
117. Yamamoto M, Cid E, Bru S, Yamamoto F. Rare and frequent promoter methylation, respectively, of TSHZ2 and 3 genes that are both downregulated in expression in breast and prostate cancers. 2011.
118. Wang W, Zhao L J, Tan Y-X, Ren H, Qi Z-T. Identification of deregulated miRNAs and their targets in hepatitis B virus-associated hepatocellular carcinoma. World journal of gastroenterology: WJG 2012; 18:5442.
119. Harel S A, Ben-Moshe N B, Aylon Y, Bublik D, Moskovits N, Toperoff G, et al. Reactivation of epigenetically silenced miR-512 and miR-373 sensitizes lung cancer cells to cisplatin and restricts tumor growth. Cell Death & Differentiation. 2015.

1. A method for identifying at least one HBV-host junction sequence (HBV-JS) from a biological sample of a subject, comprising:
preparing a DNA sample from the biological sample;
performing at least one round of enrichment over the DNA sample, each round comprising:
capturing, by means of an HBV probe set, HBV DNA sequence-containing DNA molecules from the DNA sample, wherein the HBV probe set comprises a plurality of HBV primers having sequences thereof selectively and respectively corresponding to different regions of an HBV genome, and each labelled with an immobilization portion configured to allow immobilization onto a solid support.

2. The method of claim 1, wherein the capturing, by means of an HBV probe set, HBV DNA sequence-containing DNA molecules from the DNA sample is through a primer extension capture assay, comprising:
denaturing the DNA sample to thereby obtain a denatured DNA sample;
contacting the plurality of HBV primers with the denatured DNA sample for annealing;
performing a primer extension reaction;
immobilizing the DNA molecules captured by the plurality of HBV primers; and
eluting the DNA molecules.

3. The method of claim 1, wherein each of the at least one round of enrichment further comprises:
amplifying the DNA molecules.

4. The method of claim 1, wherein each of the plurality of HBV primers comprises a sequence selected from a group consisting of SEQ ID NOS: 49-175.

5. The method of claim 1, wherein the preparing a DNA sample from the biological sample comprises:
constructing a DNA library from the biological sample.

6. The method of claim 5, wherein the DNA library is an ssDNA library.

7. The method of claim 1, wherein a number of the at least one round of enrichment is more than one.

8. The method of claim 1, wherein the biological sample is a body fluid sample.

9. The method of claim 8, wherein the biological sample is a urine sample.

10. The method of claim 1, wherein in the preparing a DNA sample from the biological sample, each DNA molecule obtained thereby comprises a pair of adaptors flanking a DNA fragment from the subject, wherein in the capturing, by means of an HBV probe set, HBV DNA sequence-containing DNA molecules from the DNA sample, the DNA molecules are captured in presence of at least one adaptor blocker configured to hybridize with sequences corresponding to the pair of adaptors in the each DNA molecule so as to minimize off-target capture.

11. A kit for identifying at least one HBV-host junction sequence (HBV-JS) from a biological sample of a subject, comprising:
an HBV probe set, comprising a plurality of HBV primers having sequences thereof selectively and respectively corresponding to different regions of an HBV genome, each labelled with an immobilization portion; and
a solid support, conjugated with a coupling partner on a surface thereof, wherein the coupling partner is configured to form a secure coupling to the immobilization portion of each HBV primer to thereby allow immobilization of HBV DNA sequence-containing DNA molecules to the solid support.

12. The kit according to claim 11, wherein each of the plurality of HBV primers comprises a sequence selected from a group consisting of SEQ ID NOS: 49-175.

13. The kit according to claim 11, further comprising a pair of adaptors, configured to be ligated to two ends of each DNA molecule in the biological sample to thereby obtain a DNA library from the biological sample.

14. The kit according to claim 13, further comprising at least one adaptor blocker configured to hybridize with sequences corresponding to the pair of adaptors in the each DNA molecule in the DNA library so as to minimize off-target capture.

15. The kit according to claim 13, wherein the DNA library is a single-stranded DNA library.

16. The kit according to claim 11, further comprising at least one pair of amplifying primers, configured to amplify the HBV DNA sequence-containing DNA molecules.

17. The kit according to claim 11, wherein:
the immobilization portion comprises a biotin moiety; and
the coupling partner comprises at least one of streptavidin, avidin, or an anti-biotin antibody.

18. The kit according to claim 17, wherein the solid support comprises streptavidin magnetic beads.

19. The kit according to claim 11, further comprising a software for identifying the at least one HBV-JS from data obtained from a sequencing assay over the HBV DNA sequence-containing DNA molecules.

20. The method of claim 19, wherein the software is ChimericSeq.


SEQUENCE LISTING (213 sequences)

SEQ ID NO: 1 - DNA, 21 bp, Artificial Sequence
  synthesized
  phosphorothioate bond (19)..(20)
  phosphorothioate bond (20)..(21)
caagcagaagacggcatacga

SEQ ID NO: 2 - DNA, 20 bp, Artificial Sequence
  synthesized
  phosphorothioate bond (18)..(19)
  phosphorothioate bond (19)..(20)
aatgatacggcgaccaccga

SEQ ID NO: 3 - DNA, 11 bp, Artificial Sequence
  synthesized
  locked nucleic acid nucleotide (1)..(1)
  locked nucleic acid nucleotide (2)..(2)
  locked nucleic acid nucleotide (3)..(3)
  locked nucleic acid nucleotide (4)..(4)
  locked nucleic acid nucleotide (5)..(5)
  locked nucleic acid nucleotide (6)..(6)
  locked nucleic acid nucleotide (7)..(7)
  locked nucleic acid nucleotide (8)..(8)
  locked nucleic acid nucleotide (9)..(9)
  locked nucleic acid nucleotide (10)..(10)
  locked nucleic acid nucleotide (11)..(11)
  phosphorylated (11)..(11)
gatctgatcgh

SEQ ID NO: 4 - DNA, 71 bp, Artificial Sequence
  synthesized
  misc_feature (32)..(39): n is a, c, g, or t
aatgatacggcgaccaccgagatctacactannnnnnnnacactctttccctacacgacgctcttccgatc

SEQ ID NO: 5 - DNA, 24 bp, Artificial Sequence
  synthesized
  phosphorothioate bond (22)..(23)
  phosphorothioate bond (23)..(24)
caagcagaagacggcatacgagat

SEQ ID NO: 6 - DNA, 30 bp, hepatitis B virus
acaacattccaccaarctctkctagatccc

SEQ ID NO: 7 - DNA, 39 bp, hepatitis B virus
aagattgacgatatggwtgaggcagtagtcggaacaggg

SEQ ID NO: 8 - DNA, 40 bp, hepatitis B virus
ggtattgtgaggatttttgtcaacaagaaaaaccccgcct

SEQ ID NO: 9 - DNA, 29 bp, hepatitis B virus
gacacacgggtgytccccctagaaaattg

SEQ ID NO: 10 - DNA, 30 bp, hepatitis B virus
acacatccagcgatarccaggacaaytrgg

SEQ ID NO: 11 - DNA, 30 bp, hepatitis B virus
aggtatgttgcccgtttgtcctctamttcc

SEQ ID NO: 12 - DNA, 27 bp, hepatitis B virus
tacaaaacctwcggacggaaaytgcac

SEQ ID NO: 13 - DNA, 30 bp, hepatitis B virus
cccatcccatcatcytgggctttcgcaara

SEQ ID NO: 14 - DNA, 31 bp, hepatitis B virus
aaacagtgggggaaagccctacgaaccactg

SEQ ID NO: 15 - DNA, 31 bp, hepatitis B virus
ggtactgggggccaagtctgtacaacatctt

SEQ ID NO: 16 - DNA, 40 bp, hepatitis B virus
gagtccctttatrccgctrttaccaattttcttttgtctt

SEQ ID NO: 17 - DNA, 35 bp, hepatitis B virus
cccttaacttcatgggatatgtaattggragttgg

SEQ ID NO: 18 - DNA, 40 bp, hepatitis B virus
ttccaatcaataggyctgtttacaggcagtttcckaaaac

SEQ ID NO: 19 - DNA, 45 bp, hepatitis B virus
caatgtggmtatcctgctttratgcctttatatgcatgtatacaa

SEQ ID NO: 20 - DNA, 30 bp, hepatitis B virus
tgtttacacagaaaggccttgtaagttggc

SEQ ID NO: 21 - DNA, 30 bp, hepatitis B virus
gccccaacccgtgggggttgcgtcagcaaa

SEQ ID NO: 22 - DNA, 30 bp, hepatitis B virus
agckgctaggagttccgcagtatggatcgg

SEQ ID NO: 23 - DNA, 30 bp, hepatitis B virus
gttgtcctctctcggaaatacaccgccttt

SEQ ID NO: 24 - DNA, 30 bp, hepatitis B virus
caactggatcctgcgcgggacgtcctttgt

SEQ ID NO: 25 - DNA, 30 bp, hepatitis B virus
ccgaccacggggcgcacctctctttacgcg

SEQ ID NO: 26 - DNA, 30 bp, hepatitis B virus
acgtgcagaggtgaagcgaagtgcacacgg

SEQ ID NO: 27 - DNA, 17 bp, hepatitis B virus
gaccaccgtgaacgccc

SEQ ID NO: 28 - DNA, 21 bp, hepatitis B virus
aggtcttgcccaaggtcttac

SEQ ID NO: 29 - DNA, 22 bp, hepatitis B virus
ttgcacaacaggactcttggac

SEQ ID NO: 30 - DNA, 25 bp, hepatitis B virus
aacgaccgaccttgaggcatacttc

SEQ ID NO: 31 - DNA, 31 bp, hepatitis B virus
ccgaccttgaggcatacttcaaagactgttt

SEQ ID NO: 32 - DNA, 17 bp, hepatitis B virus
gagttrggggaggagat

SEQ ID NO: 33 - DNA, 26 bp, hepatitis B virus
trggggaggagataaggttaaaggtc

SEQ ID NO: 34 - DNA, 35 bp, hepatitis B virus
cctctgcctaatcatctcatgttcatgtcctactg

SEQ ID NO: 35 - DNA, 35 bp, hepatitis B virus
ggggcatggacattgacccstataaagaatttgga

SEQ ID NO: 36 - DNA, 30 bp, hepatitis B virus
accgcctctgctctgtatcgggaggcctta

SEQ ID NO: 37 - DNA, 30 bp, hepatitis B virus
tgttggggtgagttgatgaatctrgccacc

SEQ ID NO: 38 - DNA, 45 bp, hepatitis B virus
atttttaggcccatattaacrttgacatagctgactactaattcc

SEQ ID NO: 39 - DNA, 40 bp, hepatitis B virus
caccaaataytcaagracagtttctcttccaaaagtaagr

SEQ ID NO: 40 - DNA, 36 bp, hepatitis B virus
gtagtttccggaagtgttgataagataggggcattt

SEQ ID NO: 41 - DNA, 30 bp, hepatitis B virus
tccctcgcctcgcagacgaaggtctcaatc

SEQ ID NO: 42 - DNA, 37 bp, hepatitis B virus
tagaagaataaagcccagtaaagtttcccaccttatg

SEQ ID NO: 43 - DNA, 40 bp, hepatitis B virus
tttcctsacattcatctacaggaggacattrttratagat

SEQ ID NO: 44 - DNA, 44 bp, hepatitis B virus
ccgtattatccwgarcatgcagttaatcattacttcaaaactag

SEQ ID NO: 45 - DNA, 30 bp, hepatitis B virus
caaaatgaggcgctrcgtgtagtytctcty

SEQ ID NO: 46 - DNA, 30 bp, hepatitis B virus
ggaggttggtcttccaaacctcgacaaggc

SEQ ID NO: 47 - DNA, 30 bp, hepatitis B virus
ccagttggaccctgcrttcrragccaactc

SEQ ID NO: 48 - DNA, 24 bp, hepatitis B virus
tcatcctcaggccatgcagtggaa

SEQ ID NO: 49 - DNA, 30 bp, hepatitis B virus; identical to SEQ ID NO: 6

SEQ ID NO: 50 - DNA, 39 bp, hepatitis B virus; identical to SEQ ID NO: 7

SEQ ID NO: 51 - DNA, 40 bp, hepatitis B virus; identical to SEQ ID NO: 8

SEQ ID NO: 52 - DNA, 29 bp, hepatitis B virus
gacacacgggtgytccccctagaaaattg

SEQ ID NO: 53 - DNA, 30 bp, hepatitis B virus; identical to SEQ ID NO: 10

SEQ ID NO: 54 - DNA, 30 bp, hepatitis B virus; identical to SEQ ID NO: 11

SEQ ID NO: 55 - DNA, 27 bp, hepatitis B virus
tacaaaacctwcggacggaaaytgcac

SEQ ID NO: 56 - DNA, 30 bp, hepatitis B virus; identical to SEQ ID NO: 13

SEQ ID NO: 57 - DNA, 31 bp, hepatitis B virus; identical to SEQ ID NO: 14

SEQ ID NO: 58 - DNA, 31 bp, hepatitis B virus; identical to SEQ ID NO: 15

SEQ ID NO: 59 - DNA, 40 bp, hepatitis B virus; identical to SEQ ID NO: 16

SEQ ID NO: 60 - DNA, 35 bp, hepatitis B virus; identical to SEQ ID NO: 17

SEQ ID NO: 61 - DNA, 40 bp, hepatitis B virus; identical to SEQ ID NO: 18

SEQ ID NO: 62 - DNA, 45 bp, hepatitis B virus; identical to SEQ ID NO: 19

SEQ ID NO: 63 - DNA, 30 bp, hepatitis B virus; identical to SEQ ID NO: 20

SEQ ID NO: 64 - DNA, 30 bp, hepatitis B virus; identical to SEQ ID NO: 21

SEQ ID NO: 65 - DNA, 30 bp, hepatitis B virus; identical to SEQ ID NO: 22

SEQ ID NO: 66 - DNA, 30 bp, hepatitis B virus; identical to SEQ ID NO: 23

SEQ ID NO: 67 - DNA, 30 bp, hepatitis B virus; identical to SEQ ID NO: 24

SEQ ID NO: 68 - DNA, 30 bp, hepatitis B virus; identical to SEQ ID NO: 25

SEQ ID NO: 69 - DNA, 30 bp, hepatitis B virus; identical to SEQ ID NO: 26

SEQ ID NO: 70 - DNA, 17 bp, hepatitis B virus
gaccaccgtgaacgccc

SEQ ID NO: 71 - DNA, 21 bp, hepatitis B virus
aggtcttgcccaaggtcttac

SEQ ID NO: 72 - DNA, 22 bp, hepatitis B virus
ttgcacaacaggactcttggac

SEQ ID NO: 73 - DNA, 26 bp, hepatitis B virus
aacgacccgaccttgaggcatacttc

SEQ ID NO: 74 - DNA, 26 bp, hepatitis B virus
trggggaggagataaggttaaaggtc

SEQ ID NO: 75 - DNA, 22 bp, hepatitis B virus
ttacataagaggactcttggac

SEQ ID NO: 76 - DNA, 26 bp, hepatitis B virus
trggggaggagattaggttaaaggtc

SEQ ID NO: 77 - DNA, 26 bp, hepatitis B virus
tgggggaggagattaggttaatgatc

SEQ ID NO: 78 - DNA, 35 bp, hepatitis B virus; identical to SEQ ID NO: 34

SEQ ID NO: 79 - DNA, 35 bp, hepatitis B virus; identical to SEQ ID NO: 35

SEQ ID NO: 80 - DNA, 30 bp, hepatitis B virus; identical to SEQ ID NO: 36

SEQ ID NO: 81 - DNA, 30 bp, hepatitis B virus; identical to SEQ ID NO: 37

SEQ ID NO: 82 - DNA, 45 bp, hepatitis B virus; identical to SEQ ID NO: 38

SEQ ID NO: 83 - DNA, 40 bp, hepatitis B virus; identical to SEQ ID NO: 39

SEQ ID NO: 84 - DNA, 36 bp, hepatitis B virus; identical to SEQ ID NO: 40

SEQ ID NO: 85 - DNA, 30 bp, hepatitis B virus; identical to SEQ ID NO: 41

SEQ ID NO: 86 - DNA, 37 bp, hepatitis B virus; identical to SEQ ID NO: 42

SEQ ID NO: 87 - DNA, 40 bp, hepatitis B virus; identical to SEQ ID NO: 43

SEQ ID NO: 88 - DNA, 44 bp, hepatitis B virus; identical to SEQ ID NO: 44

SEQ ID NO: 89 - DNA, 30 bp, hepatitis B virus; identical to SEQ ID NO: 45

SEQ ID NO: 90 - DNA, 30 bp, hepatitis B virus; identical to SEQ ID NO: 46

SEQ ID NO: 91 - DNA, 30 bp, hepatitis B virus; identical to SEQ ID NO: 47

SEQ ID NO: 92 - DNA, 24 bp, hepatitis B virus
tcatcctcaggccatgcagtggaa

SEQ ID NO: 93 - DNA, 45 bp, hepatitis B virus
aactttaggcccatattagtrttgacatagctgactactaggtcy

SEQ ID NO: 94 - DNA, 39 bp, hepatitis B virus
aagattgacgatatgggagaggcagtagtcggaacaggg

SEQ ID NO: 95 - DNA, 39 bp, hepatitis B virus
aagattgacgatatggmagaggcagtattctgarcaggg

SEQ ID NO: 96 - DNA, 39 bp, hepatitis B virus
aagattgacgatawgggagaggcagtagtcrgaacaggg

SEQ ID NO: 97 - DNA, 30 bp, hepatitis B virus
acarccttccaccaarctctkcaagatccc

SEQ ID NO: 98 - DNA, 31 bp, hepatitis B virus
tatttycctgctggtggctccagttcmggaa

SEQ ID NO: 99 - DNA, 41 bp, hepatitis B virus
acatccagcgataaccaggacaagttggaggacargaggtt

SEQ ID NO: 100 - DNA, 41 bp, hepatitis B virus
acatccagcgatarccaggacaarttggaggacaasaggtt

SEQ ID NO: 101 - DNA, 30 bp, hepatitis B virus
accgcctcagctctgtatcgggaggcctta

SEQ ID NO: 102 - DNA, 31 bp, hepatitis B virus
agaggttccttgagcaggaatcgtgcaggtt

SEQ ID NO: 103 - DNA, 31 bp, hepatitis B virus
agcagcaggatgaagaggaakatgataaaac

SEQ ID NO: 104 - DNA, 38 bp, hepatitis B virus
aggagccacaaaggttccacgcatgcgcygatggccya

SEQ ID NO: 105 - DNA, 38 bp, hepatitis B virus
aggagccasaaaggttccacgcatgcgccgatggccya

SEQ ID NO: 106 - DNA, 31 bp, hepatitis B virus
agtgactggagatttgggactgcgaattttg

SEQ ID NO: 107 - DNA, 31 bp, hepatitis B virus
agtgattggaggttggggactgcgaattttg

SEQ ID NO: 108 - DNA, 45 bp, hepatitis B virus
atctttaggcccatattagtrttgacatagttgactactagatcc

SEQ ID NO: 109 - DNA, 41 bp, hepatitis B virus
atgggagtgggcctcagyccgtttctcctggctcagtttac

SEQ ID NO: 110 - DNA, 45 bp, hepatitis B virus
caatgtggmtatcctgcyttratgcctttrtatgcatgtatacaa

SEQ ID NO: 111 - DNA, 45 bp, hepatitis B virus
caatgtggwtatcctgctttratgccyttgtatgcatgtattcaa

SEQ ID NO: 112 - DNA, 29 bp, hepatitis B virus
caggaytcctaggacccctgctcgtgtta

SEQ ID NO: 113 - DNA, 30 bp, hepatitis B virus
ccagttggatccagccttcagagcaaacac

SEQ ID NO: 114 - DNA, 30 bp, hepatitis B virus
cccatcccatcatcytgggctttcggaaaa

SEQ ID NO: 115 - DNA, 35 bp, hepatitis B virus
ccctaaayttcatgggytatgtaattggragttgg

SEQ ID NO: 116 - DNA, 31 bp, hepatitis B virus
ccgcaagatcayatyrtacaaaaaatcaagg

SEQ ID NO: 117 - DNA, 31 bp, hepatitis B virus
ccrcargaacatattgtacaaaaaatcaarc

SEQ ID NO: 118 - DNA, 35 bp, hepatitis B virus
cctctgcctaatcatctcwtgttcatgtcctactg

SEQ ID NO: 119 - DNA, 44 bp, hepatitis B virus
ccttattatccagagcatgtagttaatcattacttccagacrag

SEQ ID NO: 120 - DNA, 44 bp, hepatitis B virus
ccttattatccwgarcatstagttaatcattacttccaaacyag

SEQ ID NO: 121 - DNA, 36 bp, hepatitis B virus
cgcagccggtctggagcgaaactcatcggaactgac

SEQ ID NO: 122 - DNA, 36 bp, hepatitis B virus
cgcagccggtctggagcgaaacttatcggaaccgac

SEQ ID NO: 123 - DNA, 36 bp, hepatitis B virus
cgcagcmggtctggagcgaaaattatcggaactgay

SEQ ID NO: 124 - DNA, 36 bp, hepatitis B virus
ctaaacctttaccccgttgcccggcaacggtcaggt

SEQ ID NO: 125 - DNA, 36 bp, hepatitis B virus
ctaaacctttaccccgttgctcggcaacggccaggt

SEQ ID NO: 126 - DNA, 36 bp, hepatitis B virus
ctaaacctttaccccgttgctcggcaacggtcaggt

SEQ ID NO: 127 - DNA, 34 bp, hepatitis B virus
ctctgcctaatcatctcttgtacatgtcctactk

SEQ ID NO: 128 - DNA, 31 bp, hepatitis B virus
ctgggtgggwartaatttggaagayccagcr

SEQ ID NO: 129 - DNA, 35 bp, hepatitis B virus
ctytaaatttcatgggytatgtcattggragttat

SEQ ID NO: 130 - DNA, 29 bp, hepatitis B virus
gacacrcggkwgytccccctagaaaattg

SEQ ID NO: 131 - DNA, 31 bp, hepatitis B virus
gaggactggggaccctgcrccgaacatggag

SEQ ID NO: 132 - DNA, 31 bp, hepatitis B virus
gaggattggggaccctgcgctgaacatggag

SEQ ID NO: 133 - DNA, 40 bp, hepatitis B virus
gagtccctttwtrccgctrttaccaattttcttttgtctt

SEQ ID NO: 134 - DNA, 30 bp, hepatitis B virus
gccccarccagtgggggttgcgtcagcaaa

SEQ ID NO: 135 - DNA, 31 bp, hepatitis B virus
gccgcgtcgcagaagatctcaatctcgggaa

SEQ ID NO: 136 - DNA, 36 bp, hepatitis B virus
gctgtaggcataaattggtctgcgcaccagcaccat

SEQ ID NO: 137 - DNA, 36 bp, hepatitis B virus
gctgtaggcataaattggtctgttcaccagcaccat

SEQ ID NO: 138 - DNA, 36 bp, hepatitis B virus
ggggcagcaaagcccaaaagacccacaattckttga

SEQ ID NO: 139 - DNA, 35 bp, hepatitis B virus
ggggcatggacattgacccktataaagaatttgga

SEQ ID NO: 140 - DNA, 31 bp, hepatitis B virus
ggtattgggggccaagtctgtacarcatctt

SEQ ID NO: 141 - DNA, 40 bp, hepatitis B virus
ggtattgtgaggattyttgtcaacaagaaaaaccccgcct

SEQ ID NO: 142 - DNA, 30 bp, hepatitis B virus
gtygtcctctcccgsaaatatacagcgttt

SEQ ID NO: 143 - DNA, 27 bp, hepatitis B virus
taccaaaccttcggacggaaaytgcac

SEQ ID NO: 144 - DNA, 37 bp, hepatitis B virus
tagaagaataaagcccmgtaaagtttcccaccttatg

SEQ ID NO: 145 - DNA, 22 bp, hepatitis B virus
tcatcctcaggccatgcagtgg

SEQ ID NO: 146 - DNA, 30 bp, hepatitis B virus
tgctggggggarttgatgactctrgctacc

SEQ ID NO: 147 - DNA, 30 bp, hepatitis B virus
tgtttacayagaaaggccttgtaagttggc

SEQ ID NO: 148 - DNA, 40 bp, hepatitis B virus
ttccaatcaataggtctatttacaggaagttttckaaaac

SEQ ID NO: 149 - DNA, 40 bp, hepatitis B virus
ttccaatcaataggyctgttaacaggaagttttckaaaac

SEQ ID NO: 150 - DNA, 36 bp, hepatitis B virus
ttgggtatacatttgaaccctaacaaaaccaaacga

SEQ ID NO: 151 - DNA, 37 bp, hepatitis B virus
ttgggtatacatttgaaccctaataaaacaaaaacgt

SEQ ID NO: 152 - DNA, 36 bp, hepatitis B virus
ttgggtatacatttgaaccctaataaaaccaaacgt

SEQ ID NO: 153 - DNA, 22 bp, hepatitis B virus
ttrcacaagaggactcttggac

SEQ ID NO: 154 - DNA, 40 bp, hepatitis B virus
tttcctaarattcatttacawgaggacattrttaatagat

SEQ ID NO: 155 - DNA, 40 bp, hepatitis B virus
tttcctaatatacatttacagcaggacattatcaaaaaat

SEQ ID NO: 156 - DNA, 31 bp, hepatitis B virus
ctctatcgtccccttcttcatctgccgttcc

SEQ ID NO: 157 - DNA, 31 bp, hepatitis B virus
ctctaccgyccscttcttcatctgccgtwcc

SEQ ID NO: 158 - DNA, 35 bp, hepatitis B virus
ggaaagaagtcagaaggcaaaaacgagagtaactc

SEQ ID NO: 159 - DNA, 35 bp, hepatitis B virus
ggaaagaagtcagaaggcaaaaaagagagtaactc

SEQ ID NO: 160 - DNA, 31 bp, hepatitis B virus
tctcctgarcattgytcacctcaccatacrg

SEQ ID NO: 161 - DNA, 31 bp, hepatitis B virus
tctcctgagcattgttcacctcaccatactg

SEQ ID NO: 162 - DNA, 31 bp, hepatitis B virus
tctccggaacattgttcacctcaccatacag

SEQ ID NO: 163 - DNA, 26 bp, hepatitis B virus
tatctttaatcctgaatggcaaactc

SEQ ID NO: 164 - DNA, 26 bp, hepatitis B virus
tgtctttaatcctcattggaaaacac

SEQ ID NO: 165 - DNA, 26 bp, hepatitis B virus
tgtctttaatcctgartggcaaactc

SEQ ID NO: 166 - DNA, 31 bp, hepatitis B virus
aacctagcaggcataatcaattkcartcttc

SEQ ID NO: 167 - DNA, 31 bp, hepatitis B virus
aacctagcaggcataattaattttagtctcc

SEQ ID NO: 168 - DNA, 31 bp, hepatitis B virus
aacctagcaggcataattaattttaatctcc

SEQ ID NO: 169 - DNA, 29 bp, hepatitis B virus
catgctgtagctcttgttcccaagaatat

SEQ ID NO: 170 - DNA, 41 bp, hepatitis B virus
aatctttctgtycccaatcctctgggattctttcccgatca

SEQ ID NO: 171 - DNA, 41 bp, hepatitis B virus
aatctttccaccagcaatcctctgggattctttcccgacca

SEQ ID NO: 172 - DNA, 30 bp, hepatitis B virus
ccaacaaggtaggagykggagcattcgggc

SEQ ID NO: 173 - DNA, 41 bp, hepatitis B virus
atatgccctgagcctgagggctccaccccaaaacacctccg

SEQ ID NO: 174 - DNA, 41 bp, hepatitis B virus
gtatgccctgagcctgagggctccaccccaaaagkccyccr

SEQ ID NO: 175 - DNA, 41 bp, hepatitis B virus
atatgccctgagcctgagggctccaccccaaaagaccgccg

SEQ ID NO: 176 - DNA, 29 bp, Artificial Sequence
  synthesized
  phosphorphylated (29)..(29)
gtgtagatctcggtggtcgccgtatcatt

SEQ ID NO: 177 - DNA, 24 bp, Artificial Sequence
  synthesized
  locked nucleic acid nucleotide (10)..(10)
  locked nucleic acid nucleotide (22)..(22)
  phosphorylated (24)..(24)
caagcagaagacggcatacgagat

SEQ ID NO: 178 - DNA, 120 bp, homo sapiens
cgaccttgaggcatacttcaaagactgtttgtttaaagactgggaggagttgggggaggagattaggaggctgtaggcataaaggaaggggaggggctgggagggcccggagggggctgg

SEQ ID NO: 179 - DNA, 109 bp, homo sapiens
gggggaggagataaggttaaaggtctttgtactaggaggctgtaggcataaattggtctgcccagccccctccgggccctcccagcccctccccttcctttccgcggcc

SEQ ID NO: 180 - DNA, 151 bp, homo sapiens
gaggagattaggctaaaggtctttgtactaggaggctgtaggcataaattggtctgttcaccagcaccatgcaacggagctcataacctgatcagctttctcttcttctctctgtttttgtcttgtttggtgtgtttccttggggtcatgg

SEQ ID NO: 181 - DNA, 131 bp, homo sapiens
gggggaggagataaggttaaaggtctttgtactaggaggctgtaggcataaattggtctgttcaccagcaccatgcaactttttccttttctatatcaattgttgatactccaataatattaattgctaag

SEQ ID NO: 182 - DNA, 85 bp, homo sapiens
gggggaggagataaggttaaaggtctttgtactaggaggctgtaggcataaattttatcttcatataaaatctagacggaagcat

SEQ ID NO: 183 - DNA, 149 bp, homo sapiens
actaggaggctgtaggcataaattggtctgttcaccagcaccatgcaactttttcttatgaatgttttctatatttcaaagccctgctcaaacaccacctcctccagaaaggctcctggtatcctctttcttttctaacctagaaaaga

SEQ ID NO: 184 - DNA, 146 bp, homo sapiens
gcctaatcatgtcatgttcatgtcctactgttcaagcctccaagctgtgccttgggtggctttggggcatgcgggtgcccgggtcgcgggtgacaggccaccccgccatcggccatcttcctggctcgcccggccgcccgcgcgca

SEQ ID NO: 185 - DNA, 151 bp, homo sapiens
gactctcagcaatgtcaacgaccgaccttgaggcatacttcaaagactgtttgtttaaggactgggaggagttgggggaggagattaggttaaagattaccatgttgcccaggctggtcttgaacacctggcctcaagggacgctcccagc

SEQ ID NO: 186 - DNA, 151 bp, homo sapiens
cacgtcgcatggagaccaccgtgaacgcccaccaagtcttgcccaaggtcttacataagcggactcttggactcccagcaatgtcaacgaccgaccttgaggcgtacttcaaaacccagacccagctcaggcatcaccacctccaggcagc

SEQ ID NO: 187 - DNA, 151 bp, homo sapiens
tggactttcagcaatgtcaatgaccgaccttgaggcatacttcaaagactgtgtgtttactgagtgggaggagttgggggagggactagctcattaatcattgtgtcaaacctggcaccgtgcctgaaacacagtagcctctcaataaata

SEQ ID NO: 188 - DNA, 109 bp, homo sapiens
ttgcacaacaggactcttggacttacaccagtggtttgccggggaatcttgagcctttggccacagactgaaggctgcactgtcagcttccctacttttgaggctttcg

SEQ ID NO: 189 - DNA, 151 bp, homo sapiens
actaggaggctgtaggcataaattggtctgttcaccagcaccatgcaacttttttccgaacctgtgtactaaactgcctgggggcagctctcatcactgctgtagaacaaagtcccacatagagccaatggccaagaaccagttaataaaa

SEQ ID NO: 190 - DNA, 131 bp, homo sapiens
gagtgggaggagttgggggaggagattaggttaaaggtctttgtactaggaggctgcatggccggaagtcttacatgtcttgggagtttgtggggagggggtgaaatcgggacttcttctagctgccacgg

SEQ ID NO: 191 - DNA, 115 bp, homo sapiens
gggggaggagataaggttaaaggtctttgtactagtaggctgtaggcataaattgcctacagcaatgtatagattttaaataaatgcttgctgacttactatgacctactggtag

SEQ ID NO: 192 - DNA, 151 bp, homo sapiens
gactcttggactcccagcaatgtcaacgaccgaccttgaggcctacttcaaagactgtgtgtttaaggactgggaggagctgggggaggagattaggttaatgatctttgtactaggaggaacatgcccaagaaattggcgacataccagc

SEQ ID NO: 193 - DNA, 147 bp, homo sapiens
tcttgcataagaggactcttggactttcggcaatgtcaacgaccgaccttgaggcatacttcaaagactgtgtgtttaactcatctgtccaaacccaaagaatggactcagagacccagagaacaacgaaagtgacggtttgttctt

SEQ ID NO: 194 - DNA, 156 bp, homo sapiens
ttgcacaacaggactcttggactctcagcaatgtcaacgaccgaccttgaggcatacttcaaagactgtttgtttaaagactgggaggagttgcatctaactcaggttttcaactagtcttaccattgaaagaactattgtggcaaagacggaatg

SEQ ID NO: 195 - DNA, 150 bp, homo sapiens
aaagactgggaggagttgggggaggagattaggttaaaggtctttgtactaggaggctgtaggcataaattggtctgtttgaagttgtccagaaactgacctttgaatatccggatgcacgagattccctgaaaggggaacaataaatgt

SEQ ID NO: 196 - DNA, 152 bp, homo sapiens
caaggtcttacataagcggactcttggactctcagcaatgtcaacgaccgaccttgaggcgtacttcaaaggtgttacaggtagttagacaggcatgagcagggcaggagagaacgctcccctgactcaccaggaatgtcaggcaatcattg

SEQ ID NO: 197 - DNA, 144 bp, homo sapiens
tgggaggagttgggggaggagattaggttaatgatctttgtactaggaggctgtaggcataaattggtgtgttcacctgcaccatgcaactttttctggggatggggatgtggcagttgtggactgaagttgtactgagtggtg

SEQ ID NO: 198 - DNA, 150 bp, homo sapiens
ttaggttaatgatctttgtactaggaggctgtaggcataaattggtctgacccgcccttctctgcccagcacttttctgcccccctccctctggaacacagagtggcagtttccacaagcactaagcatcctcttcccaaaagacccagc

SEQ ID NO: 199 - DNA, 124 bp, homo sapiens
aacagtctttgaagtacgcctcaaggtcggtcgttgacattgctgagagtccaagagtccgcttatgtaagaccttgggcaagacctggtgggcgtttggtggcattgcaagtgtactgtttaa

SEQ ID NO: 200 - DNA, 174 bp, homo sapiens
cacaacaggactcttggactctcagcaatgtcaacgaccgaccttgaggcatacttcaaagactgtgtgtttaaagactgggaggagttgggggaggagattaggttaaaggtctttgtactaggaggctgtaggcataaattggtctggacctgcatcatccggactccatac

SEQ ID NO: 201 - DNA, 210 bp, homo sapiens
tgcacaacaggactcttggactctcagcaatgtcaacgaccgaccttgaggcatacttcaaagactgtgtgtttaaagactgggaggagttgggggaggagattaggttaaaggtctttgtactaggaggctgtaggcataaattggtctgcccgcggcccgggcactcaccgctccctgcgctccctcggcatgatggggctgctccgg

SEQ ID NO: 202 - DNA, 150 bp, homo sapiens
tgcacaacaggactcttggactctcagcaatgtcaacgaccgaccttgaggcatacttcaaagactgtgtgtttaaagactgggaggagttgggggaggagattaggttaaaggtgtctggtattatttctgggttctctattctgttcc

SEQ ID NO: 203 - DNA, 105 bp, homo sapiens
tttaaagactgtgaggagttgggggaggagattaggttaacggtctttgtgctgtggagggaagactaagtagagacgcggatgtttatggcagtgaaactgttc

SEQ ID NO: 204 - DNA, 112 bp, homo sapiens
cgaccttgaggcatacttcaaagactgtttcaagaaactgagtgagtaggctctggaaattggaagtgatcttagtatttaagttcagtcactcaactacaatctctgaaac

SEQ ID NO: 205 - DNA, 129 bp, homo sapiens
cgaccttgaggcatacttcaaagactgttttaccagacactcacatggcttcctcgctgtcttcctgtggtggcacacgcctgtagtcccagctacttgggaggctgaggcaggagaattgcttgaacc

SEQ ID NO: 206 - DNA, 141 bp, homo sapiens
gggggaggagataaggttaaaggtctttgtactaggaggctgtaggcataaattggtctgttcaccagcaccatgcaactttttctggtgaaaagctaaacacaggagatatttttaagcttcactcatacagaaaataca

SEQ ID NO: 207 - DNA, 151 bp, homo sapiens
gggggaggagataaggttaaaggtcttgtttgtactaggaggctgtaggcataaattggcaggacccaggggagcagccagcactgcgcatgctgggagtgttcaataaatacaggctgaatgaatgaatgaactgatgcatccaaaactt

SEQ ID NO: 208 - DNA, 123 bp, homo sapiens
cgaccttgaggcatacttcaaagactgtttggaaaaaatgtaaacatatcagccctgagcaagacagccaaaccaaaacaaccacagcgagggattctgattcctttgacagactctgtttct

SEQ ID NO: 209 - DNA, 78 bp, homo sapiens
cgaccttgaggcatacttcaaagactgtttgccttttcccctaatcccctttccccactggtacagggtggagaggtc

SEQ ID NO: 210 - DNA, 151 bp, homo sapiens
gactcccagcaatgtcaacgaccgaccttgaggcctacttcaaagactgtgtgtttaaggactgggaggagctgggggaggagattaggttaaaggtctttgtattaggaggctgtaggcataaattggtctgcgtcaccctccagaagga

SEQ ID NO: 211 - DNA, 172 bp, homo sapiens
cacaacaggactcttggactcccagcaatgtcaacgaccgaccttgaggcctacttcaaagactgtgtgtttaaggacttggaggagctgggggaggagattaggttaaaggtctttgtattaggatgctgtaggcataaattggtctgccttgactaaagcccatgggcca

SEQ ID NO: 212 - DNA, 18 bp, artificial
  synthesized
cagcgctgcctgaaactc

SEQ ID NO: 213 - DNA, 18 bp, artificial
  synthesized
gtcctgccccttcacctt